(19) United States
(12) Patent Application Publication
(10) Pub. No.: US 2022/0227815 A1
(43) Pub. Date: Jul. 21, 2022

(54) COMPOSITIONS AND METHODS USEFUL IN TREATING BRAIN DISEASES

(71) Applicant: University of Kansas, Lawrence, KS (US)

(72) Inventors: Teruna J. Siahaan, Lawrence, KS (US); Brian Matthew Kopec, Lawrence, KS (US); Kavisha Raneendri Ulapane, Battaramulla (LK)

(21) Appl. No.: 17/615,032

(22) PCT Filed: Jun. 21, 2020
(86) PCT No.: PCT/US2020/038865

§ 371 Date: Nov. 29, 2021

Related U.S. Application Data
(60) Provisional application No. 62/865,105, filed on Jun. 21, 2019.

(51) Int. Cl. C07K 7/64; C07K 7/06; A61K 38/12; A61K 45/06

(52) U.S. Cl. CPC C07K 7/64; A61K 45/06; A61K 38/12; C07K 7/06

(57) ABSTRACT

Compounds, compositions, and methods are provided that are useful in treating brain diseases by effecting delivery across the blood brain barrier of molecules that otherwise do not (or insignificantly) pass across the blood brain barrier, where compounds of the present technology include but are not limited to cyclo(1,6)SHAVSS (“HAVN1”), cyclo(1,5)SHAVS (“HAVN2”), cyclo(1, 8)TPP V SHAV (“cyclic ADTHAV”), cyclo(1,6)ADTPPV (“ADTN1”), cyclo(1,5)DTPPV (“ADTN2”), acetyl-TPPVSHAV-NH2 (“linear ADTHAV”), and pharmaceutically acceptable salts thereof.

### CROSS-REFERENCE TO RELATED APPLICATIONS

This application claims the benefit of and priority to U.S. Provisional Appl. No. 62/865,105, filed Jun. 21, 2019, which in incorporated herein by reference in its entirety.

### U.S. GOVERNMENT RIGHTS

This invention was made with government support under AG035982 and NS075374 awarded by the National Institutes of Health. The government has certain rights in the invention.

### FIELD

The present technology is directed to compounds, compositions, and methods useful in treating brain diseases by effecting delivery across the blood brain barrier of molecules that otherwise do not (or insignificantly) pass across the blood brain barrier.

### SUMMARY

In an aspect, the present technology provides a compound that is cyclo(1,6)SHAVSS (SEQ ID NO: 1; “HAVN1”) or a pharmaceutically acceptable salt thereof, cyclo(1,5)SHAVS (SEQ ID NO: 2; “HAVN2”) or a pharmaceutically acceptable salt thereof, cyclo(1,8)TPPVSHAV (SEQ ID NO: 3; “cyclic-ADTHAV”) or a pharmaceutically acceptable salt thereof, cyclo(1,6)ADTPPV (SEQ ID NO: 4; “ADTN1”) or a pharmaceutically acceptable salt thereof, cyclo(1,5)DTPPV (SEQ ID NO: 5; “ADTN2”) or a pharmaceutically acceptable salt thereof, or acetyl-TPPVSHAV-NH₂ (SEQ ID NO: 6; “linear ADTHAV”) or a pharmaceutically acceptable salt thereof.

In a related aspect of the present technology, a composition is provided that includes a pharmaceutically acceptable carrier and one or more of HAVN1, HAVN2, cyclic-ADTHAV, ADTN1, ADTN2, linear ADTHAV, and a pharmaceutically acceptable salt of any one or more thereof. In a related aspect, pharmaceutical compositions and medicaments are provided that include an effective amount of one or more of HAVN1, HAVN2, cyclic-ADTHAV, ADTN1, ADTN2, linear ADTHAV, and a pharmaceutically acceptable salt of any one or more thereof as well as include a pharmaceutically acceptable carrier, wherein the effective amount is effective for one or more of treating a brain disease, imaging a brain disease, and diagnosing a brain disease. In a further related aspect, a method is provided that includes administering one or more of HAVN1, HAVN2, cyclic-ADTHAV, ADTN1, ADTN2, linear ADTHAV, and a pharmaceutically acceptable salt of any one or more thereof to a subject suffering from a brain disease. In a further related aspect, a method is provided that includes administering a pharmaceutical composition or medicament to a subject suffering from a brain disease, where the pharmaceutical composition or medicament includes an effective amount of one or more of HAVN1, HAVN2, cyclic-ADTHAV, ADTN1, ADTN2, linear ADTHAV, and a pharmaceutically acceptable salt of any one or more thereof as well as include a pharmaceutically acceptable carrier, wherein the effective amount is effective for one or more of treating a brain disease, imaging a brain disease, and diagnosing a brain disease.

In an aspect, a pharmaceutical composition is provided that includes a pharmaceutically acceptable carrier and an effective amount of one or more of acetyl-SHAVSS-NH₂ (SEQ ID NO: 7; “HAV6”) or a pharmaceutically acceptable salt thereof, cyclo(1,7)acetyl-CDTPPVC-NH₂ (SEQ ID NO: 8; “ADTC5”) or a pharmaceutically acceptable salt thereof, acetyl-SHAVAS-NH₂ (SEQ ID NO: 9; “HAV4”) or a pharmaceutically acceptable salt thereof, and cyclo(1,6)acetyl-CSHAVC-NH₂ (SEQ ID NO: 10; “cHAVc3”) or a pharmaceutically acceptable salt thereof, wherein the effective amount is effective for one or more of treating a brain disease, imaging a brain disease, and diagnosing a brain disease. In a related aspect, a method is provided that includes administering to a subject suffering from a brain disease one or more of HAV6, ADTC5, HAV4, cHAVc3, and a pharmaceutically acceptable salt of any one or more thereof. In a further related aspect, a method is provided that includes administering to a subject suffering from a brain disease a pharmaceutical composition where the pharmaceutical composition includes an effective amount of one or more of HAV6, ADTC5, HAV4, cHAVc3, and a pharmaceutically acceptable salt of any one or more thereof as well as include a pharmaceutically acceptable carrier, wherein the effective amount is effective for one or more of treating a brain disease, imaging a brain disease, and diagnosing a brain disease.

### BRIEF DESCRIPTION OF THE DRAWINGS

FIG. 1 provides quantitative levels of brain deposition of IRdye800CW-IgG mAb using NIRF imaging in pmol/g brain after delivery of IgG mAb alone (21.6 nmol/kg) or delivered with HAV6, HAVN1, or HAVN2 (13 μmol/kg) in C57BL/6 mice. The asterisk (*) designates a significant difference in HAVN1- or HAVN2-treated groups compared to control with p<0.05. Error bars show the mean±SEM with the number of animals, n=3, for each group.

FIG. 2 provides quantitative levels of brain deposition of IRdye800CW-IgG mAb brain deposition using NIRF imaging in pmol/g brain after its administration (21.6 nmol/kg) without a peptide of the present technology as a control group or in the presence of ADTC5, linear ADTHAV, or cyclic ADTHAV (13 μmol/kg) in C57BL/6 mice. The asterisk (*) indicates a significant difference in cyclic ADTC5-, linear ADTHAV-, or cyclic ADTHAV-treated groups compared to control with p<0.05. Error bars show the mean±SEM with the number of animals, n=3, for each group.

FIG. 3 provide results illustrating the effects of linear HAVE, cyclic HAVN1, and cyclic HAVN2 peptides on the peripheral organ deposition of the IRdye800CW-IgG mAb in heart, lung, kidney, spleen, and liver determined using NIRF signal intensity quantitatively in absorption units (A.U.). The IgG mAb deposition was measured by the total NIRF image intensity in each organ. There is no significant difference in the IgG mAb signal intensities for each organ when comparing the control group and peptide-treated group with p>0.05. Error bars show the mean±SEM with the number of animals, n=3, for each group.

FIG. 4 provide results illustrating the effects of cyclic ADTC5, linear ADTHAV, and cyclic ADTHAV peptides on the peripheral organ deposition of the IRdye800CW-IgG mAb in heart, lung, kidney, spleen, and liver determined using NIRF signal intensity quantitatively in absorption units (A.U.). The IgG mAb deposition was measured by the total NIRF image intensity in each organ. There are significance differences in the IgG mAb signal intensities for kidney and heart of ADTC5- or linear ADTHAV-treated mice compared to control (*p<0.05). There are significant differences in the IgG mAb signal in lung, kidney, spleen, and liver from the cyclic ADTHAV-group compared to the control group (*p<0.05). Error bars show the mean±SEM with the number of animals, n=3, for each group.

FIGS. 5A-5B provide results illustrating the effect of treatment of SJL/elite EAE mice, an animal model for MS, with BDNF (5.71 nmol/kg)+ADTC5 (10 μmol/kg; n=7), BDNF alone (5.71 nmol/kg; n=6), ADTC5 alone (10 μmol/kg; n=5), or vehicle (n=5) during remission on days 21, 25, 29, 33, 37, 4; 1, 45, and 48. FIG. 5A provides clinical disease score vs. time of mice treated 8 times with either BDNF+ADTC5, BDNF alone, ADTC5 alone or vehicle; arrows indicate treatment days; FIG. 5B provides a comparison of area under the curve (AUC) of the disease scores from days 21-55 from EAE mice treated with BDNF+ADTC5, BDNF alone, ADTC5 alone, or vehicle. *p≤0.05; one-way ANOVA (95% confidence).

FIGS. 6A-6B provide results illustrating the effects of BDNF (5.71 nmol/kg)+ADTC5 (10 μmol/kg), BDNF alone (5.71 nmol/kg), or vehicle treatments on remyelination in the lateral corpus callosum and surrounding cortex of the brains of SJL/elite EAE mice as stained by Luxol fast blue. FIG. 6A provides a greyscale, binary conversion, and color photomicrograph of myelin images taken under identical exposure of the lateral corpus callosum of EAE mice treated with BDNF+ADTC5, BDNF Alone, or vehicle; red arrows indicate breakages in the myelin; FIG. 6B provides a quantitative myelin densiometric comparison in the brain of BDNF+ADTC5, BDNF Alone, and vehicle treated EAE mice; Scale bar=50 μm; **p≤0.01 ***p≤0.001; one-way ANOVA (95% confidence; n=5).

FIGS. 7A-7B provide results illustrating the effects of BDNF (5.71 nmol/kg)+ADTC5 (10 μmol/kg), BDNF Alone (5.71 nmol/kg), or vehicle treatments on presence of NG2 receptor in the medial corpus callosum of brains of SJL/elite EAE mice as stained by DAB. FIG. 7A provides a color photomicrograph of anti-NG2 staining (brown) taken under identical conditions from the medial corpus callosum for mice treated with BDNF+ADTC5, BDNF alone, vehicle; red arrows point to dense regions of activated NG2-glia; FIG. 7B provides a quantitative NG2 density comparison amongst the EAE mice treated with BDNF+ADTC5, BDNF alone, and vehicle; Scale bar=50 μm; **p≤0.01; one-way ANOVA (95% confidence; n=5).

FIGS. 8A-8D provide results illustrating the effects of BDNF (5.71 nmol/kg)+ADTC5 (10 μmol/kg), BDNF alone (5.71 nmol/kg), or vehicle treatments on mRNA expression of EGR1 and ARC in the cortex of the brains of SJL/elite EAE mice. FIGS. 8A-8B provides a photomicrograph of DAPI (blue), EGR1 (green), ARC (magenta), and composite images taken of the cortex of the midbrain (FIG. 8A) and hindbrain (FIG. 8B) of EAE mice treated with BDNF+ADTC5, BDNF alone, or vehicle. FIG. 8C provides a quantitative comparison of EGR, ARC, and NOS1 mRNA transcript expression, as determined by cell count, for mice treated with BDNF+ADTC5, BDNF alone, or vehicle. FIG. 8D provides a quantitative comparison of DAPI cell count; Scale bar=50 μm; ***p≤0.001; one-way ANOVA (99% confidence; n=5). Contrast and brightness of images were adjusted only for display purposes.

FIGS. 9A-9G provides the results of Western blot detection of recombinant BDNF and pTrkB from mice treated with either BDNF+ADTC5 or BDNF alone. FIG. 9A provides a Western blot probing for recombinant BDNF in the brains of mice that received BDNF (5.71 nmol/kg)+ADTC5 (10 μmol/kg; A1, A2, A3) or BDNF alone (5.71 nmol/kg, B1, B2, B3); represents molecular weight ladder; ‘+’ represents the positive control of recombinant BDNF; red arrows highlight increased recombinant BDNF detection. FIG. 9B provides a Western blot probing for recombinant BDNF after dosage increase in healthy mice that received BDNF (57.1 nmol/kg)+ADTC5 (10 μmol/kg; A1, A2), BDNF (28.6 nmol/kg)+ADTC5 (10 μmol/kg; A3), or), BDNF alone (28.6 nmol/kg; B1, B2, B3); red arrows highlight increased recombinant BDNF detection. FIG. 9C provides a Western Blot probing for pTrkB after dosage increase of healthy mice that received BDNF (57.1 nmol/kg)+ADTC5 (10 μmol/kg; A1, A2), BDNF (28.6 nmol/kg)+ADTC5 (10 μmol/kg; A3), or BDNF alone (28.6 nmol/kg; B1, B2, B3); red arrows highlight increased pTrkB detection. FIG. 9D provides a total protein stain (loading control) for samples treated with BDNF 57.1 nmol/kg or 28.6 nmol/kg in B and C. FIG. 9E provides a graphical representation of recombinant BDNF detection level in mice that received BDNF (57.1 nmol/kg)+ADTC5 (10 μmol/kg; A1, A2), BDNF (28.6 nmol/kg)+ADTC5 (10 μmol/kg; A3), or BDNF alone (28.6 nmol/kg; B1, B2, B3). FIG. 9F provides a graphical representation of pTrkB detection level for mice that received BDNF (57.1 nmol/kg)+ADTC5 (10 μmol/kg; A1, A2), BDNF (28.6 nmol/kg)+ADTC5 (10 μmol/kg; A3), or BDNF alone (28.6 nmol/kg; B1, B2, B3). FIG. 9G provides a graphical representation of total protein loaded among all groups. Contrast and brightness of images were adjusted only for display purposes.

FIGS. 10A-10B provide the results of a Y-maze cognitive assessment of transgenic APP/PS1 mice, an AD animal model after eight injections of BDNF (5.71 nmol/kg)+ADTC5 (10 μmol/kg), BDNF alone (5.71 nmol/kg), or vehicle. FIG. 10A provides the percent of total time spent in the novel arm or third arm of the Y-maze; FIG. 10B provides the total number of entries made into the third arm of the Y-maze. *p<0.05; one-way ANOVA (95% confidence, n=5).

FIGS. 11A-11B provide the results of a novel object recognition (NOR) cognitive assessment of transgenic APP/PS mice after eight injections with BDNF (5.71 nmol/kg)+ADTC5 (10 μmol/kg), BDNF alone (5.71 nmol/kg), or vehicle alone. FIG. 11A provides the percent of total time spent interacting with the novel object; FIG. 11B provides the total amount of time mice spent interaction with either object. *p<0.05; one-way ANOVA (95% confidence, n=5); NS=No Significant.

FIG. 12 provides results illustrating the effect of eight injections of BDNF (5.71 nmol/kg)+ADTC5 (10 μmol/kg), BDNF alone (5.71 nmol/kg), or vehicle in APP/PS1 mice on amyloid plaque loads at the hippocampal region as determined using Congo red staining. Notably, there is no significant difference (NS) in all three groups.

FIGS. 13A-13B provide results illustrating the effect of multiple treatments of APP/PS1 mice with BDNF (5.71 nmol/kg)+ADTC5 (10 μmol/kg), BDNF alone (5.71 nmol/kg), or vehicle on the expression of NG2 receptors in the cortex as stained by DAB. FIG. 13A provides a color photomicrograph of anti-NG2 staining (brown) taken under identical conditions from the cortex of mice treated with BDNF+ADTC5, BDNF alone, and vehicle; red arrows point to dense regions of activated NG2-glia; FIG. 13B provides a quantitative NG2 density comparison among the APP/PS1 mice treated with BDNF+ADTC5, BDNF alone, and vehicle; Scale bar=100 μm; **p≤0.01; NS=No Significant Difference; one-way ANOVA (95% confidence; n=5).

FIGS. 14A-14B provide results illustrating effects of BDNF (5.71 nmol/kg)+ADTC5 (10 μmol/kg), BDNF alone (5.71 nmol/kg), or vehicle treatments on mRNA expression of MAPK1, EGR1, and ARC in the CA1 region of the brain hippocampus from treated APP/PS1 mice. FIG. 14A provides a photomicrograph of DAPI (grey), EGR1 (green), ARC (red), MAPK (cyan) and composite images taken of the hippocampus of APP/PS1 mice treated with BDNF+ADTC5, BDNF alone, or vehicle; FIG. 14B provides a quantitative comparison using fluorescence intensities of MAPK1 EGR1, and ARC, and mRNA transcript expressions after multiple treatments with BDNF+ADTC5, BDNF alone, or vehicle. Scale bar=100 μm; p*≤0.05; ** and ***p≤0.001; one-way ANOVA (99% confidence; n=4); NS=No significant difference. Contrast and brightness of images were adjusted only for display purposes.

FIGS. 15A-15B provide results illustrating the effect of ADTC5 (13 μmol/kg) on improving the brain delivery of IRdye800CW-IgG mAb (26.8 nmol/kg) in SJUelite mice. FIG. 15A provides an image showing whole brain fluorescence of mice that received IRDye800cw-IgG mAb alone (left; n=4) and IRDye800cw-IgG mAb+ADTC5 (right; n=5). FIG. 15B provides the mean fluorescence intensity of IRDye800cw-IgG mAb for quantitative comparison of NIRF signals between mice that received IRDye800cw-IgG mAb+ADTC5 vs. IRDye800cw-IgG mAb alone. Asterisk (*) was used to designate a significant difference between the ADTC5 group and the control group when p<0.05. Error bars show the mean±SE for both groups.

FIGS. 16A-16B provide quantitative comparisons of IRdye800CW-lysozyme (54 nmol/kg) depositions in the brain and other organs when administered alone and along with HAV6 and ADTC5 peptides (13 μmol/kg). FIG. 16A provides quantitative comparisons of lysozyme brain depositions in pmol/g brain for control, HAV6-, and ADTC5-treated mice. FIG. 16B Comparisons of lysozyme depositions in various organs using tissue NIRF signal intensities. A significant difference between peptide and control groups with p<0.05 was designated using an asterisk (*) symbol. The mean±SE was used in the error bars for all groups.

FIGS. 17A-17B provide quantitative comparisons of IRdye800CW-albumin (21.6 nmol/kg) depositions in the brain and other organs when administered alone and along with HAV6 and ADTC5 peptides (13 μmol/kg). FIG. 17A provides quantitative comparisons of albumin brain depositions in pmol/g brain for control, HAV6-, and ADTC5-treated mice. FIG. 17B provides comparisons of albumin depositions in various organs using tissue NIRF signal intensities. Asterisk (*) symbol was used to indicate a significant difference with p<0.05. Error bars were used as the mean±SE for all groups.

FIGS. 18A-18B provide quantitative comparisons of IRdye800CW-IgG mAb (21.6 nmol/kg) depositions in the brain and other organs when administered alone and along with HAV6 and ADTC5 peptides (13 μmol/kg). FIG. 18A provides quantitative comparisons of IgG mAb brain depositions in pmol/g brain for control, HAV6-, and ADTC5-treated mice. FIG. 18B provides comparisons of IgG mAb depositions in various organs using tissue NIRF signal intensities. A significant difference was designated using asterisk (*) with p<0.05. The mean±SE was used for error bars.

FIGS. 19A-19B provide quantitative comparisons of IRdye800CW-fibronectin (21.6 nmol/kg) depositions in the brain and other organs when administered alone and along with ADTC5 peptide (13 μmol/kg). FIG. 19A provides NIRF intensities of brain homogenates from ADTC5-treated and control mice. FIG. 19B provides Comparisons of fibronectin depositions in of various organs using tissue NIRF signal intensities. Asterisk (*) implied a statistical significance difference between two groups with p<0.05. The mean±SE was utilized in the error bars.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, or B and C.”

As used herein, the term “amino acid” is used to refer to any organic molecule that contains at least one amino group and at least one carboxyl group where the at least one amino group is at the a position relative to the carboxyl group, where the amino acid is in the L-configuration. Naturally occurring amino acids include, for example, the twenty most common levorotatory (L,) amino acids normally found in mammalian proteins, i.e., alanine (Ala), arginine (Arg), asparagine (Asn), aspartic acid (Asp), cysteine (Cys), glutamine (Gin), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val). Naturally-occurring amino acids are those encoded by the genetic code, as well as those amino acids that are later modified, e.g., hydroxyproline, γ-carboxyglutamate, and O-phosphoserine. Amino acids may be referred to herein by either their commonly known three letter symbols or by the one-letter symbols recommended by the IUPAC-IUB Biochemical Nomenclature Commission.

As used herein, the terms “polypeptide,” “polyamino acid,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g. Na⁺, Li⁺, K⁺, Ca²⁺, Mg²⁺, Zn⁺) ammonia or organic amines (e.g. dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

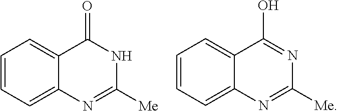

As another example, guanidines may exhibit the following isomeric forms in protic organic solution (e.g., water), also referred to as tautomers of each other:

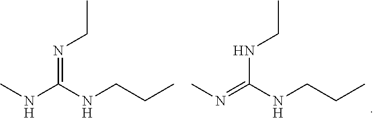

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

Throughout this disclosure, various publications, patents, and published patent specifications are referenced by an identifying citation. The disclosures of these publications, patents, and published patent specifications are hereby incorporated by reference into the present disclosure. Definitions that are contained in text incorporated by reference are excluded to the extent that they contradict definitions in this disclosure.

### The Present Technology

Delivering therapeutic and diagnostic agents across the blood-brain barrier (BBB) represents a major challenge in the diagnosis and treatment of brain diseases such as Alzheimer's disease (AD), multiple sclerosis (MS), and brain tumors. The BBB blocks passage of 98% of available drugs into the brain, and BBB efflux pumps (e.g., P-glycoproteins or Pgp) recognize and exclude even small-molecule cancer drugs and diagnostic agents In addition, while proteins have been used successfully to treat tumors or other diseases outside the brain, their physicochemical properties prevent them from readily crossing the BBB.

Treating brain tumors (e.g., glioblastoma, medulloblastoma) can be particularly difficult because the BBB blocks delivery of anti-tumor agents, mAbs, and antibody-drug conjugates (ADCs) that have been successfully used to treat tumors outside the brain. In addition, many small-molecule anti-tumor drugs such as daunomycin, doxorubicin, and adenanthin cannot treat brain tumors because they are effluxed by Pgp on the BBB.

The BBB also makes neurodegenerative diseases such as MS and AD difficult to treat. In MS, neurodegeneration is caused by immune cells that infiltrate the brain and damage the myelin sheaths surrounding neuronal axons. The extent of axonal damage correlates with the degree of disability in MS patients. Currently available drugs for MS suppress the immune response and prevent brain infiltration of immune cells to halt disease progression, but cannot reverse the neuronal damage. The repertoire of drugs available to treat MS and AD is limited, and many drug candidates, including mAbs, have failed in clinical trials.

Delivering molecules to the central nervous system (CNS), that can repair demyelination and/or neuronal damage, has the potential to reverse MS. Monoclonal antibodies (mAbs) such as anti-Nogo-A, anti-LINGO-1 (opicinumab), sHIgM22, and VX15/2503 (pepinemab) have been developed for inducing remyelination. See Ineichen, B. V.; Plattner, P. S.; Good, N.; Martin, R.; Linnebank, M.; Schwab, M. E. Nogo-A Antibodies for Progressive Multiple Sclerosis. CNS Drugs 2017, 31, (3), 187-198; Ruggieri, S.; Tortorella, C.; Gasperini, C. Anti lingo 1 (opicinumab) a new monoclonal antibody tested in relapsing remitting multiple sclerosis. Expert Rev Neurother 2017, 17, (11), 1081-1089; Ciric, B.; Howe, C. L.; Paz Soldan, M.; Warrington, A. E.; Bieber, A. J.; Van Keulen, V.; Rodriguez, M.; Pease, L. R. Human monoclonal IgM antibody promotes CNS myelin repair independent of Fc function. Brain Pathol 2003, 13, (4), 608-16; and Fisher, T. L.; Reilly, C. A.; Winter, L. A.; Pandina, T.; Jonason, A.; Scrivens, M.; Balch, L.; Bussler, H.; Torno, S.; Seils, J.; Mueller, L.; Huang, H.; Klimatcheva, E.; Howell, A.; Kirk, R.; Evans, E.; Paris, M.; Leonard, J. E.; Smith, E. S.; Zauderer, M. Generation and preclinical characterization of an antibody specific for SEMA4D. MAbs 2016, 8, (1), 150-62. Unfortunately, clinical trials for several of these mAbs, including anti-Nogo-A and anti-LINGO-1, have been terminated—Anti-LINGO-1 mAb for lack of significant therapeutic efficacy, and anti-Nogo-A for reasons that haven't been released. Similarly, mAbs to amyloid beta (Aβ) have failed to effectively treat AD. Ineffective brain delivery may have played a role in those failures. See Mullard, A. Anti-amyloid failures stack up as Alzheimer antibody flops. Nat Rev Drug Discov 2019, 10.1038/d41573-019-00064-1; and Mehta, D.; Jackson, R.; Paul, G.; Shi, J.; Sabbagh, M. Why do trials for Alzheimer's disease drugs keep failing? A discontinued drug perspective for 2010-2015. Expert Opin Investig Drugs 2017, 26, (6), 735-739.

While intracerebroventricular (ICV) administration of neuroregenerative molecules such as BDNF, nerve growth factor (NGF), and insulin-like growth factor 1 (IGF-1) can reverse neuronal damage and induce neuroregeneration in MS and AD, these molecules cannot effectively cross the BBB. Previous attempts to deliver them via the systemic circulation have met with only limited success. Since drilling a hole in the skull for ICV injection is not desirable for patients, there is an urgent need for alternative non-invasive brain-delivery methods for these promising molecules.

Thus, in summation, new technology that improves brain delivery of therapeutic and diagnostic molecules would benefit patients as well as enable scientists to study brain function and diseases in living animal models.

The present technology provides compounds, compositions, and methods that provide for delivery across the blood brain barrier of molecules that otherwise do not (or insignificantly) pass across the blood brain barrier.

Thus, in an aspect, the present technology provides a compound that is cyclo(1,6)SHAVSS (SEQ ID NO: 1; “HAVN1”) or a pharmaceutically acceptable salt thereof, cyclo(1,5)SHAVS (SEQ ID NO: 2; “HAVN2”) or a pharmaceutically acceptable salt thereof, cyclo(1,8)TPPVSHAV (SEQ ID NO: 3; “cyclic-ADTHAV”; “cyclic ADTHAV”) or a pharmaceutically acceptable salt thereof, cyclo(1,6)ADTPPV (SEQ ID NO: 4; “ADTN1”) or a pharmaceutically acceptable salt thereof, cyclo(1,5)DTPPV (SEQ ID NO: 5; “ADTN2”) or a pharmaceutically acceptable salt thereof, or acetyl-TPPVSHAV-NH₂ (SEQ ID NO: 6; “linear ADTHAV”) or a pharmaceutically acceptable salt thereof. For the sake of clarity, the structural formula of these compounds is provided below (where for the threonine residues of cyclic-ADTHAV and linear ADTHAV the configuration of the hydroxyl-bearing stereocenter, while not depicted, is R according to Cahn-Ingold-Prelog rules):

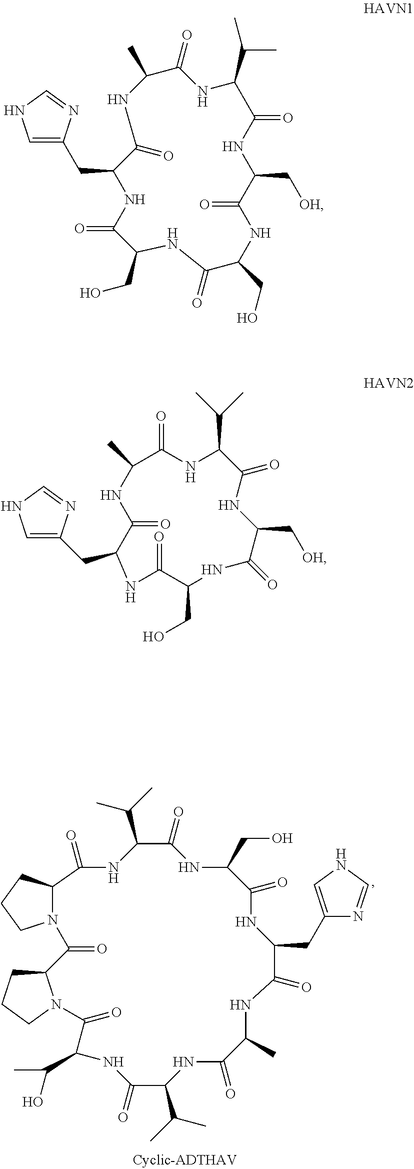 [SMILES: CC(C)[C@@H]1NC(=O)[C@H](C)NC(=O)[C@H](Cc2c[nH]cn2)NC(=O)[C@H](CO)NC(=O)[C@H](CO)NC(=O)[C@H](CO)NC1=O ; CC(C)[C@@H]1NC(=O)[C@H](C)NC(=O)[C@H](Cc2c[nH]cn2)NC(=O)[C@H](CO)NC(=O)[C@H](CO)NC1=O ; CC(C)[C@@H]1NC(=O)[C@H](C)NC(=O)[C@H](Cc2c[nH]cn2)NC(=O)[C@H](CO)NC(=O)[C@H](C(C)C)NC(=O)[C@@H]2CCCN2C(=O)[C@@H]2CCCN2C(=O)[C@H](C(C)O)NC1=O]

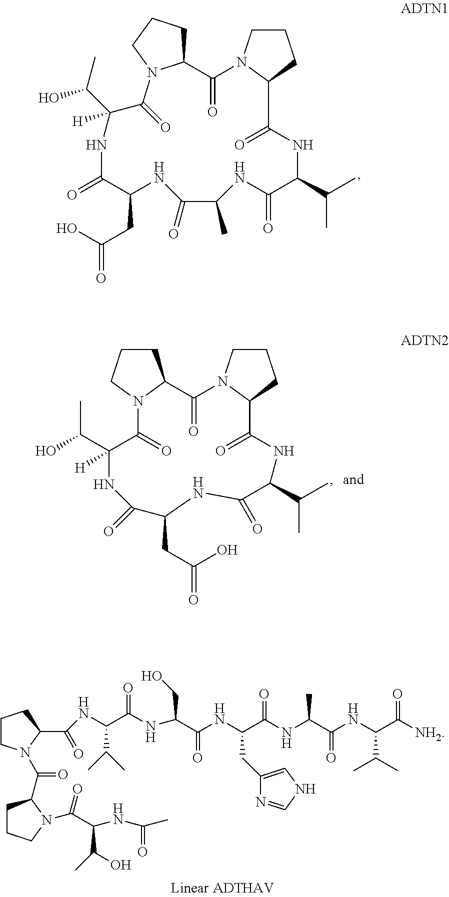 [SMILES: CC(C)[C@@H]1NC(=O)[C@@H]2CCCN2C(=O)[C@@H]2CCCN2C(=O)[C@H]([C@@H](C)O)NC(=O)[C@H](CC(=O)O)NC(=O)[C@H](C)NC1=O ; CC(C)[C@@H]1NC(=O)[C@@H]2CCCN2C(=O)[C@@H]2CCCN2C(=O)[C@H]([C@@H](C)O)NC(=O)[C@H](CC(=O)O)NC1=O ; CC(=O)N[C@H](C(=O)N1CCC[C@H]1C(=O)N1CCC[C@H]1C(=O)N[C@H](C(=O)N[C@@H](CO)C(=O)N[C@@H](Cc1c[nH]cn1)C(=O)N[C@@H](C)C(=O)N[C@H](C(N)=O)C(C)C)C(C)C)C(C)O]

These compounds of the present technology provide for delivery across the blood brain barrier (“BBB”) of compounds that otherwise do not pass across the blood brain barrier and/or are recognized and excluded by BBB efflux pumps. Such as delivery by compounds of the present technology across the BBB includes delivery of small-molecule drugs (i.e., a therapeutic compound less than 600 Daltons; e.g., adenanthin, daunomycin, doxorubicin, camptothecin, or a combination of any two or more thereof), neuroregenerative molecules (e.g., brain-derived neurotrophic factor, nerve growth factor, insulin-like growth factor 1, or a combination of any two or more thereof), medium-length peptides (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), and large proteins (e.g., a lysozyme, a ApoE2 protein, albumin, an antibody such as an antibody-drug conjugate, or a combination of any two or more thereof), as further illustrated in the Examples.

In a related aspect of the present technology, a composition is provided that includes a pharmaceutically acceptable carrier, excipient, filler, or agent (collectively referred to as “pharmaceutically acceptable carrier” unless otherwise indicated and/or specified) and one or more of HAVN1, HAVN2, cyclic-ADTHAV, ADTN1, ADTN2, linear ADTHAV, and a pharmaceutically acceptable salt of any one or more thereof. In a related aspect, pharmaceutical compositions and medicaments are provided that include an effective amount of one or more of HAVN1, HAVN2, cyclic-ADTHAV, ADTN1, ADTN2, linear ADTHAV, and a pharmaceutically acceptable salt of any one or more thereof as well as include a pharmaceutically acceptable carrier, wherein the effective amount is effective for one or more of treating a brain disease, imaging a brain disease, and diagnosing a brain disease. In a further related aspect, a method is provided that includes administering one or more of HAVN1, HAVN2, cyclic-ADTHAV, ADTN1, ADTN2, linear ADTHAV, and a pharmaceutically acceptable salt of any one or more thereof to a subject suffering from a brain disease. In a further related aspect, a method is provided that includes administering a pharmaceutical composition or medicament to a subject suffering from a brain disease, where the pharmaceutical composition or medicament includes an effective amount of one or more of HAVN1, HAVN2, cyclic-ADTHAV, ADTN1, ADTN2, linear ADTHAV, and a pharmaceutically acceptable salt of any one or more thereof as well as include a pharmaceutically acceptable carrier, wherein the effective amount is effective for one or more of treating a brain disease, imaging a brain disease, and diagnosing a brain disease.

In an aspect, a pharmaceutical composition comprising a pharmaceutically acceptable carrier and an effective amount of one or more of acetyl-SHAVSS-NH₂ (SEQ ID NO: 7; “HAV6”) or a pharmaceutically acceptable salt thereof, cyclo(1,7)acetyl-CDTPPVC-NH₂ (SEQ ID NO: 8; “ADTC5”) or a pharmaceutically acceptable salt thereof, acetyl-SHAVAS-NH₂ (SEQ ID NO: 9; “HAV4”) or a pharmaceutically acceptable salt thereof, and cyclo(1,6)acetyl-CSHAVC-NH₂ (SEQ ID NO: 10; “cHAVc3”) or a pharmaceutically acceptable salt thereof, wherein the effective amount is effective for one or more of treating a brain disease, imaging a brain disease, and diagnosing a brain disease. For the sake of clarity, the structures of HAV6, ADTC5, HAV4, and cHAVc3 are provided below (where for the threonine residue of ADTC5 the configuration of the hydroxyl-bearing stereocenter, while not depicted, is R according to Cahn-Ingold-Prelog rules):

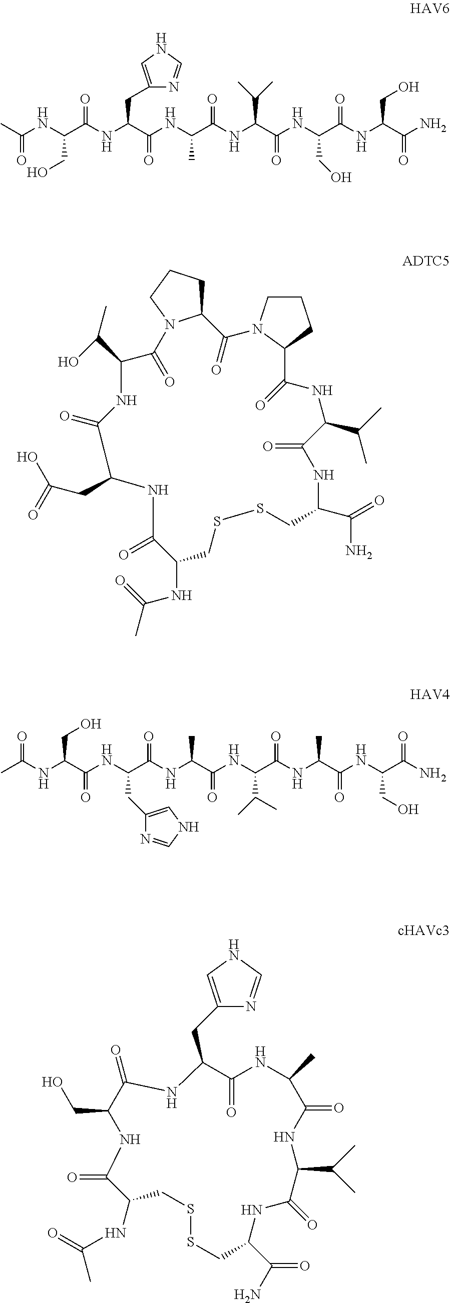

In a related aspect, a method is provided that includes administering to a subject suffering from a brain disease one or more of HAV6, ADTC5, HAV4, cHAVc3, and a pharmaceutically acceptable salt of any one or more thereof. In a further related aspect, a method is provided that includes administering to a subject suffering from a brain disease a pharmaceutical composition where the pharmaceutical composition includes an effective amount of one or more of HAV6, ADTC5, HAV4, cHAVc3, and a pharmaceutically acceptable salt of any one or more thereof as well as include a pharmaceutically acceptable carrier, wherein the effective amount is effective for one or more of treating a brain disease, imaging a brain disease, and diagnosing a brain disease.

For ease of reference, the compounds included in any aspect or embodiment herein may be referred to anywhere in this disclosure as “a compound of the present technology,” “a peptide of the present technology,” “compounds of the present technology,” or the like. Similarly for ease of reference, the compositions, medicaments, and pharmaceutical compositions of the present technology may collectively be referred to herein as “compositions” or “compositions of the present technology.”

In any embodiment and/or aspect disclosed herein (for simplicity's sake, hereinafter recited as “in any embodiment disclosed herein” or the like), the effective amount may be determined in relation to a subject. As used herein, a “subject” or “patient” is a mammal, such as a cat, dog, rodent or primate. Typically the subject is a human, and, preferably, a human suffering from or suspected of suffering from a brain disease. The term “subject” and “patient” can be used interchangeably. “Effective amount” refers to the amount of a compound or composition required to produce a desired effect. One non-limiting example of an effective amount includes amounts or dosages that yield acceptable toxicity and bioavailability levels for therapeutic (pharmaceutical) use including, but not limited to, the treatment, imaging, diagnosis (or a combination of any two or more thereof) of a brain disease, such as a brain tumor (e.g., glioblastoma, medulloblastoma), Alzheimer's disease, multiple sclerosis, and/or Parkinson's disease. Another non-limiting example of effective amount may be an amount effective in treating a brain tumor (e.g., glioblastoma, medulloblastoma) and/or shrinking a brain tumor (e.g., glioblastoma, medulloblastoma). Another non-limiting example of an effective amount includes amounts or dosages that are capable of reducing or ameliorating symptoms associated with Alzheimer's disease, multiple sclerosis, and/or Parkinson's disease. Non-limiting examples of symptoms associated with Alzheimer's disease, multiple sclerosis, and/or Parkinson's disease include mental decline, difficulty thinking and understanding, confusion in the evening hours, delusion, disorientation, forgetfulness, making things up, mental confusion, difficulty concentrating, inability to create new memories, inability to do simple math, or inability to recognize common things, tremor, seizure, depression, hallucinations, paranoia, jumbled speech, lack of appetite, difficulty with movement, weakness, or any other symptom disclosed herein. As another non-limiting example, progression or onset of Alzheimer's disease, multiple sclerosis, and/or Parkinson's disease may be slowed, halted, or reversed over a defined time period following administration of an effective amount of compound and/or composition of the present technology, as measured by a medically-recognized technique; and/or the subject with Alzheimer's disease, multiple sclerosis, and/or Parkinson's disease may be positively impacted by administration of a compound and/or composition of the present technology, as measured by a medically-recognized technique. The effective amount may be from about 0.01 μg to about 500 mg of the compound per gram of the composition, and preferably from about 0.1 μg to about 100 mg of the compound per gram of the composition. As another example, the effective amount of a compound of the present technology may be (in terms of mass of the compound/mass of patient) from 1×10⁻⁵ g/kg to 1 g/kg, 1×10⁻³ g/kg to 1.0 g/kg, 0.01 mg/kg to 100 mg/kg or, preferably, from 0.1 mg/kg to 60 mg/kg—thus, in any embodiment disclosed herein, the effective amount a compound of the present technology may be about 0.01 mg/kg, about 0.1 mg/kg, about 0.2 mg/kg, about 0.3 mg/kg, about 0.4 mg/kg, about 0.5 mg/kg, about 0.6 mg/kg/about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or any range including and/or in between any two of these values (such as, e.g., about 0.2 mg/kg to about 5 mg/kg).

In any embodiment disclosed herein, a composition of the present technology may further include a diagnostic agent and/or a therapeutic agent, such as an effective amount of the diagnostic agent and/or an effective amount of the therapeutic agent. In any embodiment disclosed herein, the diagnostic agent and/or a therapeutic agent may be a small-molecule drug (i.e., a therapeutic compound less than 600 Daltons; e.g., adenanthin, daunomycin, doxorubicin, camptothecin, or a combination of any two or more thereof), a neuroregenerative molecule (e.g., brain-derived neurotrophic factor, nerve growth factor, insulin-like growth factor 1, or a combination of any two or more thereof), a medium-length peptide (e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), a large protein (e.g., a lysozyme, a ApoE2 protein, albumin, an antibody (such as an antibody-drug conjugate), or a combination of any two or more thereof), or a combination of any two or more thereof. Useful antibodies include those antibodies listed in Table A as well as antigen-binding fragments of such antibodies and any equivalent embodiments, as would be known to those of ordinary skill in the art.

TABLE A
Representative Antibodies

| Antibody | Disclosed In |
|---|---|
| (Trade Name(s)) | (U.S. Patent or Patent Appl. Publ. No.)* |
| Belimumab | 7,138,501 |
| (BENLYSTA) | |
| Mogamulizumab | 6,989,145 |
| (POTELIGEO) | |
| Blinatumomab | 7,112,324 |
| (BLINCYTO) | |
| Ibritumomab tiuxetan | 5,776,456 |
| (ZEVALIN) | |
| Obinutuzumab | 6,602,684 |
| (GAZYVA) | |
| Ofatumumab¹ | 8,529,902 |
| (ARZERRA) | |
| Rituximab | 5,736,137 |
| (RITUXAN, MABTHERA) | |
| Inotuzumab ozogamicin | 8,153,768 |
| (BESPONSA) | |
| Moxetumomab pasudotox | 8,809,502 |
| (LUMOXITI) | |
| Brentuximab vedotin | 7,829,531; 7,090,843 |
| (ADCETRIS) | |
| Daratumumab | 7,829,673 |
| (DARZALEX) | |
| Ipilimumab | 6,984,720 |
| (YERVOY) | |
| Cetuximab | 6,217,866 |
| (ERBITUX) | |
| Necitumumab | 7,598,350 |
| (PORTRAZZA) | |
| Panitumumab | 6,235,883 |
| (VECTIBIX) | |
| Dinutuximab² | 7,432,357 |
| (UNITUXIN) | |
| Pertuzumab | 7,862,817 |
| (PERJETA, OMNITARG) | |
| Trastuzumab³ | 5,821,337 |
| (HERCEPTIN) | |
| Trastuzumab emtansine | 7,097,840 |
| (KADCYLA) | |
| Siltuximab | 7,612,182 |
| (SYLVANT) | |
| Cemiplimab⁴ | 9,987,500 |
| (LIBTAYO) | |
| Nivolumab | 8,008,449 |
| (OPDIVO) | |
| Pembrolizumab | 8,354,509 |
| (KEYTRUDA) | |
| Olaratumab | 8,128,929 |
| (LARTRUVO) | |
| Atezolizumab | 8,217,149 |
| (TECENTRIQ) | |
| Avelumab⁵ | 9,624,298 |
| (BAVENCIO) | |
| Durvalumab | 8,779,108 |
| (IMFINZI) | |
| Capromab pendetide | 5,162,504 |
| (PROSTASCINT) | |
| Elotuzumab | 7,709,610 |
| (EMPLICITI) | |
| Denosumab | 6,740,522 |
| (PROLIA, XGEVA) | |
| Ziv-aflibercept | 7,070,959 |
| (ZALTRAP) | |
| Bevacizumab | 6,054,297 |
| (AVASTIN) | |
| Ramucirumab | 7,498,414 |
| (CYRAMZA) | |
| Tositumomab | 6,565,827; 6,287,537;,6,090,365; |
| (BEXXAR) | 6,015,542; 5,843,398; 5,595,721 |
| Gemtuzumab ozogamicin | 5,773,001 |
| (MYLOTARG) | |
| Alemtuzumab | 6,569,430; 5,846,534 |
| (CAMPATH-1H) | |
| Cixutumumab | 7,968,093; 7,638,605 |
| Girentuximab | 8,466,263 |
| (RENCAREX) | |
| Nimotuzumab | 6,506,883 |
| (THERACIM, THERALOC) | |
| Catumaxomab | 9,017,676; 8,663,638; |
| (REMOVAB) | 2013/0309234A1 |
| Etaracizumab | 2004/0001835A1 |
| (ABEGRIN, VITAXIN) | |

*Note:
the disclosures of the each of the patents and patent publications listed in Table A are incorporated herein by reference.
¹Also designated 2F2.
²Also designated Ch14.18.
³Also designated HuMaB4D5-8.
⁴Also designated H4H7798N.
⁵Also designated A09-246-2.

Thus, in any embodiment disclosed herein, the diagnostic agent and/or a therapeutic agent may be one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503.

In any embodiment disclosed herein, a molar ratio of a compound of the present technology to a diagnostic agent of any embodiment disclosed herein (in a composition of the present technology and/or in a method of the present technology) may be from about 5:1 to about 3,000:1—thus, the molar ratio of any embodiment disclosed herein may be about 5:1, about 6:1, about 7:1, about 8:1, about 9:1, about 10:1, about 15:1, about 20:1, about 25:1, about 30:1, about 35:1, about 40:1, about 45:1, about 50:1, about 60:1, about 70:1, about 80:1, about 90:1, about 100:1, about 125:1, about 150:1, about 175:1, about 200:1, about 300:1, about 400:1, about 500:1, about 600:1, about 700:1, about 800:1, about 900:1, about 1,000:1, about 1,100:1, about 1,200:1, about 1,300:1, about 1,400:1, about 1,500:1, about 1,600:1, about 1,700:1, about 1,800:1, about 1,900:1, about 2,000:1, about 2,100:1, about 2,200:1, about 2,300:1, about 2,400:1, about 2,500:1, about 2,600:1, about 2,700:1, about 2,800:1, about 2,900:1, about 3,000:1, or any range including and/or in between any two of these values (such as, e.g., about 175:1 to about 2,300:1).

In any embodiment disclosed herein, a molar ratio of a compound of the present technology to a therapeutic agent of any embodiment disclosed herein (in a composition of the present technology and/or in a method of the present technology) may be from about 5:1 to about 3,000:1—thus, the molar ratio of any embodiment disclosed herein may be about 5:1, about 6:1, about 7:1, about 8:1, about 9:1, about 10:1, about 15:1, about 20:1, about 25:1, about 30:1, about 35:1, about 40:1, about 45:1, about 50:1, about 60:1, about 70:1, about 80:1, about 90:1, about 100:1, about 125:1, about 150:1, about 175:1, about 200:1, about 300:1, about 400:1, about 500:1, about 600:1, about 700:1, about 800:1, about 900:1, about 1,000:1, about 1,100:1, about 1,200:1, about 1,300:1, about 1,400:1, about 1,500:1, about 1,600:1, about 1,700:1, about 1,800:1, about 1,900:1, about 2,000:1, about 2,100:1, about 2,200:1, about 2,300:1, about 2,400:1, about 2,500:1, about 2,600:1, about 2,700:1, about 2,800:1, about 2,900:1, about 3,000:1, or any range including and/or in between any two of these values (such as, e.g., about 175:1 to about 2,300:1).

In any embodiment of the present technology, the pharmaceutical composition may be packaged in unit dosage form. The unit dosage form is effective in treating, imaging, diagnosing (or a combination of any two or more thereof) a brain disease. Generally, a unit dosage including a compound of the present technology will vary depending on patient considerations. Such considerations include, for example, age, protocol, condition, sex, extent of disease, contraindications, concomitant therapies and the like. An exemplary unit dosage based on these considerations may also be adjusted or modified by a physician skilled in the art. For example, a unit dosage for a patient comprising a compound of the present technology may vary from 1×10⁻⁵ g/kg to 1 g/kg (mass of the compound/mass of patient), preferably 1×10⁻³ g/kg to 1.0 g/kg. Dosage of a compound of the present technology may also vary from 0.01 mg/kg to 100 mg/kg or, preferably, from 0.1 mg/kg to 60 mg/kg. Thus, in any embodiment disclosed, a compound of the present technology may be included at a dosage of about 0.01 mg/kg, about 0.1 mg/kg, about 0.2 mg/kg, about 0.3 mg/kg, about 0.4 mg/kg, about 0.5 mg/kg, about 0.6 mg/kg/about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or any range including and/or in between any two of these values. Suitable unit dosage forms, include, but are not limited to powders, tablets, pills, capsules, lozenges, suppositories, patches, nasal sprays, injectables, implantable sustained-release formulations, mucoadherent films, topical varnishes, lipid complexes, etc.

The pharmaceutical compositions and medicaments may be prepared by mixing one or more peptides of the present technology with pharmaceutically acceptable carriers, excipients, binders, diluents or the like in order to prevent, treat, image, diagnose (or a combination of any two or more thereof) a brain disease. The peptides and compositions described herein may be used to prepare formulations and medicaments that treat a brain disease. Such compositions may be in the form of, for example, granules, powders, tablets, capsules, syrup, suppositories, injections, emulsions, elixirs, suspensions or solutions. The instant compositions may be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, and/or emulsifying agents may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms often include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), and “Remington: The Science and Practice of Pharmacy,” 20^{th} Edition, Editor: Alfonso R Gennaro, Lippincott, Williams & Wilkins, Baltimore (2000), each of which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also include, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Various assays and model systems can be readily employed to determine the therapeutic effectiveness of the treatment according to the present technology.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

In any embodiment disclosed herein of a method of the present technology, the method may ameliorate at least one symptom selected from (a) a symptom from the Integrated Alzheimer's Disease Rating Scale (iADRS) selected from the group consisting of personal belonging management, selection of clothes, ability to dress self, ability to clean habitation, financial management ability, writing ability, ability to keep appointments, ability to use telephone, ability to prepare food for self, travel ability, awareness of current events, reading ability, interest in television, ability to shop for self, ability to remain alone, ability to perform chores, ability to perform a hobby or game, driving ability, self-management of medications, ability to initiate and finish complex tasks, and ability to initiate and finish simple tasks; (b) a sign from the Alzheimer's Disease Assessment Scale-Cognitive subscale (ADAS-Cog) selected from the group consisting of learning, naming, command following, ideational praxis, constructional praxis, orientation, and recognition memory; (c) a symptom from the Alzheimer's Disease Cooperative Study—instrumental Activities of Daily Living (ADCS-iADL) wherein the symptom is any of the symptoms recited in (a) or (b); (d) constipation; (e) depression; (f) cognitive impairment; (g) short or long term memory impairment; (h) concentration impairment; (i) coordination impairment; (j) mobility impairment; (k) speech impairment; (l) mental confusion; (m) sleep problem, sleep disorder, or sleep disturbance; (n) circadian rhythm dysfunction; (o) REM disturbed sleep; (p) REM behavior disorder; (q) hallucinations; (r) fatigue; (s) apathy; (t) erectile dysfunction; (u) mood swings; (v) urinary incontinence; or (w) neurodegeneration.

Amelioration of a symptom is measured using a clinically recognized scale or tool. Further, the amelioration of the symptom may be, for example, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale or test, for example, any of those described herein. In any embodiment disclosed herein, amelioration of the symptom or treatment of Alzheimer's disease, multiple sclerosis, and/or Parkinson's disease may be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis. In any embodiment disclosed herein, progression or onset of Alzheimer's disease, multiple sclerosis, and/or Parkinson's disease may be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique, via administration of a compound and/or composition of the present technology.

As disclosed vide supra, in any embodiment disclosed herein of a method of the present technology, the effective amount of a compound of the present technology may be about 0.01 mg/kg, about 0.1 mg/kg, about 0.2 mg/kg, about 0.3 mg/kg, about 0.4 mg/kg, about 0.5 mg/kg, about 0.6 mg/kg/about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or any range including and/or in between any two of these values (such as, e.g., about 0.2 mg/kg to about 5 mg/kg).

In any embodiment disclosed herein of a method of the present technology, a compound and/or composition of any embodiment herein of the present technology may be administered in combination with a diagnostic agent and/or a therapeutic agent, and may be administered in combination with an effective amount of the diagnostic agent and/or an effective amount of the therapeutic agent. Such a diagnostic agent and/or a therapeutic agent may be administered (a) concomitantly; (b) as an admixture; (c) separately and simultaneously or concurrently; or (d) separately and sequentially, with respect to the compound and/or composition of the present technology. In any embodiment herein, the diagnostic agent and/or a therapeutic agent may be a small-molecule drug (i.e., a therapeutic compound less than 600 Daltons; e.g., adenanthin, daunomycin, doxorubicin, camptothecin, or a combination of any two or more thereof), a neuroregenerative molecule (e.g., brain-derived neurotrophic factor, nerve growth factor, insulin-like growth factor 1, or a combination of any two or more thereof), a medium-length peptide (e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), a large protein (e.g., a lysozyme, a ApoE2 protein, albumin, an antibody (such as an antibody-drug conjugate), or a combination of any two or more thereof), or a combination of any two or more thereof. In any embodiment herein, the diagnostic agent and/or a therapeutic agent may be one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503.

As disclosed vide supra, in any embodiment disclosed herein of a method of the present technology a molar ratio of a compound of the present technology to a diagnostic agent of any embodiment disclosed herein may be from about 5:1 to about 3,000:1—thus, the molar ratio of any embodiment disclosed herein may be about 5:1, about 6:1, about 7:1, about 8:1, about 9:1, about 10:1, about 15:1, about 20:1, about 25:1, about 30:1, about 35:1, about 40:1, about 45:1, about 50:1, about 60:1, about 70:1, about 80:1, about 90:1, about 100:1, about 125:1, about 150:1, about 175:1, about 200:1, about 300:1, about 400:1, about 500:1, about 600:1, about 700:1, about 800:1, about 900:1, about 1,000:1, about 1,100:1, about 1,200:1, about 1,300:1, about 1,400:1, about 1,500:1, about 1,600:1, about 1,700:1, about 1,800:1, about 1,900:1, about 2,000:1, about 2,100:1, about 2,200:1, about 2,300:1, about 2,400:1, about 2,500:1, about 2,600:1, about 2,700:1, about 2,800:1, about 2,900:1, about 3,000:1, or any range including and/or in between any two of these values (such as, e.g., about 175:1 to about 2,300:1).

Also as disclosed vide supra, in any embodiment disclosed herein of a method of the present technology a molar ratio of a compound of the present technology to a therapeutic agent of any embodiment disclosed herein may be from about 5:1 to about 3,000:1—thus, the molar ratio of any embodiment disclosed herein may be about 5:1, about 6:1, about 7:1, about 8:1, about 9:1, about 10:1, about 15:1, about 20:1, about 25:1, about 30:1, about 35:1, about 40:1, about 45:1, about 50:1, about 60:1, about 70:1, about 80:1, about 90:1, about 100:1, about 125:1, about 150:1, about 175:1, about 200:1, about 300:1, about 400:1, about 500:1, about 600:1, about 700:1, about 800:1, about 900:1, about 1,000:1, about 1,100:1, about 1,200:1, about 1,300:1, about 1,400:1, about 1,500:1, about 1,600:1, about 1,700:1, about 1,800:1, about 1,900:1, about 2,000:1, about 2,100:1, about 2,200:1, about 2,300:1, about 2,400:1, about 2,500:1, about 2,600:1, about 2,700:1, about 2,800:1, about 2,900:1, about 3,000:1, or any range including and/or in between any two of these values (such as, e.g., about 175:1 to about 2,300:1).

In any embodiment disclosed herein of a method of the present technology, it may be the method does not include intracerebroventricular injection of a compound and/or composition of the present technology. In any embodiment disclosed herein of a method of the present technology, it may be the method does not include the method does not comprise intracerebroventricular injection.

The examples herein are provided to illustrate advantages of the present technology and to further assist a person of ordinary skill in the art with preparing or using the compounds and compositions of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects, or embodiments of the present technology.

### EXAMPLES

### Representative In Vivo Brain Delivery of Monoclonal Antibody Using Compounds of the Present Technology

Materials and Methods: The reagents (e.g., trifluoro acetic acid (TFA), hydrogen gas, Pd/C catalyst, triisopropylsilane (TIPS), hexafluorophosphate azabenzotriazole tetramethyl uronium (HATU), diisopropyl ethyl amine (DIEA)) and solvents (e.g., acetonitrile, methanol) were purchased from Sigma Aldrich Chemical Company (St. Louis, Mo.) and Fisher Scientific Inc. (Hampton, N.H.). Gyros Protein Technologies Inc. (Tucson, Ariz.) was the vendor for all Fmoc-protected amino acids for peptide synthesis. IRDye800CW donkey anti-goat IgG was obtained from LI-COR Inc. (Lincoln, Nebr.). All animal studies were carried out under the approved animal protocol granted by Institutional Animal Care and Use Committee (IACUC) at The University of Kansas. Animal Care Unit (ACU) personnel and veterinarians were involved in the care of the animals used in this study.

### Peptide Synthesis and Purification

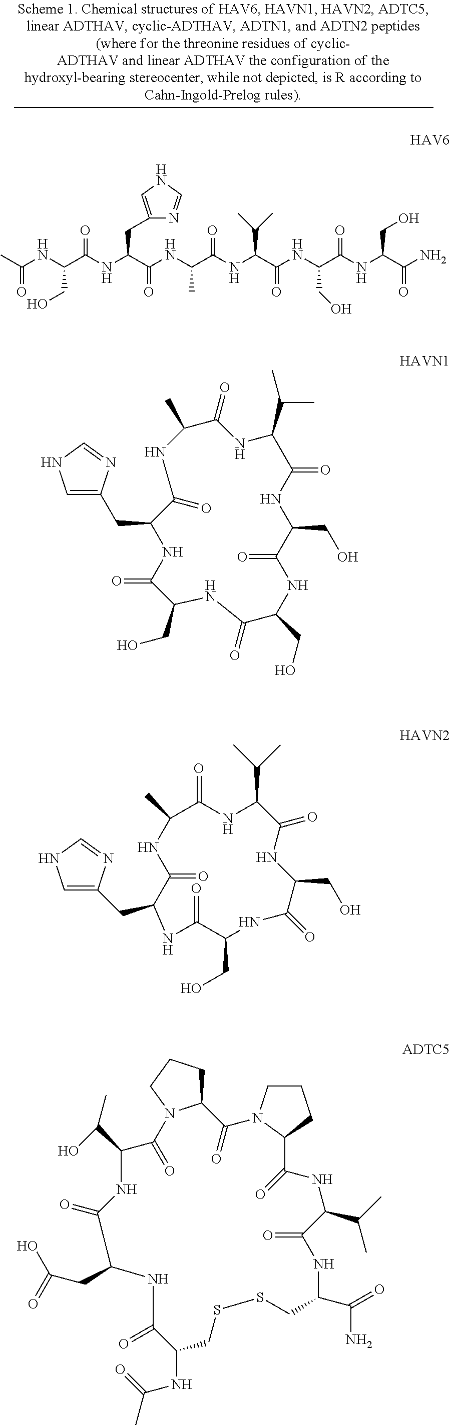 [SMILES: CC(=O)N[C@@H](CO)C(=O)N[C@@H](Cc1c[nH]cn1)C(=O)N[C@@H](C)C(=O)N[C@H](C(=O)N[C@@H](CO)C(=O)N[C@@H](CO)C(N)=O)C(C)C ; CC(C)[C@@H]1NC(=O)[C@H](C)NC(=O)[C@H](Cc2c[nH]cn2)NC(=O)[C@H](CO)NC(=O)[C@H](CO)NC(=O)[C@H](CO)NC1=O ; CC(C)[C@@H]1NC(=O)[C@H](C)NC(=O)[C@H](Cc2c[nH]cn2)NC(=O)[C@H](CO)NC(=O)[C@H](CO)NC1=O ; CC(=O)N[C@H]1CSSC[C@@H](C(N)=O)NC(=O)[C@H](C(C)C)NC(=O)[C@@H]2CCCN2C(=O)[C@@H]2CCCN2C(=O)[C@H](C(C)O)NC(=O)[C@H](CC(=O)O)NC1=O]

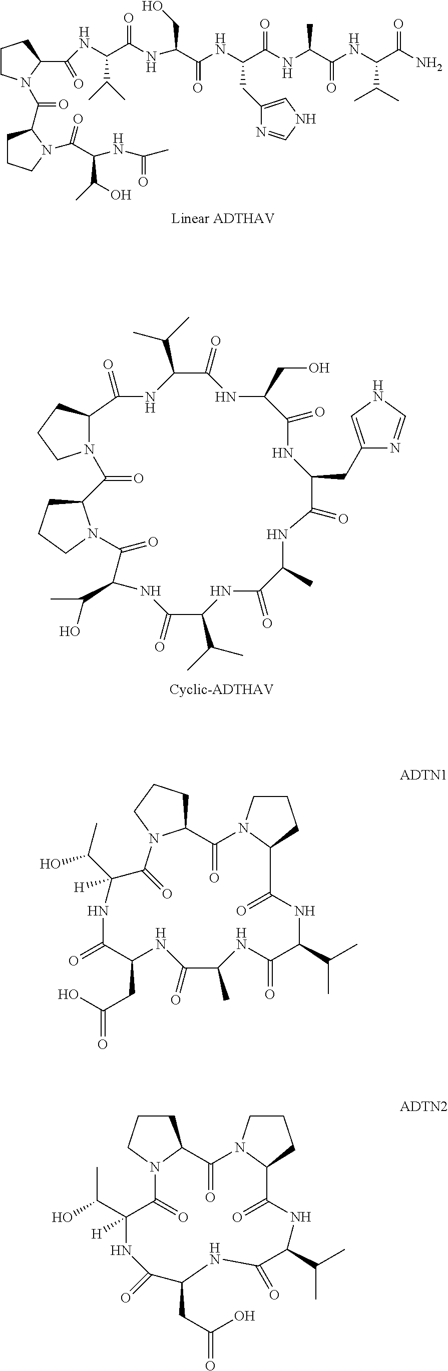 [SMILES: CC(=O)N[C@H](C(=O)N1CCC[C@H]1C(=O)N1CCC[C@H]1C(=O)N[C@H](C(=O)N[C@@H](CO)C(=O)N[C@@H](Cc1c[nH]cn1)C(=O)N[C@@H](C)C(=O)N[C@H](C(N)=O)C(C)C)C(C)C)C(C)O ; CC(C)[C@@H]1NC(=O)[C@H](C)NC(=O)[C@H](Cc2c[nH]cn2)NC(=O)[C@H](CO)NC(=O)[C@H](C(C)C)NC(=O)[C@@H]2CCCN2C(=O)[C@@H]2CCCN2C(=O)[C@H](C(C)O)NC1=O ; CC(C)[C@@H]1NC(=O)[C@@H]2CCCN2C(=O)[C@@H]2CCCN2C(=O)[C@H]([C@@H](C)O)NC(=O)[C@H](CC(=O)O)NC(=O)[C@H](C)NC1=O ; CC(C)[C@@H]1NC(=O)[C@@H]2CCCN2C(=O)[C@@H]2CCCN2C(=O)[C@H]([C@@H](C)O)NC(=O)[C@H](CC(=O)O)NC1=O]

A Tribute solid-phase peptide synthesizer (Gyros Protein Technologies, Inc., Tucson, Ariz.) with Fmoc chemistry was used to synthesize all linear peptide precursors. The HAV6 and linear precursors for ADTC5 were synthesized using amide resin and were cleaved from the resin with a cocktail mixture of 89% TFA:5% phenol:3% H₂O:3% TIPS. The linear precursors for N-to-C-termini cyclic peptides (i.e., HAVN1, HAVN2, cyclic-ADTHAV, ADTN1, and ADTN2) were synthesized using Fmoc-Val-Wang resin (see Scheme 2). The carboxylic acid and alcohol groups on the side chains were protected with benzyl groups. The peptides were cleaved using a 94% TFA: 3% H₂O: 3% TIPS cocktail solution. The TFA solutions of linear HAV6, ADTC5, and ADTHAV were added into cold diethyl ether to precipitate the peptide. In contrast, the cleavage solutions of linear HAVN1 and HAVN2 were directly concentrated by rotary evaporator to yield the crude peptides that were further lyophilized.

To form ADTC5, a very low concentration of linear peptide precursor without any protecting groups was dissolved in bicarbonate buffer solution at pH 9.0; and then, the solution was then bubbled with air to oxidize the two thiol groups in the Cys residues to form a disulfide bond. The end result produced ADTC5 peptide in a monomeric form with low side products as dimers, trimers, and oligomers. The desired monomer was purified by semi-preparative HPLC using a C18 column Waters) (Bridge C18 (19 mm×250 mm, 5 μm particle size; Waters Corporation, Milford, Mass.). The mobile phase consisted of solvents (A) H₂O: ACN: TFA (94.9:5:0.1) and (B) acetonitrile with a gradient of 40% B (0 min), 40-100% B (17 min), 100% B (2 min), 100-40% B (2 min), and 40% B (6 min). Before combining the collected fractions, each fraction was evaluated by analytical HPLC using a C18 column (Luna C18, 4.6 mm×250 mm, 5 ,um particle size, 100 A; Phenomenex, Inc., Torrance, Calif.) to check for purity, and the pure fractions were pooled, concentrated, and lyophilized.

The N-to-C-termini cyclizations to produce cyclic-ADTHAV, HAVN1, HAVN2, ADTN1, and ADTN2 were carried out in solution phase (see Scheme 2). The acid and alcohol functional groups on the side chains of the peptide were protected with benzyl ester and ether groups that were removed after cyclization. The optimized molar ratio of peptide: HATU: DIEA for the cyclization reaction was 1:2:4, and the cyclization reaction was done in dilute solution (˜6.0 mM peptide) in acetonitrile (ACN). In this case, three separate solutions were prepared: (1) 6.3 mmol peptide in 50 mL of acetonitrile, (2) 12.6 mmol HATU in 50 mL acetonitrile, and (3) 25.2 mmol DIEA in 1 L of acetonitrile. The solutions of peptide and HATU were both added slowly from two different peristaltic pumps into the DIEA solution over 4 h, and the mixture was stirred overnight. The completion time for the cyclization reaction was monitored using mass spectrometry every 4 h to observe the disappearance of the linear precursor and the appearance of the cyclic peptide. After confirming the complete formation of the cyclic peptide, the acetonitrile was removed by rotary evaporator. A C18 semi-preparative HPLC column was used to isolate the cyclic peptide, and the pure peptide was lyophilized. The cyclic peptide was dissolved in methanol and subjected to hydrogenation reaction under balloon pressure in the presence of Pd/C catalyst overnight to remove benzyl ester and ether protecting groups. The final product was purified by semi-preparative HPLC, and the identity of the cyclic peptide was confirmed using mass spectrometry.

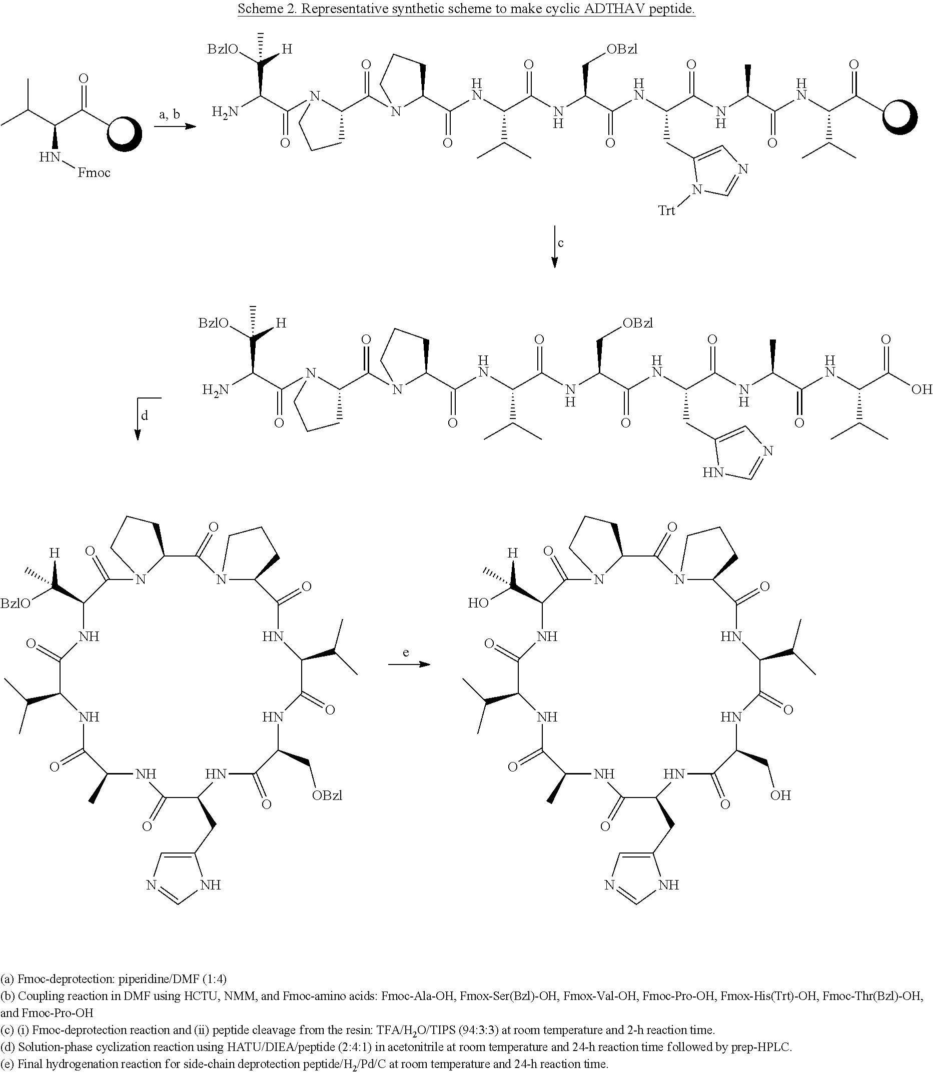

### In Vivo Delivery of IRdye800CW IgG mAb

The activity of each peptide in enhancing blood-brain barrier (BBB) penetration was evaluated by delivering IRdye800CW donkey anti-goat IgG mAb in C57BL/6 mice; the amounts of mAb in the brain were determined using NIRF imaging. Each group contained 3 mice per group (n=3) with a mixture of male and female mice, selected randomly for each arm of the study. The injection solution was prepared by adding 600 μL PBS into 0.5 mg lyophilized IgG mAb; then, approximately 1.5 mg lyophilized peptide was added into the mixture yielding the injectable formulation. A 100 μL solution of a mixture containing IgG mAb (21.6 nmol/kg) along with 13 μmol/kg peptide of the present technology was administered via tail vein. As a control, 100 μL of IgG mAb alone was administered via i.v. route. After the delivered molecules had been circulating for 15 min, the mice were sacrificed; then, a mixture of PBS with 0.5% Tween20 was administered for cardiac perfusion to remove the blood and deliver molecules into the brain microvessels. The brain and other organs such as lung, heart, spleen, liver, and kidney were harvested and rinsed with PBS. The isolated organs were scanned with Odyssey® CLx for mAb quantification.

The brain deposition of IgG mAb was also quantified by NIRF imaging in brain homogenates. The isolated brains were mechanically homogenized in 2.0 mL of PBS. To make the standard solutions, IRDye800CW IgG mAb stock solution (70 μg/mL) was prepared; it was then diluted with various amounts of PBS to make six different mAb concentrations. To generate a calibration curve, the brain homogenate (200 μL) was aliquoted out to a 96-well plate. 10 μL of each concentration of IgG mAb was added to three different wells of blank brain homogenates. The standard spiked homogenates were at a range of 10-200 ng/mL IgG mAb in brain homogenate. The wells were scanned using the Odyssey® CLx scanner, and the signal intensities vs. concentrations of mAb per gram of brain were used to generate a calibration curve.

Statistical Analysis: ANOVA with Student-Newman-Keuls was used to compare the data for determining statistical significance for IgG mAb deposition in the brains. A p-value of less than 0.05 was used as a criterion for a significant difference in data comparison.

### Results

ADTHAV, HAVN1, and HAVN2 were compared to ADTC5 and HAV6 peptides by evaluating their activities in delivering IgG mAb into the brains of C57BL/6 mice. As a negative control, IgG mAb was delivered in PBS without a peptide of the present technology. Previously, ADTC5 has been shown to improve brain delivery of IgG mAb, which can serve as a positive control. Cyclic HAV peptides (i.e., HAVN1, HAVN2) and linear HAV6 were evaluated to test whether the formation of cyclic peptides could improve their BBB modulatory activity. Cyclic ADTHAV peptide was formed via a combination of ADTC5 and HAV6 sequences to test the potential additive activity of the two sequences. Because ADTC5 and HAV6 bind to two different binding sites on the EC1 domain, it is proposed that the activity of cyclic ADTHAV is also due to its binding to two different binding sites on the EC1 domain.

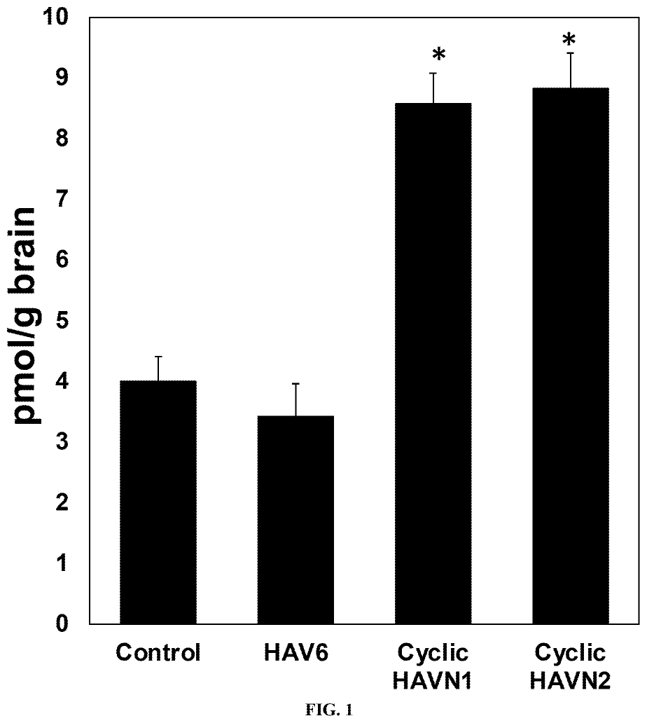

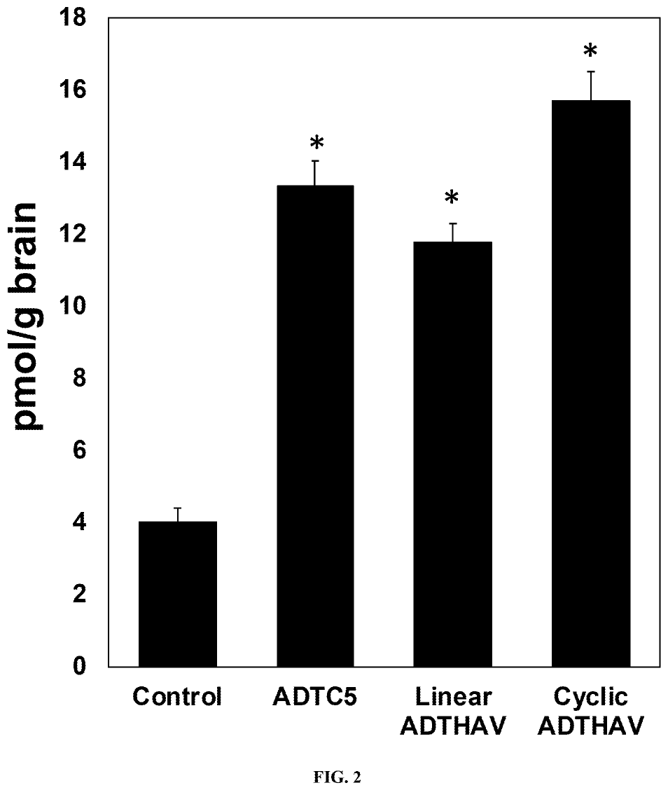

A calibration curve was generated to determine the amount of IgG mAb in the brain by spiking blank brain homogenates with a concentration range from 10 to 200 ng/mL, and good linearity with R²≥0.98 was achieved. FIG. 1 illustrates the results which showed that HAV6 did not enhance brain delivery of IgG mAb compared to control (i.e., IgG mAb alone, p>0.05) while IgG mAb brain delivery was significantly enhanced by cyclic HAVN1 and HAVN2 peptides compared to HAV6 and control. These results indicate that cyclic peptide formation increases BBB modulatory activity of HAV peptide. The average amounts of IgG mAb in the brains of HAV6-treated and control animals were 3.4±0.4 and 4.0±0.5 pmol/g brain, respectively. In contrast, the average amounts of mAb in the brains of cyclic HAVN1- and HAVN2-treated mice were 8.6±0.5 and 8.8±0.6 pmol/g brain, respectively. The BBB modulatory activities of ADTC5, linear ADTHAV, and cyclic ADTHAV were also compared to control, the results of which are illustrated in FIG. 2. The brain delivery of IgG mAb by linear ADTHAV, cyclic ADTHAV, and ADTC5 was significantly better than in the PBS control. The average brain deposition of IgG mAb were 11.8±0.5, 15.7±0.8, and 13.3±0.7 pmol/g brain for linear ADTHAV, cyclic ADTHAV, and ADTC5, respectively. It is expected that performing similar studies as described herein with ADTN1 and ADTN2 will provide results similar or significantly improved over HAVN1 and HAVN2.

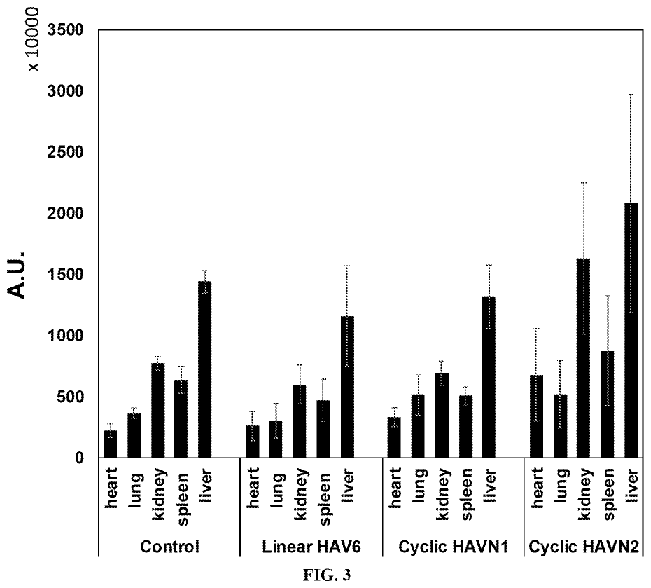

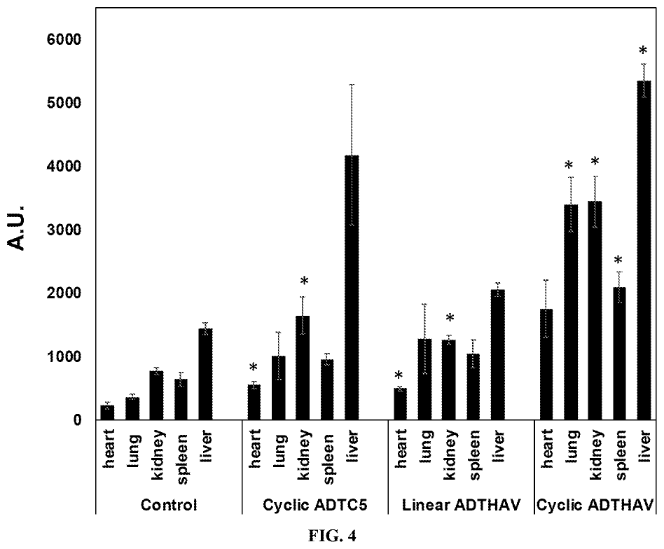

The effects of peptides of the present technology in the deposition of IgG mAb in other organs such as liver, kidney, heart, spleen, and lungs were compared to control. There was no significant difference in IgG mAb deposition in other organs for HAV6-, HAVN1- and HAVN2-treated animals compared to control animals (see FIG. 3; p>0.05). It is expected that performing similar studies as described herein with ADTN1 and ADTN2 will provide results similar or significantly improved over HAVN1 and HAVN2. Moreover, these results suggest that these BBB-modulating peptides of the present technology do not have a significant impact on other organs. In contrast, as shown in FIG. 4, ADTC5 and linear ADTHAV peptides have significant effects on the distribution of IgG mAb in the heart and kidney when compared to control. There were significant increases in deposition of IgG mAb in liver, kidney, spleen, and lungs for cyclic ADTHAV peptide when compared to control (see FIG. 4; p<0.05).

### Delivery of a Recombinant Brain-Derived Neurotrophic Factor (BDNF) to the Brains of Healthy and Experimental Autoimmune Encephalomyelitis (EAE) Mice

In this study, BDNF (13 kDa monomer) was delivered to the brains of relapsing-remitting experimental autoimmune encephalomyelitis (RR-EAE) mice using ADTC5 peptide via i.v. administrations to induce remyelination and neurorepair as a less invasive method compared to intracerebroventricular (ICV) injection. Four different groups of EAE mice were treated eight times with BDNF+ADTC5, BDNF alone, ADTC5 alone or vehicle during the remission period of EAE. Therapeutic effects of delivering BDNF in vivo were evaluated by observing the amelioration of EAE relapse and comparing clinical body scores across treatment groups. Finally, the effects of BDNF in the brains of EAE mice were evaluated using several ex vivo analyses to indicate remyelination and the degree of NG2-glia activity as well as by probing mRNA transcript upregulation of proteins affected by BDNF. It is expected that performing similar studies as described herein with HAVN1, HAVN2, ADTN1, ADTN2, and/or cyclic ADTHAV will provide results that are similar or significantly improved.

### Materials and Methods

Animals: The protocols to use live mice have been approved by the Institutional Animal Care and Use Committee (IACUC) at The University of Kansas. SJL/elite mice were purchased from Charles River Laboratories, Inc. (Wilmington, Mass.). All mice were housed under specific pathogen-free conditions at the animal facility at The University of Kansas approved by the university Animal Care Unit (ACU). The animals were maintained in the Animal Care Unit with free access to food, water, and rotating stimuli.

Peptide Synthesis and Purification: The syntheses of the ADTC5 and PLP139-151 (amino acid residues 139 to 151 of myelin proteolipid protein) peptides were accomplished using a solid-phase peptide synthesizer (Gyros Protein Technologies, Tucson, AZ). After peptide cleavage from the resin using TFA, the crude peptides were precipitated overnight in cold diethyl ether. In most cases, the crude precipitate showed high concentrations of the desired peptide. The formation of a disulfide bond in the cyclic peptide (i.e., ADTC5) was accomplished by vigorously stirring the precursor linear peptide in bicarbonate buffer solution under air oxidation at pH 9.0 in high dilution. The cyclization reaction produced primarily the desired monomer with minor oligomer side products; the monomer peptide was isolated from the mixture using a semi-preparative HPLC X-bridge C18 column (Waters, Milford, Mass.). After purification with semi-preparative HPLC, the isolated peptides had high purity (>95%) as determined by analytical HPLC. The exact mass of each peptide was determined by mass spectrometry.

EAE Mouse Model: EAE disease in animals (5-8-week-old SJL/elite female mice, Charles River) was stimulated by injecting 200 μg of PLP₁₃₉₋₁₅₁ peptide in a 0.2 mL emulsion containing equal volumes of PBS and complete Freund's adjuvant (CFA) with killed mycobacterium tuberculosis strain H37RA (Difco, Detroit, Mich.; final concentration 4 mg/mL) as described in Kobayashi, N.; Kiptoo, P.; Kobayashi, H.; Ridwan, R.; Brocke, S.; Siahaan, T. J. Prophylactic and therapeutic suppression of experimental autoimmune encephalomyelitis by a novel bifunctional peptide inhibitor. Clin Immunol 2008, 129, (1), 69-79 and Kobayashi, N.; Kobayashi, H.; Gu, L.; Malefyt, T.; Siahaan, T. J. Antigen-specific suppression of experimental autoimmune encephalomyelitis by a novel bifunctional peptide inhibitor. J Pharmacol Exp Ther 2007, 322, (2), 879-86. Briefly, 50 pL of PLP/CFA emulsion was administered to four different regions above the shoulders and the flanks on Day 0 followed by intraperitoneal injection of 200 ng of pertussis toxin (List Biological Laboratories, Campbell, Calif.) on Days 0 and 2. Clinical scores that reflect the disease progression were determined using an 11-point scale with 0.5 increments ranging from 0 to 5; 0 being no apparent disease and 5 being moribund. On Day 21, mice were randomly separated into 3 treatment groups: (i) BDNF (5.7 nmol/kg)+ADTC5 (10 μmol/kg; n=7), (ii) BDNF alone (5.71 nmol/kg, n=6), (iii) ADTC5 alone (10 μmol/kg; n=5), and (iv) vehicle (n=5). All mice received 8 intravenous injections every 4 days beginning on Day 21. The mice were euthanized via CO₂ inhalation on Day 55. Area under the curve (AUC) calculations were used to compare clinical scores across groups; AUC calculations were performed using the trapezoid rule from Days 21 to 55.

Euthanasia, Brain Perfusion, and Extraction: All mice were euthanized via a CO₂ chamber. Immediately following euthanasia, mice underwent cervical dislocation and were transcardially perfused with PBS+0.2% Tween-20 followed by perfusion-fixation with a 4% paraformaldehyde and 30% sucrose PBS solution. Following the fixation, the brains were extracted and post-fixed overnight in the perfusion-fixation solution.

### Immunohistochemistry

Fixed brain samples were submitted to IHC World (Ellicott City, Md.) for paraffin embedding, tissue sectioning (5 μm), anti-NG2 (Abcam, Cambridge, UK) staining via DAB, and Luxol-fast blue staining. Staining protocols described on the IHC World website for Luxol-fast blue and immunohistochemistry enzyme HRP were performed. For both procedures, brains were cut into 5 μm sections and then deparaffinized and rehydrated using xylenes and an ethanol-water gradient. For Luxol staining, sections were incubated in Luxol-fast blue solution at 56° C. overnight and subsequently rinsed with 95% ethyl alcohol followed by distilled water. For anti-NG2 mAb staining, sections underwent antigen retrieval, followed by rinsing with PBS-Tween 20 for 2×2 minutes. Sections were incubated with normal serum block followed by primary antibody incubation with anti-NG2 mAb at 4° C. overnight and subsequently rinsed with PBS-Tween 20. Sections were then blocked using a peroxidase blocking solution for 10 min at room temperature (RT). Next, samples were incubated with a biotinylated secondary antibody at 1-10,000 dilution in PBS for 30 min at RT. Sections were then incubated in streptavidin-HRP in PBS for 30 min at RT followed by incubation in DAB solution for 1-3 min. Sections were dehydrated through 95% ethanol for 2 min, 100% ethanol for 2×3 min, and cleared with xylene. Sections were mounted using aqueous mounting media and coverslipped using 1.5 coverslips.

Luxol-fast blue and anti-NG2 mAb images were taken under identical conditions on a Zeiss Axioplan 2 microscope (Oberkochen, Germany) equipped with a mercury lamp excitation source, and 40× (Luxol) and 20× (anti-NG2) air objective lenses. Greyscale images for quantification were taken using a 1344×1024 Orca ER CCD camera (Hamamatsu Photonics, Japan), color images for qualitative purposes were taken using a 1.3 MP Spot Color camera (Spot Imaging, Sterling Heights, Mich.). To determine the degree of demyelination (i.e., breakages in the myelin sheath), 5 greyscale images from each group were randomly selected and converted to binary, and regions of interest (ROI) were manually selected within the lateral corpus callosum using ImageJ (National Institute of Health, Bethesda, Md.). A binary value of ‘1’ (i.e., white signal) implied a lack of myelin, whereas a binary value of ‘0’ (i.e., black signal) implied myelin. The mean value of each ROI from each image was recorded. To determine the degree of anti-NG2 staining, densitometry analysis was performed on DAB stained sections; greyscale images were taken under equal exposure times and 5 images per group were randomly selected and used for analysis. ROIs of identical size were selected within the medial corpus callosum. The integrated mean grey value for each ROI from each image was recorded. Staining background was controlled for by subtracted an aggregate of mean grey values from 5 ROIs of negative controls from each group.

### Fluorescent In Situ Hybridization

Coronal brain sections (5 μm thickness) from mid- and hind-brain were sectioned and washed three times in PBS before mounting on gelatin-coated glass slides (Superfrost Plus, Thermo Fisher Scientific). Tissue was allowed to dry at RT and then stored at −20° C. until use. Fluorescent in situ hybridization (FISH) was performed using RNAscope® Technology 2.0, Advanced Cell Diagnostics (ACD), Hayward, Calif.) Multiplex Reagent Kit V2. See Vasquez, J. J.; Hussien, R.; Aguilar-Rodriguez, B.; Junger, H.; Dobi, D.; Henrich, T. J.; Thanh, C.; Gibson, E.; Hogan, L. E.; McCune, J.; Hunt, P. W.; Stoddart, C. A.; Laszik, Z. G. Elucidating the Burden of HIV in Tissues Using Multiplexed Immunofluorescence and In Situ Hybridization: Methods for the Single-Cell Phenotypic Characterization of Cells Harboring HIV In Situ. J Histochem Cytochem 2018, 66, (6), 427-446; Gershon, T. R.; Crowther, A. J.; Liu, H.; Miller, C. R.; Deshmukh, M. Cerebellar granule neuron progenitors are the source of Hk2 in the postnatal cerebellum. Cancer Metab 2013, 1, (1), 15; and Smith, P. A.; Schmid, C.; Zurbruegg, S.; Jivkov, M.; Doelemeyer, A.; Theil, D.; Dubost, V.; Beckmann, N. Fingolimod inhibits brain atrophy and promotes brain-derived neurotrophic factor in an animal model of multiple sclerosis. J Neuroimmunol 2018, 318, 103-113. In short, mounted tissue sections were deparaffinized using xylene and serially dehydrated in 50%, 70%, 95%, and 100% ethanol for 5 min each. In between all pretreatment steps, tissue sections were briefly washed with nanopure water. Pretreatment solution 1 (hydrogen peroxide reagent) was applied for 10 min at RT and then the tissue sections were boiled in pretreatment solution 2 (target retrieval reagent) for 15 min. Mounted slices were pretreated with solution 3 (protease reagent) for 30 min at 40° C. in the HybEz™ hybridization system (ACD). Following tissue pretreatment, the following transcript probes were applied to all sections: Mm-EGR1-C1 (Cat. #423371), Mm-NOS1-C2 (Cat. #437651-C2), and Mm-ARC-C3 (Cat. #316911-C3), which correspond to early growth response 1 (EGR1), nitric oxide synthase 1 (NOS1), and activity-related cytoskeleton-associated protein (ARC). Probes were hybridized to sections for 2 hours (h) at 40° C. and then subsequently washed for 2 min at room temperature. Following hybridization, hybridize AMP 1 was applied to each slide, which was then incubated for 30 min at 40° C. The same process was repeated for hybridize AMP 2 and 3. For HRP-C1 signal development (EGR1), HRP-C1 was applied to each slide, which was incubated for 15 min at 40° C. and then washed. For C1, TSA® Plus fluorescein (Perkin Elmer, Akron, Ohio) was applied and incubated for 30 min at 40° C. and then washed. Following the wash, HRP blocker was applied to each slide, which was incubated for 15 min at 40° C. and then washed. This process was repeated for C2 (NOS1), and C3 (EGR1) using TSA® Plus Cy3 and Cy5, respectively. The resulting transcript-fluorophore labeling is as follows: EGR1-fluorescein, NOS1-Cy3, EGR1-Cy5. All sections were counterstained by incubating DAPI for 30 seconds (sec) at RT following by rinsing. Slides were then covered using ProLong Gold Antifade Mountant and 1.5 coverslips. Slides were allowed to dry in the dark overnight at 4° C. All sections were imaged within 2 weeks.

Fluorescent images were taken using an Olympus Inverted Epifluorescence Microscope XI81 (Olympus Life Solutions, Waltham, Mass.) running SlideBook Version 5.5 (3i, Ringsby, Conn.) equipped with a digital CMOS camera (2000×2000), automatic XYZ stage position, ZDC autofocus, and a xenon lamp excitation source. Images were taken using a 20× objective and appropriate filter sets for each fluorophore (i.e., DAPI, FITC, Cy3, C5). To determine the degree of mRNA transcript expression, 5 images of analogous regions of the cerebral cortex were randomly selected from mouse samples of each group, and the total number of cells expressing each mRNA transcript were counted using ImageJ. The number of cells expressing each mRNA transcript was normalized against the total number of cells (as determined by DAPI) to ensure that analyzed areas had equal cell density. For display purposes, images were pseudo colored using ImageJ; green was assigned to fluorescein (EGR1), magenta was assigned to Cy5 (ARC), and blue to DAPI. NOS images were not incorporated due to virtually no signal detection.

### Western Blots

Female SJL/elite mice, 5 weeks of age (Charles River) were initially intravenously injected via lateral tail vein with 5.71 nmol/kg BDNF (Peprotech, Rocky Hill, N.J.) with (n=3) or without (n=3) 10 μmol/kg ADTC5. BDNF was allowed to circulate for 20-30 min prior to euthanasia via CO₂. Immediately following euthanasia, mice were transcardially perfused with protease inhibitor infused TRIS buffer (pH 7.4). The brains of the mice were extracted and placed in the perfusate buffer on ice. For Western blotting, 100-150 mg of brain tissue was sectioned from the most ventral-posterior portion of the brain and placed in 200-250 μL of solution mixture containing 66% tissue protein extraction reagent (TPER; Thermo Fisher, Waltham, Wash.) and 33% 50 μL of neural protein extraction reagent (NPER; Thermo Fisher) with protease and phosphatase inhibitors (Thermo Fisher). The tissue samples were lysed via sonication using a Sonic Disembrator 500 (Thermo Fisher) at an amplitude level of 15 Hz for a maximum of 10 sec. Following sonication, the samples were vortexed for one minute and then centrifuged at 4° C. and 13,000 RPM for 30 min. The sonication, vortexing, and centrifugation were repeated 2 times. Following lysis and centrifugation, NuPAGE™ 4-12% Bis-Tris Protein Gels (1.5 mm, 10-well, Thermo Fisher) were loaded with 60 μg of protein and Licor (Lincoln, Nebr.) loading buffer. A BDNF standard of less than 1.0 μg was also loaded for positive control. The gel was run at 100 V for 2 h. Following the gel, the protein bands were transferred to a nitrocellulose membrane (Licor) at 36 V overnight. Following the transfer, the membrane was stained with REVERT (Licor) for 3 min and then washed using the REVERT Wash Solution for 2 min followed immediately by scanning using a Licor Odyssey at 700 nm. Next, the membrane was washed using the REVERT Reversal Solution (Licor) and subsequently blocked for 2 h at 4° C. using Licor TBS blocking reagent. The membrane was then incubated with the primary antibody, anti-BDNF (Abcam), at a 1:1,000 ratio in TBS+0.1% Tween-20 for 36 h at 4° C. Following primary antibody, the membrane was rinsed and incubated with the IR800-conjugated secondary antibody (Licor) for 1.5 h at room temperature in the dark. The membrane was then immediately scanned using a Licor Odyssey CLX at a wavelength of 800 nm. Following imaging of BDNF bands on the membrane, the membrane was stripped using stripping buffer to be reprobed for the phosphorylated-TrkB (pTrkB) receptor with anti-phospho-TrkB (EMD Millipore, Burlington, Mass.) at a 1:1,000 dilution in TBS+0.1% Tween-20 for 24 h at 4° C. Following primary antibody incubation, the membrane was rinsed and incubated with the IR800-conjugated secondary antibody for 1.5 h at room temperature in the dark. The membrane was then immediately scanned using the same parameters as for the BDNF imaging. These bands were not densiometrically analyzed due to high background signal; however, they are shown for qualitative analysis.

To improve the level of detection of BDNF and pTrkB bands via Western blot, the above process was repeated with an increase in dosages of BDNF. The dosages of ADTC5 remained constant; mice received either 57.1 nmol/kg BDNF (10-fold increase)+10 μmol/kg ADTC5 (n=2), 28.6 nmol/kg BDNF (5-fold increase)+10 μmol/kg ADTC5 (n=1), or 28.6 nmol/kg BDNF alone (5-fold increase; n=3). These images were not quantified due to the variation in dosing regiments; however, they are provided for qualitative analysis of BDNF brain depositions.

Statistics: All statistics were performed using GraphPad Prism (San Diego, Calif.). Analysis of variance (ANOVA) and Student's T-test were performed when appropriate, both operating at 95% confidence intervals with a p-value of less than 0.05 used as the criterion for statistical significance unless otherwise stated.

### Results

### Effect of BDNF Brain Delivery by ADTC5 on Suppression of EAE Relapse

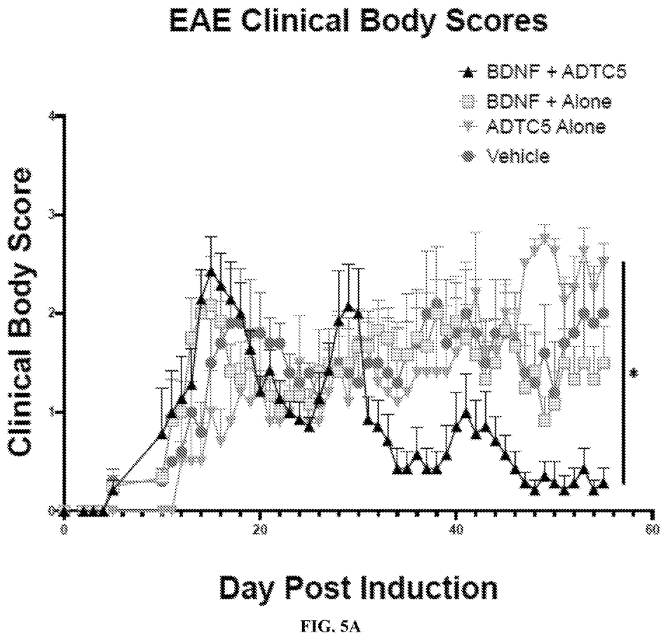

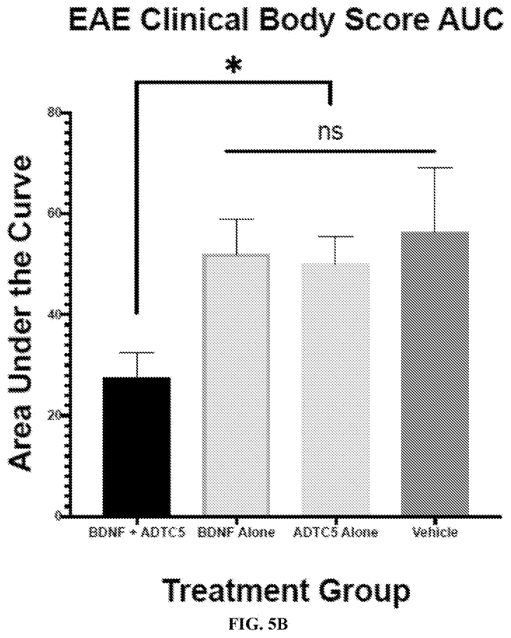

The ability of ADTC5 to deliver BDNF into the brains of mice after i.v. administrations was assessed by determining the effects of BDNF in suppressing disease relapse in the relapsing-remitting EAE animal model. The efficacy of BDNF (5.71 nmol/kg)+ADTC5 (10 μmol/kg; n=7) was compared to that of BDNF alone (5.71 nmol/kg; n=6), ADTC5 alone (10 μmol/kg; n=5), and vehicle (n=5). I.V. injections were performed every 4 days up to eight injections starting from day 21 during the time of disease remission and relapse. EAE clinical scores were monitored daily from the beginning to the end of the study. The EAE mice that received injections of BDNF+ADTC5 had clinical body scores significantly lower over time compared to the mice that received BDNF alone, ADTC5 alone or vehicle (FIG. 5A). The mice that received injections of BDNF+ADTC5 showed normal locomotion on all four limbs, with some residual tail paralysis. In contrast, mice that received BDNF alone, ADTC5 alone or vehicle showed partial or full hind leg paralysis and full tail paralysis.

The differences in clinical body scores were distinguished by generation of the areas under the curve (AUC) disease scores of all four groups from day 21 to day 55, after the peak of the disease. It was found that mice that received injections of BDNF+ADTC5 had significantly lower ACU disease scores compared to those that received BDNF alone, ADTC5 alone or vehicle (F_{(3,9)}=3.180; p≤0.05; FIG. 5B). There was no significant difference in the clinical scores between treatments with BDNF alone, ADTC5 alone and PBS (F_{(2,13)}=0.128; p=0.881). The results suggest that ADTC5 helps BDNF to penetrate the BBB to exert its biological activity in the brain while BDNF alone did not have efficacy due to its inability to penetrate the BBB. Further evaluation of the therapeutic efficacy of systemically delivered BDNF using ADTC5 peptide was assessed using histological, immunohistochemical, and hybridization methods.

### Effect of BDNF on Remyelination

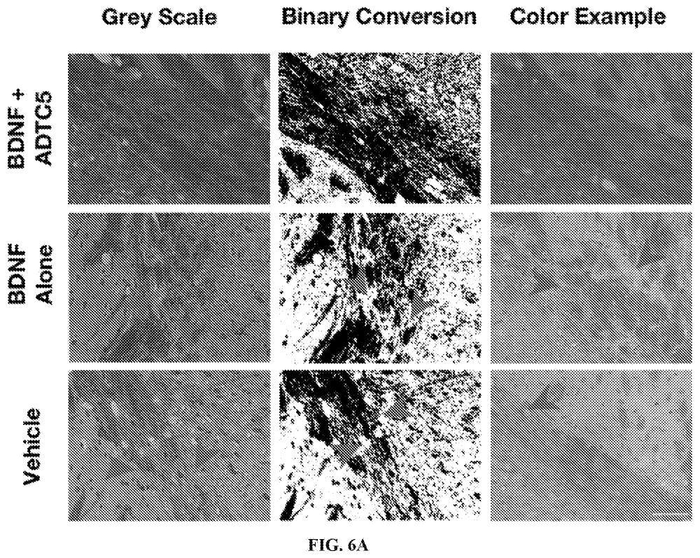

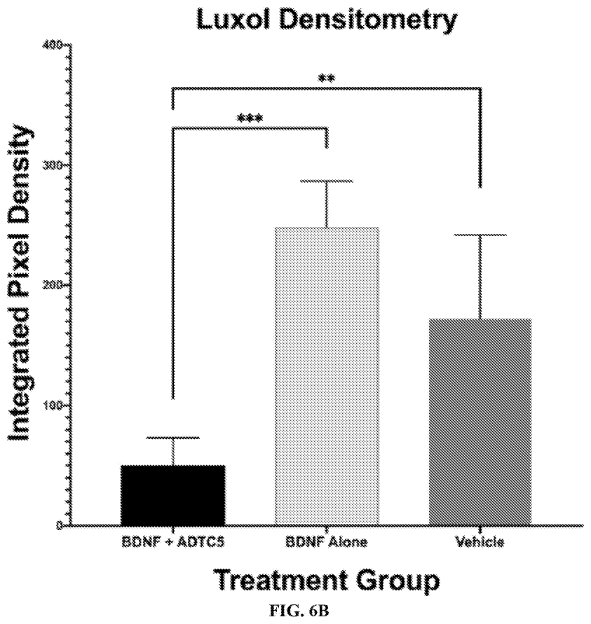

The ability of BDNF to induce remyelination has been previously demonstrated using BDNF knockout mice in which myelin loss was shown to be sensitive to a lack of BDNF expression. Additionally, BDNF has been shown to improve remyelination and regeneration of nerve fibers after C7 ventral root avulsion and replantation. Thus, myelin levels in the brains of mice were probed as an indication that BDNF is successfully entering the CNS and exerting an effect. Myelin levels in the brain were imaged using Luxol fast-blue chromogen staining. FIG. 6A shows noticeably more dense myelin staining in the lateral corpus callosum in mice that received BDNF+ADTC5 (n=5) compared to those that received BDNF alone (n=5), or vehicle (n=5). The mice that received BDNF alone or vehicle showed myelin discontinuity (white spaces) in the corpus callosum. Quantification via densitometry using binary images of myelin staining showed a statistically significant increase in myelin density in the lateral corpus for mice that received BDNF+ADTC5 compared to those that received BDNF alone or vehicle (F_{(2,12)}=21.72; p≤0.001) (FIG. 6B). This result supports that BDNF successfully entered the brain with the help of ADTC5 and induced remyelination in the corpus callosum.

### Effect of BDNF on NG2-Glia

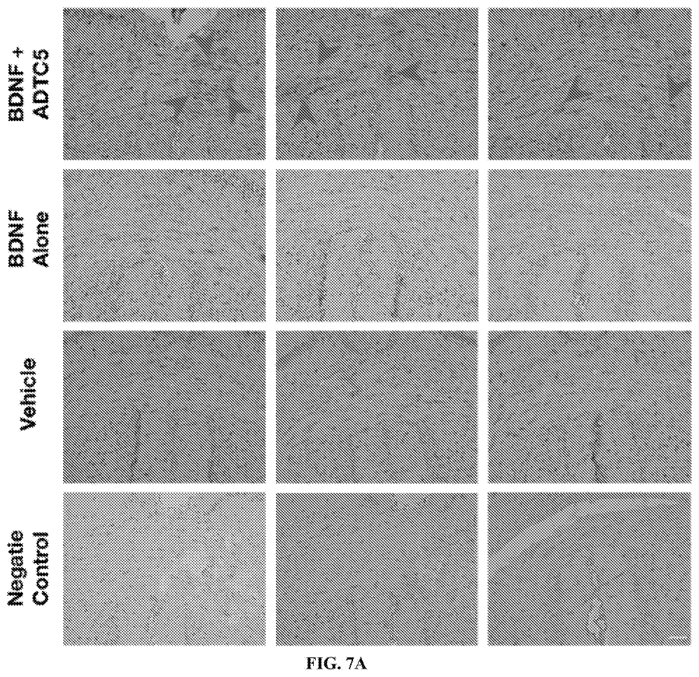

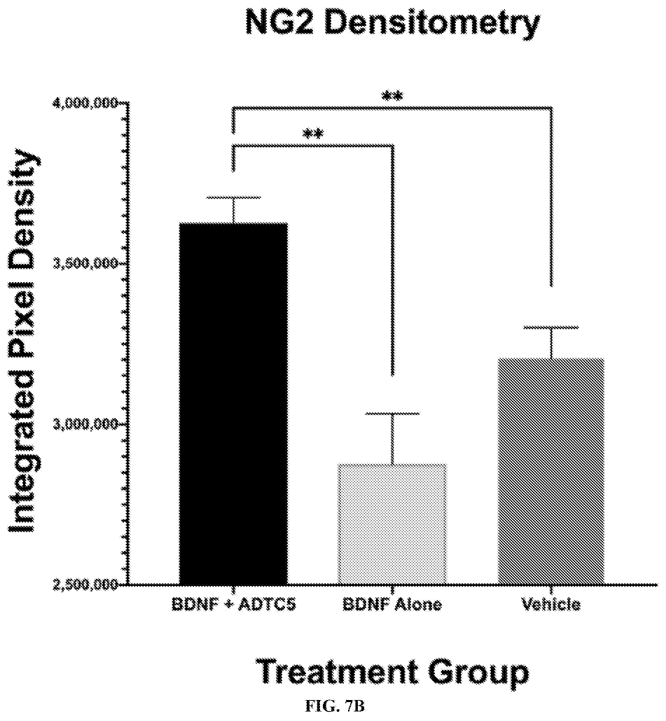

The NG2 receptors have previously been shown to facilitate the maturation of oligodendrocyte precursor cells and have been demonstrated to be distinctly upregulated in BDNF^{+/+} mice following the development of cuprizone-induced lesions. NG2 receptor presence was further probed as an additional indicator that BDNF is indeed entering the CNS and exerting a therapeutic effect. NG2 receptor levels were quantified using anti-NG2 immunohistochemistry staining. A higher degree of NG2 staining in the medial corpus callosum of mice was found in animals that received BDNF+ADTC5 (n=5) compared to those that received BDNF alone (n=5) or vehicle (n=5; FIG. 7A). Quantification of the degree of NG2 staining was determined using mean grey values. Mice that received BDNF+ADTC5 showed a significantly increased level of anti-NG2 staining compared to those that received BDNF alone or vehicle (F_{(2,12)}=10.44, p≤0.01; FIG. 7B). These results are evidence that BDNF is inducing oligodendrocyte maturation and, in turn, remyelination.

### Effects of BDNF on EGR1, ARC, and NOS1 mRNA Transcript Expression

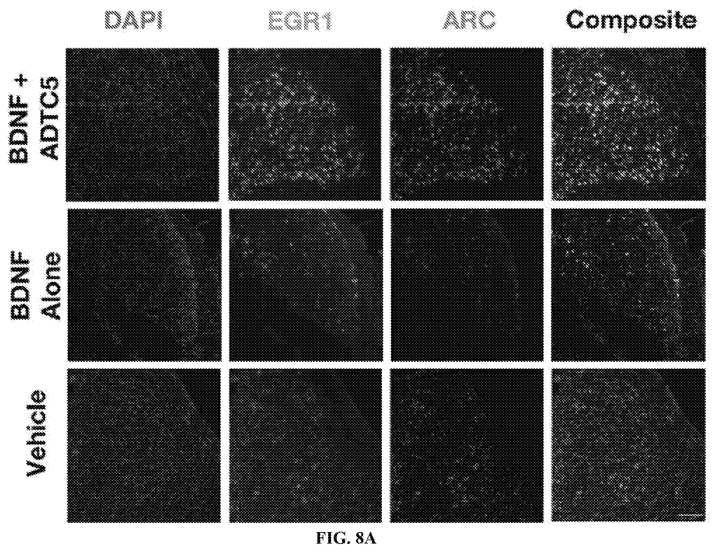

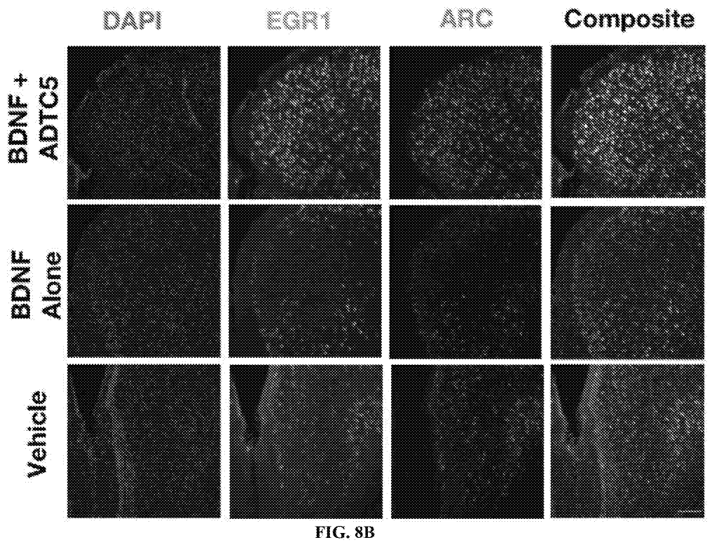

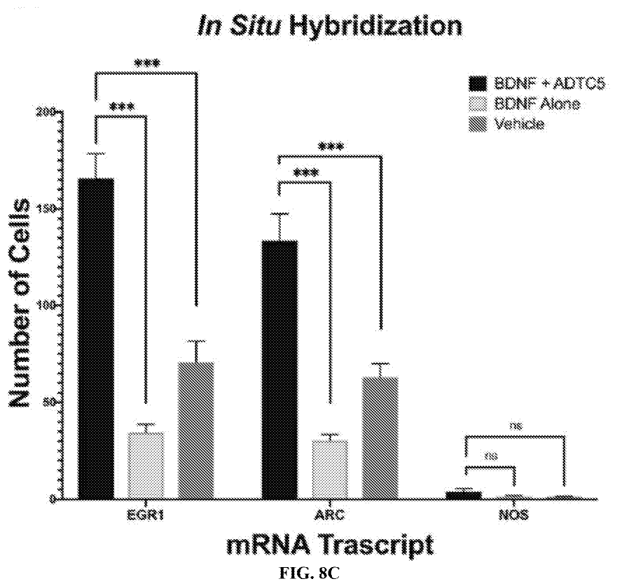

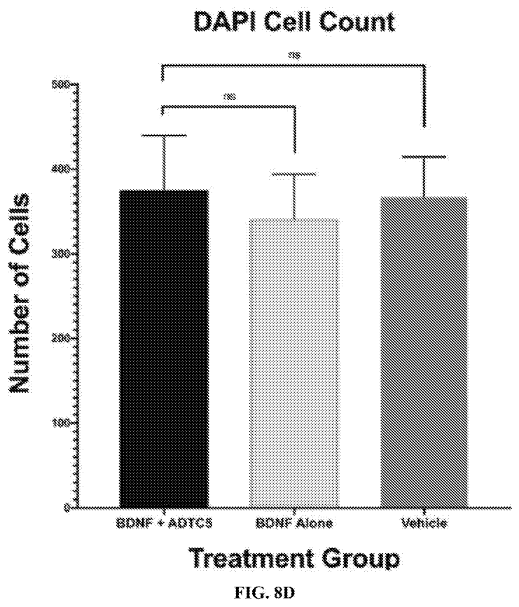

BDNF exposure is well known to affect downstream transcription factors including c-fos, cAMP response element binding protein (CREB), early growth response-1 (EGR-1), and EGR3. Furthermore, EGR1 has been demonstrated to target the activity-regulated ARC gene, and EGR1 is also upregulated by BDNF exposure. In addition, BDNF has not only been shown to upregulate specific downstream transcripts, but has also been shown to inhibit the expression of nitric oxide synthase 1 (NOS1). Therefore, we probed three mRNA transcripts, EGR1, ARC, and NOS1 for evidence that BDNF is entering the brain and exhibiting effects. The mRNA expression levels of EGR1, ARC, and NOS1 mRNA were quantified using fluorescent in situ hybridization (FISH). FIG. 8A and FIG. 8B show brain sections from the mid and hind brain, respectively; mice that received BDNF+ADTC5 have noticeable upregulation of EGR1 and ARC mRNA transcripts compared to mice that received BDNF alone or vehicle. However, images for the NOS1 mRNA expressions are not shown due to low level of detectability. The mRNA expression levels were quantified using cell counting that was normalized against the number of cell nuclei to ensure that analyzed areas were of equal cell density. Composite images of all fluorescent channels showed a pronounced increase in mRNA transcripts that can be seen for the mice that received BDNF+ADTC5 (n=5) compared to the mice that received BDNF alone (n=5) or vehicle (n=5; FIGS. 8A-8B). FIG. 8C shows a significant increase in EGR1 (F_{(2,12)}=47.10; p≤0.001) and ARC (F_{(2,12)}=33.43; p≤0.001) expression levels for mice that received BDNF+ADTC5 compared to those of the mice that received BDNF alone or vehicle. In contrast, there was no significant difference in NOS (F_{(2,12)}=1.826; p=0.203) or DAPI (F_{(2,12)}=0.504; p=0.617) staining across the three groups (FIG. 8D).

### Detection of BDNF in the Brain using Western Blots

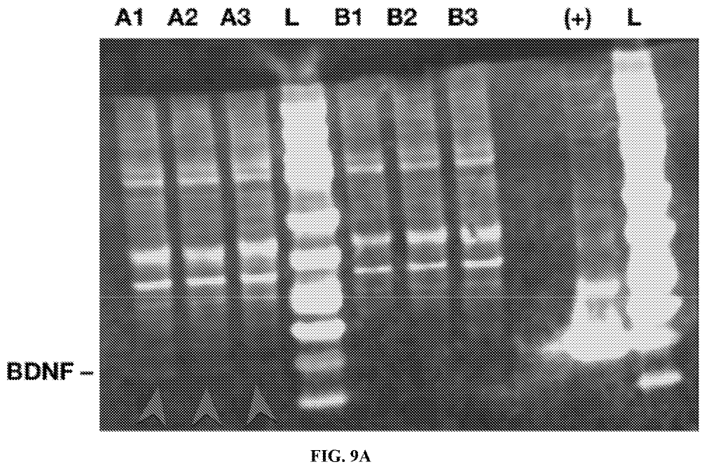

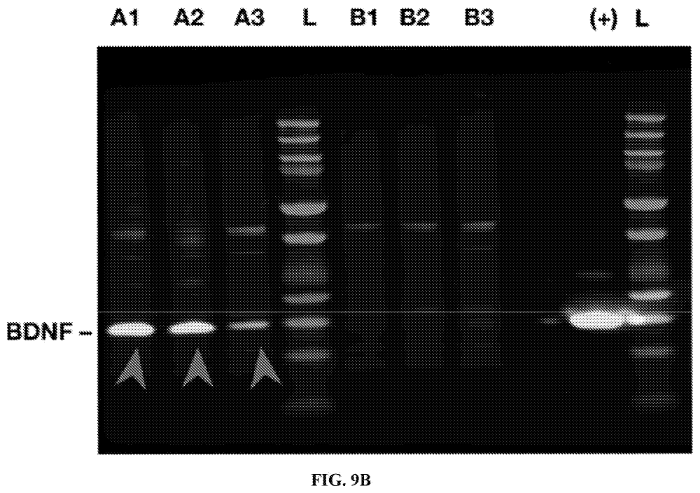

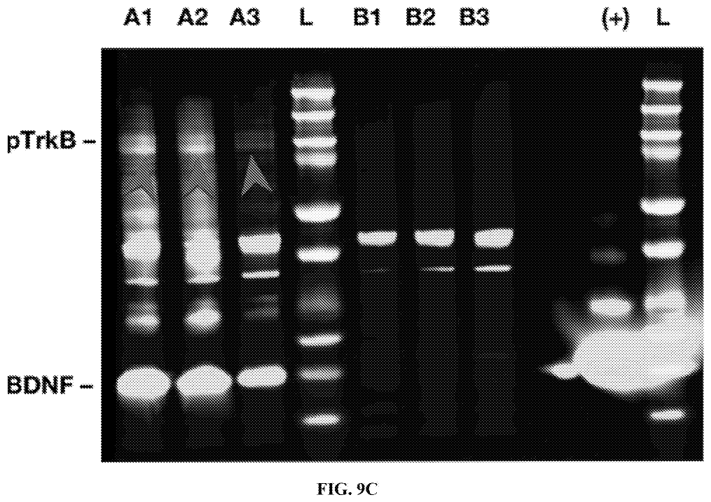

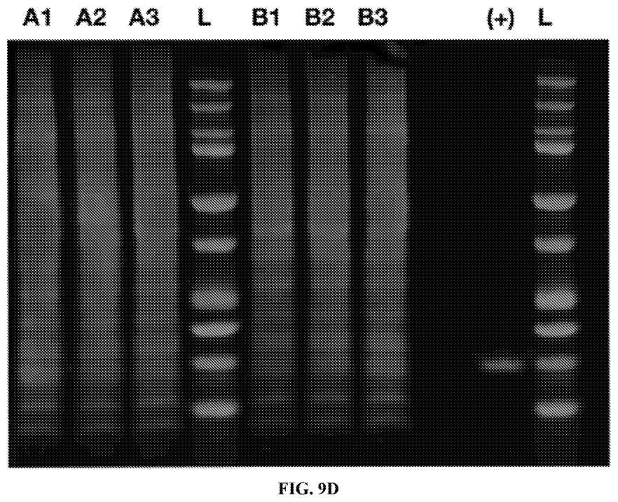

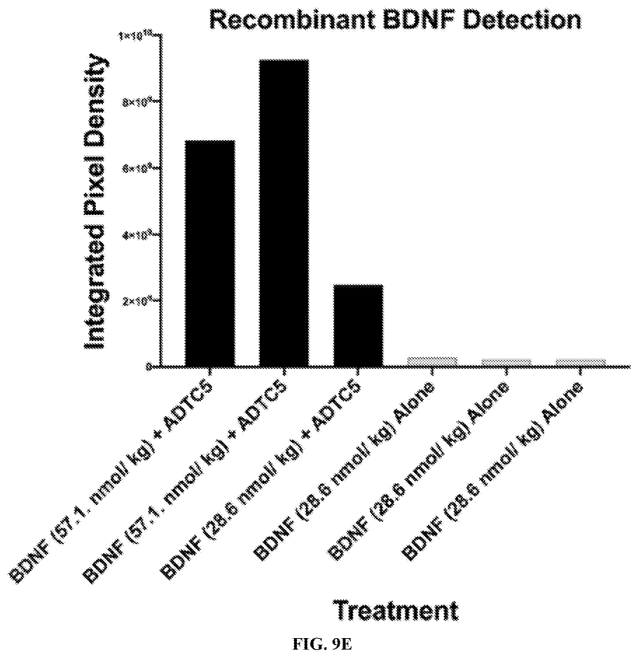

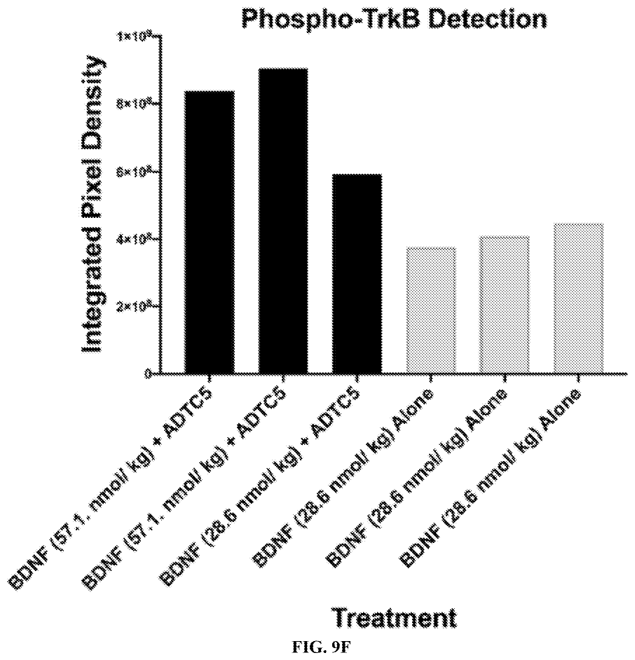

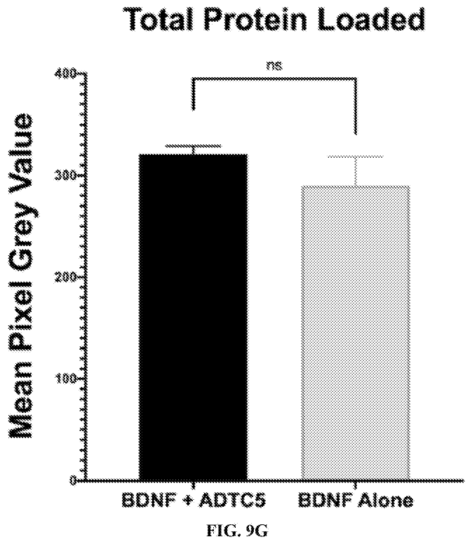

The ability of ADTC5 to deliver BDNF into the brain was confirmed by Western blot analysis of the brain homogenates. To determine if BDNF entered the brain using the ADTC5 peptide, mice were initially given a 5.71 nmol/kg BDNF injection with (n=3) or without (n=3) 10 μmol/kg ADTC5 and were sacrificed after 20 minutes to allow for sufficient circulation and activation of the pTrkB pathway. FIG. 9A shows a notable increase in detection of BDNF bands in the brains of mice that received injections of BDNF+ADTC5 compared to those that received BDNF alone, where delivered BDNF was undetected. Because of high background, pTrkB could not be detected with confidence using this Western blot.

Due to suboptimal detection of pTrkB using 5.71 nmol/kg BDNF injections, the above process was repeated with increases in dosage of BDNF to 57.1 nmol/kg but with the dosages of ADTC5 remaining constant. Mice received either 57.1 nmol/kg BDNF (10-fold increase)+10 μmol/kg ADTC5 (n=2), 28.6 nmol/kg BDNF (5-fold increase)+10 μmol/kg ADTC5 (n=1), or 28.6 nmol/kg BDNF alone (5-fold increase; n=3). FIG. 9B more clearly shows an increase in detection of BDNF and pTrkB bands for mice that were treated with BDNF+ADTC5 compared to mice that were treated with BDNF alone. Additionally, to ensure that total protein loaded into each well across all groups was consistent, a total protein stain was performed (FIG. 9C); this serves as a more reliable and accurate loading control in comparison to detecting a ubiquitous protein such as actin. There was no significant difference in the total protein loading across each group (t₍₄₎=1.808; p=0.145). Due to the variation of dosages of BDNF administered, the densiometric BDNF and pTrkB bands cannot be statistically compared with confidence; however, the relative intensities are shown in FIG. 9D. The aggregate results of these two Western blots indicate that BDNF is successfully entering the CNS and inducing an immediate effect on upregulation of pTrkB. FIG. 9E provides a graphical representation of recombinant BDNF detection level in mice that received BDNF (57.1 nmol/kg)+ADTC5 (10 μmol/kg; A1, A2), BDNF (28.6 nmol/kg)+ADTC5 (10 μmol/kg; A3), or BDNF alone (28.6 nmol/kg; B1, B2, B3). FIG. 9F provides a graphical representation of pTrkB detection level for mice that received BDNF (57.1 nmol/kg)+ADTC5 (10 μmol/kg; A1, A2), BDNF (28.6 nmol/kg)+ADTC5 (10 μmol/kg; A3), or BDNF alone (28.6 nmol/kg; B1, B2, B3). FIG. 9G provides a graphical representation of total protein loaded among all groups. Contrast and brightness of images were adjusted only for display purposes.

### Representative Examples Illustrating Non-Invasive Brain Delivery and Efficacy of BDNF Provided By Compounds and Methods of the Present Technology in APP/PS1 Transgenic Mice as an Alzheimer's Disease Model

Animals: All animal studies were carried out under the approved animal protocol (AUS-74-11) granted by Institutional Animal Care and Use Committee (IACUC) at The University of Kansas. Animal Care Unit (ACU) personnel and veterinarians were involved in the care of the animals used in this study. Female transgenic APP/PS1 (MMRRC stock #34832-Jax) were obtained from Jackson Laboratory (Bar Harbor, Me.) and housed until at least 6 months of age. Mice received intravenous (i.v.) injections of either BDNF (5.7 nmol/kg)+ADTC5 (10 μmol/kg; n=7), BDNF alone (5.7 nmol/kg; n=6), or vehicle (n=6) every 4 days, for a total of 8 injections. At the end of the study, the mice were euthanized via CO₂ inhalation and perfused with PBS immediately followed by 4% formalin fixative solution. The brains were extracted and post-fixed overnight in the perfusion-fixation solution then transferred to 70% ethanol PBS solution for paraffin embedding.

Peptide synthesis and purification: ADTC5 peptide was synthesized using a solid-phase peptide synthesizer (Gyros Protein Technologies, Tucson, Ariz.) as described above in this disclosure. Briefly, crude peptide was cleaved from the resin with TFA containing scavengers followed by precipitation in cold diethyl ether. The disulfide bond in ADTC5 was formed by stirring the linear peptide precursor in 0.1 M ammonium bicarbonate buffer solution at pH 9.0 in high dilution while bubbling air through the solution. The cyclic ADTC5 was purified using a semi-preparative HPLC X-bridge C18 column (Waters, Milford, Mass.) and the product was analyzed by analytical HPLC to be >95% pure. The exact mass of cyclic ADTC5 was determined by mass spectrometry.

Y-maze assessment: Twenty-four hours following the 8^{th} injection of the treatment, the mice were subject to Y-maze behavioral assessment. See Kim, H. Y., Kim, H. V., Yoon, J. H., Kang, B. R., Cho, S. M., Lee, S., Kim, J. Y., Kim, J. W., Cho, Y., Woo, J.,Kim, Y., Taurine in drinking water recovers learning and memory in the adult APP/PS1 mouse model of Alzheimer's disease. Sci Rep 2014; 4:7467; Webster, S. J., Bachstetter, A. D., Nelson, P. T., Schmitt, F. A.,Van Eldik, L. J., Using mice to model Alzheimer's dementia: an overview of the clinical disease and the preclinical behavioral changes in 10 mouse models. Front Genet 2014; 5:88. First the mice were habituated to the maze for 8 min with one arm of the maze closed off. Three hours following habituation, the mice were re-introduced to the maze for 5 min with all three arms open. All mice were initially placed in the center of the maze oriented toward the same arm; the maze was thoroughly cleaned with 70% ethanol and Virkon between each trial to remove scent cues. Time in Novel Arm was defined as the percent of total time (5 min) spent in the third arm of the maze (previously closed-off arm). An entry into an arm was defined as the head of the mouse entering.

Novel object recognition assessment: Twenty-four hours following the Y-maze assessment, the mice were subjected to Novel Object Recognition (NOR) assessment. See Webster, S. J., Bachstetter, A. D., Nelson, P. T., Schmitt, F. A.,Van Eldik, L. J., Using mice to model Alzheimer's dementia: an overview of the clinical disease and the preclinical behavioral changes in 10 mouse models. Front Genet 2014; 5:88. First, mice were individually habituated in an empty open field for 5 min. Twenty-four hours after habituation, 2 identical objects were placed in the open field, 5 cm away from the wall; there were two different sets of identical objects that were randomly selected for each mouse. Mice were individually placed in the field facing away from the objects and were allowed to familiarize themselves with the objects for 10 min. Twenty-four hours after familiarization phase, mice were re-subjected to the open field, but one of the objects was replaced with a novel object. The position of the novel object (right or left side) was randomized for each mouse. The mice were allowed to explore the objects for 10 min and the total amount of time each mouse spent interacting with each object was measured. For all steps, the open field and object were cleaned with 70% ethanol and Virkon.

### Histology and Immunohistochemistry

Coronal brain sections (10 μm thickness) were generated and mounted onto gelatin-coated slides (Superfrost Plus, Thermo Fisher Scientific, Waltham, Mass.). For both Aβ histology and NG2 receptor immunohistochemistry, sections were deparaffinized using xylene and serially hydrated from 95% ethanol to distilled water. The positive and negative controls were carried out according to Hewitt, S. M., Baskin, D. G., Frevert, C. W., Stahl, W. L., Rosa-Molinar, E., Controls for immunohistochemistry: the Histochemical Society's standards of practice for validation of immunohistochemical assays. J Histochem Cytochem 2014; 62:693-7. For Aβ, slides were stained with Congo Red Solution (Abcam, Cambridge, UK) for 20 min, then dipped in twice in 100% ethanol, cleared with xylene, mounted using synthetic Permount (Fisher Scientific, Hampton, N.H.) and covered using 2.5 coverslips. Aβ plaque levels were quantified by counting the number of plaques from the hippocampus at 10× magnification from 5 random sections per group (n=5).

For anti-NG2 mAb staining, slides were first blocked in a 3% hydrogen peroxide blocking agent then subsequently rinsed using distilled water. Next, heat-induced isotope retrieval (HIER) was performed using a 10 nM sodium citrate buffer at pH 6.0. Briefly, the HIER buffer was brought to a boil and slides were submerged for 15 min in HIER followed by immediate rinsing with PBS containing 0.05% Tween-20 (PBS-T) buffer for 3 min. Slides were then blocked using 10% normal bovine serum albumin (BSA) for 6 min and subsequently rinsed with water. The NG2 primary antibody (Abcam, Cambridge, UK) was then applied to the slides in a dilution of 1:1,000 in PBS-T followed incubation overnight at 4° C. in a moisturizer chamber. The following steps were performed using the Polink-2 HRP plus rabbit DAB detection system for immunohistochemistry (Golden Bridge International Labs, Bothell, Wash.). Briefly, rabbit antibody enhancer (Reagent 1) was applied to the slides and incubated at room temperature for 30 min. The slides were then rinsed with PBS-T and Polymer-HRP for rabbit (Reagent 2) was applied followed by incubation at room temperature for 30 min. The slides were then rinsed with PBS-T and the chromogen was applied. To prepare the chromogen, 2 drops of DAB Chromogen (Reagent 3B) were added to DAB Reagent buffer (Reagent 3A). The slides were incubated with the DAB mixture for 10 min and then were rinsed with water. Lastly, the slides were dipped into 100% ethanol twice, dried, mounted using Permount and 1.5 coverslips. Anti-NG2-stained slides were imaged using a Leica DM750 Compound Bright-Field Upright Microscope and imaged at 40× (0.65 NA; HI PLAN ∞) magnification under identical exposure times. Anti-NG2 mAb levels were quantified via densitometry analysis at 40× magnification from 5 random sections per group (n=5).

### Fluorescent in situ Hybridization

Coronal brain sections (10 μm thickness) were washed three times in PBS before mounting on gelatin-coated glass slides (Superfrost Plus, Thermo Fisher Scientific). Tissues were allowed to dry at RT and were then stored at −20° C. until use. Fluorescence in situ hybridization (FISH) was performed using Multiplex Reagent Kit V2 from RNAscope® Technology 2.0 (Advanced Cell Diagnostics (ACD), Hayward, Calif.). See Vasquez, J. J., Hussien, R., Aguilar-Rodriguez, B., Junger, H., Dobi, D., Henrich, T. J., Thanh, C., Gibson, E., Hogan, L. E., McCune, J., Hunt, P. W., Stoddart, C. A.,Laszik, Z. G., Elucidating the Burden of HIV in Tissues Using Multiplexed Immunofluorescence and In Situ Hybridization: Methods for the Single-Cell Phenotypic Characterization of Cells Harboring HIV In Situ. J Histochem Cytochem 2018; 66:427-46; Gershon, T. R., Crowther, A. J., Liu, H., Miller, C. R.,Deshmukh, M., Cerebellar granule neuron progenitors are the source of Hk2 in the postnatal cerebellum. Cancer Metab 2013; 1:15; and Smith, P. A., Schmid, C., Zurbruegg, S., Jivkov, M., Doelemeyer, A., Theil, D., Dubost, V., Beckmann, N., Fingolimod inhibits brain atrophy and promotes brain-derived neurotrophic factor in an animal model of multiple sclerosis. J Neuroimmunol 2018; 318:103-13. Mounted tissue sections were deparaffinized using xylene and serially dehydrated in 50%, 70%, 95%, and 100% ethanol for 5 min each. Between all pretreatment steps, tissue sections were briefly washed with distilled water. Pretreatment solution 1 (hydrogen peroxide reagent) was applied for 10 min at RT, and then the tissue sections were boiled in pretreatment solution 2 (target retrieval reagent) for 15 min. Mounted slices were pretreated with solution 3 (protease reagent) for 30 min at 40° C. in the HybEz™ hybridization system (ACD). Following tissue pretreatment, the following transcript probes were applied to all sections: Mm-Mapk1-C1 (Cat. #458161), Mm-Arc-C2 (Cat. #316911-C2), and Mm-Egr1-C3 (Cat. #423371-C3), which correspond to MAPK1, ARC, and EGR1, respectively. Probes were hybridized into the brain sections for 2 h at 40° C. and subsequently washed for 2 min at room temperature. Following hybridization, hybridize AMP 1 was applied to each slide, which was then incubated for 30 min at 40° C. The same process was repeated for hybridize AMP 2 and 3. For HRP-C1 signal development (MAPK1), HRP-C1 was applied to the slides, and they were incubated for 15 min at 40° C. and then washed. For C1, Opal® 650 (Akoya Biosciences, Menlo Park, Calif.) was applied and incubated for 30 min at 40° C. and then washed. Following the wash, HRP blocker was applied to each slide, incubated for 15 min at 40° C. followed by washing. This process was repeated for C2 (ARC), and C3 (EGR1) using Opal® 620 and 520, respectively. The resulting transcript-fluorophore labeling is as follows: MAPK-650, ARC-620, EGR1-520. All sections were counterstained by incubating DAPI (4′,6-diamidino-2-phenylindole), fluorescent DNA stain for 30 sec at room temperature following by rinsing. Slides were then covered using ProLong Gold Antifade Mounting Media and 1.5 coverslips. Slides were allowed to dry in the dark overnight at 4° C. All sections were imaged within 2 weeks.

Fluorescent images were taken using an Olympus IX-81 inverted epifluorescence microscope XI81 (Olympus Life Solutions, Waltham, Mass.) running SlideBook Version 6.0 (3i, Ringsby, Conn.) equipped with a digital CMOS camera (2000×2000), automatic XYZ stage position, ZDC autofocus, and a xenon lamp excitation source. Images were taken under identical exposure times (100 msec) using a 40× objective (0.95 NA; UPlanSApo ∞) and appropriate filter sets for each stain or Opal® fluorophore (i.e., DAPI-DAPI, FITC-Opal® 520, Texas red-Opal® 620, and Cy 5.5-Opal® 650). To determine the degree of mRNA transcript expression, 4 images of the CA1 region of the hippocampus regions were randomly selected from mouse samples of each group, and the total fluorescence signal intensity for each channel was quantified. For display purposes, images were pseudo-colored and brightness-adjusted using ImageJ; green was assigned to Opal® 520 (EGR1), red to Opal® 620 (ARC), cyan to Opal® 650 (MAPK1), and grey to DAPI.

Statistics and Data Analysis: All statistics and data analyses were performed using GraphPad Prism (San Diego, Calif.). Analysis of variance (ANOVA) and Student's T-test were performed with ap-value of less than 0.05 used as the criterion for statistical significance unless otherwise stated.

### Results

### Effect of BDNF on Cognitive Performance in Y-maze and NOR Assessments

The ability of ADTC5 to deliver BDNF into the brains of mice after i.v. injection was assessed by determining the effects of BDNF on improving cognitive function in APP/PS1 Alzheimer's disease animal model as determined by Y-maze and NOR assessments. The efficacy of BDNF (5.71 nmol/kg)+ADTC5 (10 μmol/kg; n=7) was compared to that of BDNF alone (5.71 nmol/kg; n=6), and vehicle (n=6). Once mice reached 6 months of age, each treatment was delivered via an i.v. injection every 4 days for a total of 8 injections. Twenty-four hours following the final injection, mice were subjected to Y-maze and NOR assessments.

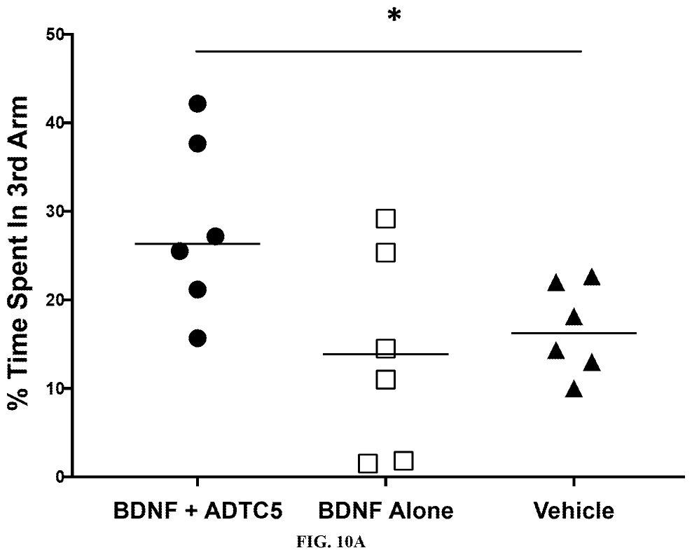

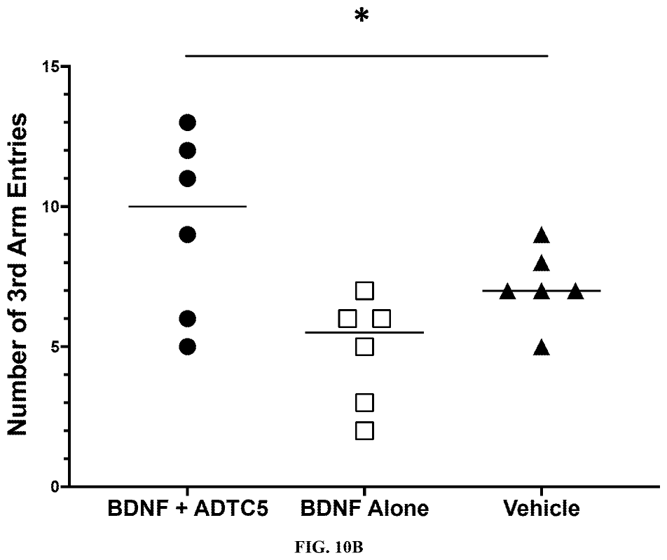

In the Y-maze, mice that received BDNF+ADTC5 performed significantly better than mice that received BDNF alone or vehicle (FIGS. 10A-10B). The mice that received BDNF+ADTC5 spent a greater percentage of time in the third arm (F_{(2,15)}=3.99; p<0.05, FIG. 10A) and had a higher number of entries into the third arm of the maze than did the groups that received BDNF alone or vehicle (F_{(2,15)}=5.63; p<0.05, FIG. 10B).

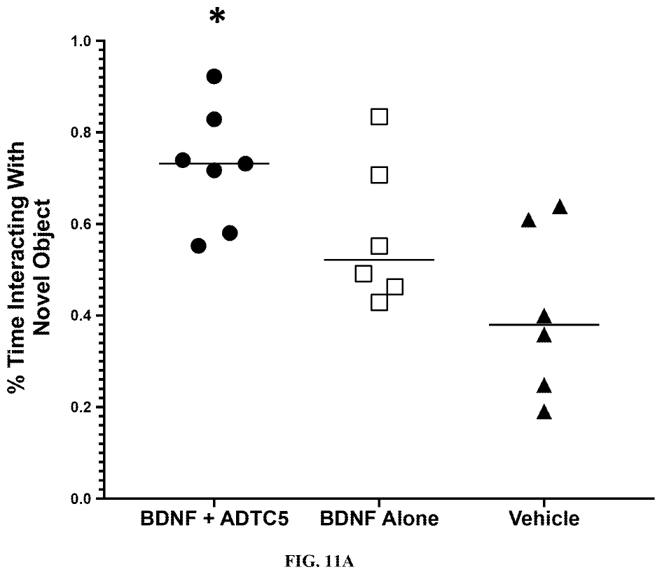

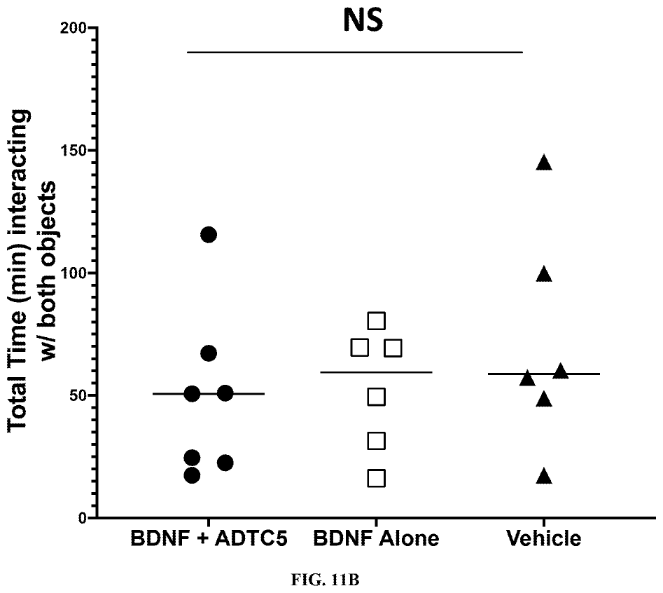

For the NOR assessment, the mice that received BDNF+ADTC5 performed significantly better than mice that received BDNF alone or vehicle. The mice that received BDNF+ADTC5 spent a greater percentage of time with the novel object (F_{(2,16)}=6.55; p<0.01) than did the mice that received BDNF alone or vehicle (FIG. 11A). Lastly, there was no significant difference in total time spent with either of the two objects; in other words, all groups spent similar amounts of time interacting with either object (F_{(2,16)}=0.682; p=0.52; FIG. 11B).

It is expected that performing similar studies as described herein with HAVN1, HAVN2, ADTN1, ADTN2, and/or cyclic ADTHAV will provide results that are similar or significantly improved.

### Effect of BDNF Delivery on Amyloid Beta Plaques in Hippocampus

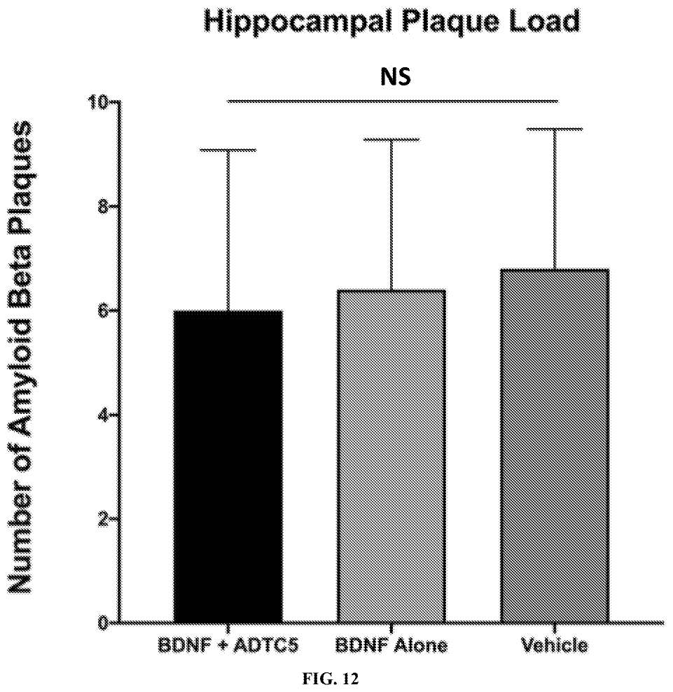

The effects of BDNF brain delivery on the amounts of Aβ plaques were determined in groups of mice treated with BDNF+ADTC5, BDNF alone, or vehicle. The results indicated that all groups expressed high level and variability of plaques in the hippocampus regardless of the treatment. There were no significant differences in the amounts of amyloid beta plaques in all three groups (F_{(2,12)}=0.096; p=0.91, n=5; FIG. 12).

### Effect of BDNF Delivery on NG2-Glia

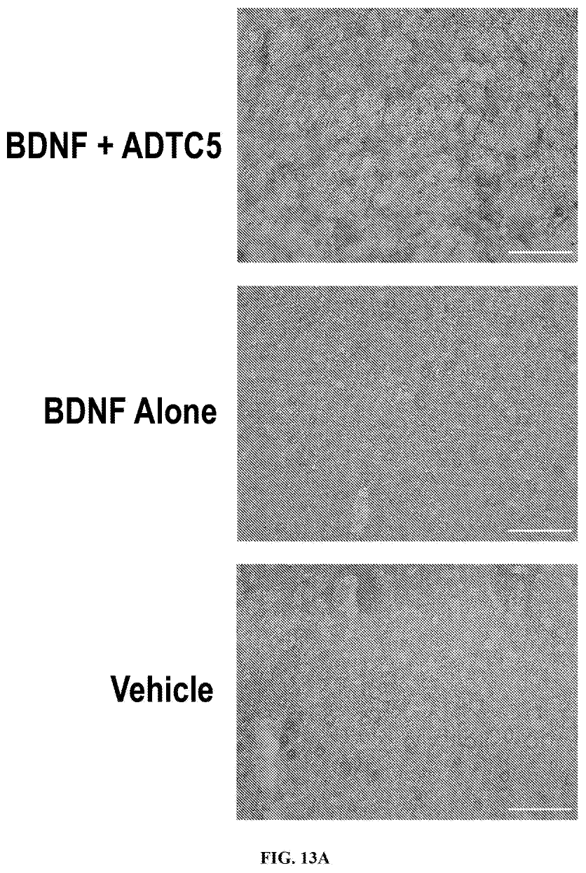

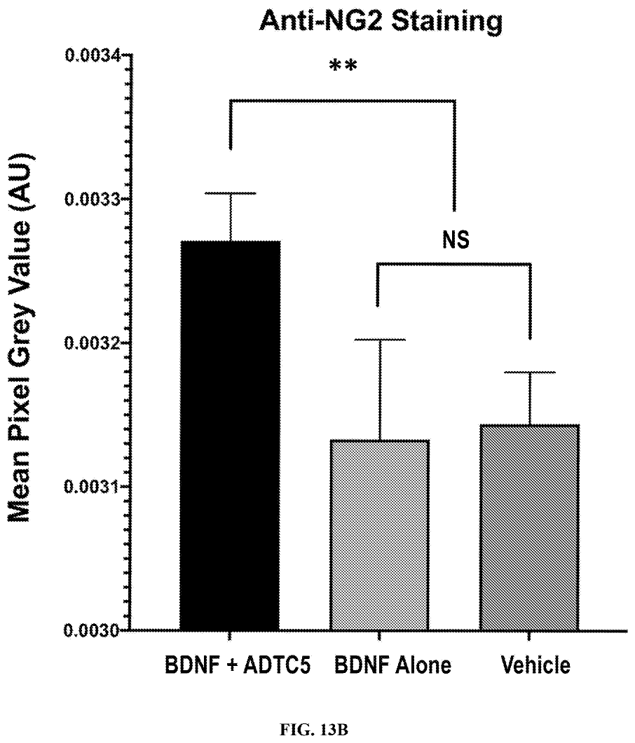

As shown above in this disclosure, brain delivery of BDNF using ADTC5 in experimental autoimmune encephalomyelitis (EAE) induced oligodendrocyte maturation which was reflected in the increase in NG2 receptor expression in the brain. Furthermore, BDNF^{+/+} mice have shown a significant upregulation of NG2 glia following the development of cuprizone-induced lesions compared to BDNF^{+/-} and BDNF^{-/-} mice. Thus, the effects of BDNF brain delivery using ADTC5 were determined by evaluating the oligodendrocyte progenitor maturation in the APP/PS1 mouse model. In this case, the brain expressions of NG2 receptors were probed in the cortex region using anti-NG2 antibody staining. The brain cortexes of mice treated with BDNF+ADTC5 have higher degree of NG2 staining (darker staining) compared to those treated with BDNF alone or vehicle alone (FIG. 13A). Quantification using pixel values of NG2 stain indicated that mice were treated with BDNF+ADTC5 had a higher or darker staining compared to those mice treated with BDNF alone or vehicle (FIG. 13B, F_{(2,12)}=11.16; p<0.01, n=5). It is expected that performing similar studies as described herein with HAVN1, HAVN2, ADTN1, ADTN2, and/or cyclic ADTHAV will provide results that are similar or significantly improved.

### Effect of BDNF on EGR1, ARC, and MAPK1 mRNA Transcript Expression

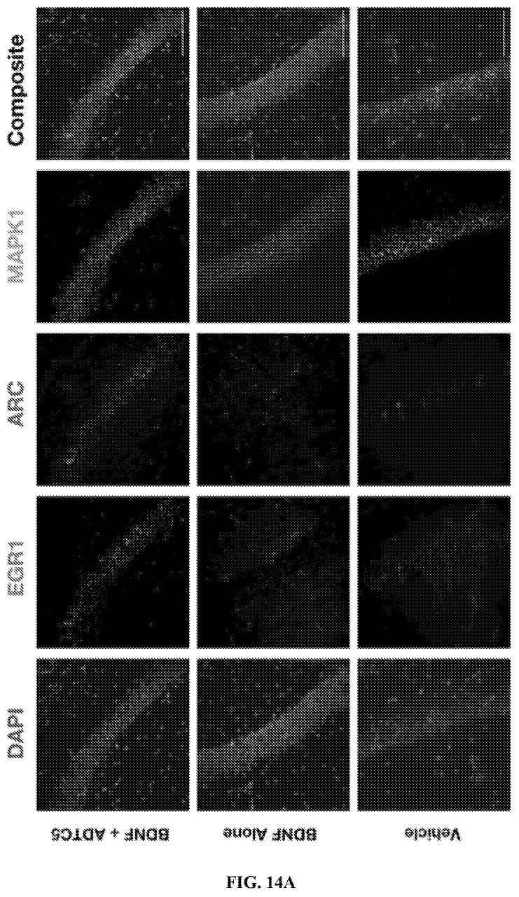

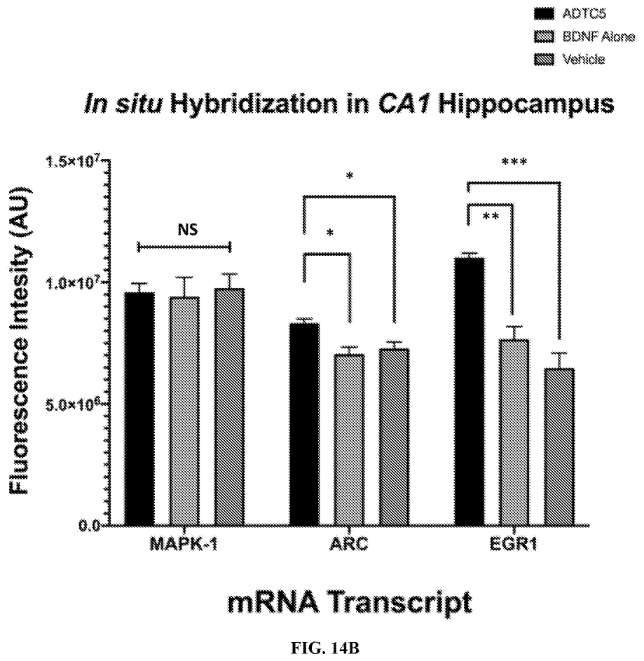

BDNF is known to stimulate downstream transcription factors such as, tropomyosin receptor kinase B (TrkB), cyclic AMP response element binding protein (CREB), MAPK1, EGR1, and ARC. We have previously demonstrated that delivering BDNF using ADTC5 to EAE mice resulted in the increase in EGR1 and ARC mRNA transcript expression. Additionally, others have shown that EGR1 directly targets ARC expression. The levels of EGR1, ARC, and MAPK1 mRNA transcripts were quantified via fluorescence in situ hybridization (FISH) method. Qualitatively, the brain sections from the CA1 regions of the hippocampus have higher levels of visual staining from ERG1 and ARC expression in mice treated with BDNF+ADTC5 compared to mice treated with BDNF alone or vehicle (FIG. 14A). However, it was difficult to visually differentiate the staining of MAPK1 in all three different groups. Using quantitative method, the number of pixel counts from EGR1 (F_{(2,9)}=23.48; p<0.001, n=5) and ARC (F_{(2,9)}=7.33; p<0.05, n=5) transcript levels in BDNF+ADTC5 group were significantly higher compared to those mice received BDNF alone or vehicle (FIG. 14B). In contrast, very high levels of MAPK1 expression were found in all three groups with no significant differences were observed in all groups (F_{(2,9)}=0.08; p=0.92, n=5; FIG. 14B). It is expected that performing similar studies as described herein with HAVN1, HAVN2, ADTN1, ADTN2, and/or cyclic ADTHAV will provide results that are similar or significantly improved.

### Representative Examples Illustrating In Vivo Brain Delivery and Brain Deposition of Proteins of Various Sizes Via Compounds and Methods of the Present Technology

### Materials and Methods

Chemicals, Reagents, and Animals. Gyros Protein Technologies, Inc. (Tucson, Ariz.) was used as a vendor to purchase amino acids and coupling reagents for the automated peptide synthesizer. IRdye-800CW-NHS ester and IRdye-800CW Donkey anti-Goat IgG were purchased from LI-COR, Inc. (Lincoln, Nebr.). Sigma Aldrich Chemical Company (St. Louis, Mo.) and Fisher Scientific, Inc. (Hampton, N.H.) were used as suppliers or proteins and reagents in this study. Protocols used for all animal studies have been approved by the Institutional Animal Care and Use Committee (IACUC) at The University of Kansas. All animals were cared by the Animal Care Unit personnel at The University of Kansas under the supervision of Veterinarians.

Peptide Synthesis and Purification. The synthesis of the linear or cyclic peptides was accomplished using a Tribute solid-phase peptide synthesizer from Gyros Protein Technologies, Inc. (Tucson, Ariz.), as discussed previously in this disclosure. A TFA solution containing scavengers was used to cleavage the peptide from the resin followed by addition of the TFA solution into cold diethyl ether to precipitate the peptide. To form cyclic ADTC5 peptide with a disulfide bond, the linear precursor was dissolved in high dilution using bicarbonate buffer solution at pH 9.0 followed by bubbling air into the peptide solution. The resulting oxidation reaction contained a high yield of the desired cyclic monomer with minimal amounts of side products (e.g., dimers and oligomers). The monomer was isolated from the mixture using a semi-preparative C18 column Waters XBridge C18 (19 mm×250 mm, 5 μm particle size; Waters Corporation, Milford, Mass.)) in HPLC. The purity of each isolated fraction was determined by analytical HPLC using C18 column (Luna C18 (4.6 mm×250 mm, 5 μm particle size, 100 Å; Phenomenex, Inc., Torrance, Calif.)). The identity of each peptide was confirmed by mass spectrometry.

### Conjugation of Proteins with IRdye-800CW-NHS Ester

Lysozyme, albumin, and fibronectin used in this study were conjugated with IRdye-800CW according to the manufacturer's instructions. Briefly, dyes were reacted with 1 mg/mL of protein in PBS with 10% potassium phosphate buffer, pH 9 (v/v) for 2 h at 25° C. The resulting conjugates were purified using a spin column called Zeba Spin Desalting Column with 7 kDa molecular weight cut-off (Fisher Scientific, Inc. (Hampton, N.H.)). The purity of each conjugate was determined using SDS-PAGE, and the conjugate band was scanned (Excitation=778 nm; Emission=794 nm) with an Odyssey CLx NIR scanner to ensure that there was no free IRdye-800CW in the protein conjugate solution. Once any free dye was removed, the degree of labeling was determined using a UV spectrophotometer (Varian Cary 100, Agilent) to measure the fluorophore absorption and the protein absorbance at 280 nm, corrected for the fluorophore.

The protein concentration is calculated using the formula,

$$Protein Conc . ( \frac{mg}{mL} ) = \frac{{A}_{280} - ( 0.03 \times {A}_{780} )}{{ɛ}_{Protein}} \times {MW}_{Protein} \times Dilution Factor$$

in which 0.03 was utilized as a correction factor for IRDye-800CW absorbance; the absorbance at 280 nm equals to 3.0% of the absorbance at 780 nm. εₚᵣₒₜₑᵢₙ was designated as the molar extinction coefficient of the protein and molecular weight of the protein was designated as MW_{Protein}.

### NIRF Method to Quantify Protein Amount in the Brain

Preparation of Stock and Standard Curves. The stock solution for IRDye800CW protein (i.e., lysozyme, 70 μg/mL) was prepared and stored at −80° C. The stock solution was later diluted with PBS to make the required standard solutions. To produce a standard calibration curve, 200 μL of blank brain homogenates was spiked with 10 μL standard solutions of various concentrations to yield a linear range from 0.5 to 50 ng/mL. The same method was employed for the sample's quality control (QC).

Accuracy and Precision. For precision studies, IRDye-800CW-lysozyme was used. Brain homogenates were spiked with protein at concentrations between 0.5 and 50 ng/mL for determining the intra-day and inter-day, accuracy and precision.

Evaluation of Method Stability. To evaluate stability of the quantitative method, IRDye-800CW-labeled lysozyme was used in spiked brain homogenates under various temperature and storage conditions. Three sets of samples were prepared to evaluate these various conditions. First, the samples were incubated at room temperature for 6 h before analysis. Second, the samples were incubated at −20° C. for 24 h with subsequent unassisted thawing at room temperature. Third, the samples were subjected to three freeze-thaw cycles between −20° C. and room temperature over a 24-h period prior to analysis. These stability studies were accomplished using protein concentrations from 0.5 to 50 ng/mL and three repeats for each sample group.

Brain Delivery IRdye800CW-labeled IgG mAb using ADTC5 in SJL/elite Mice. For initial evaluation of whether ADTC5 can deliver proteins into the brain, IRdye800CW donkey-anti-goat IgG mAb was administered via i.v. with and without ADTC5 peptide in 5-8-week-old SJL/elite mice. Two groups of healthy SJL/elite mice were injected with (a) a mixture of IgG mAb (26.8 nmol/kg) and ADTC5 peptide (13 μmol/kg) (n=5) and (b) IgG mAb alone (26.8 nmol/kg) (n=4). After 15 min in the systemic circulation, the mice were euthanized using CO₂ inhalation followed by brain perfusion using PBS to remove the remaining protein in the BBB microvasculature. Next, the brains were isolated followed by NIRF imaging using Licor Odyssey CLx (Licor, Lincoln, Nebr.). Eight optical sections were taken at 0.5 mm increments beginning from the bottom surface of the brain to a depth of 4 mm. The optical sections were summed to yield a fluorescence intensity value per each brain.

### Comparison of HAV6 and ADTC5 in Delivering Various Sizes of Proteins Into C57BL/6 Mice

The BBB modulatory activities of ADTC5 and HAV6 to enhance brain delivery of IRdyeR800CW-labeled lysozyme, albumin, IgG mAb, and fibronectin were compared in C57BL/6 mice. The proteins with or without 13 μmol/kg HAV6 or ADTC5 were administered via tail vein injection. For lysozyme, the delivered doses were 21.6 and 54 nmol/kg. For albumin, IgG mAb, and fibronectin, the dose used was 21.6 nmol/kg. Fifteen minutes after IgG mAb administration with or without a peptide of the present technology, the animals were sacrificed and PBS with 0.5% Tween-20 was administered for cardiac perfusion to remove the remaining protein in the BBB microvessels. The brain and other organs such as lung, heart, spleen, liver and kidney were harvested and rinsed with PBS. Protein depositions in the brain and other organs were quantified by NIRF imaging using an Odyssey CLx NIRF scanner.

A second quantification method was done using brain homogenates. In this case, the brains were homogenized in 2.0 mL PBS by mechanical disruption and 200 μL of homogenized brain (n=8) was aliquoted to a 96-well plate followed by quantification using the Odyssey CLx scanner. The signal intensity was compared to calibration curve and normalized to brain weight and homogenate volume.

Brain Perfusion. After euthanasia using CO₂ chamber, mice were immediately subjected to cervical dislocation followed by removal of IRdye800CW-labeled protein from the brain capillaries using perfusion solution. In this case, a solution containing PBS with 0.2% Tween-20 was transcardially perfused to remove the remaining protein molecules in the brain endothelial microvessels. After perfusion, the brain was removed from the skull and subjected to capillary depletion.

Capillary Depletion Method. Parallel capillary depletion experiments were performed as described by Triguero, D.; Buciak, J.; Pardridge, W. M. Capillary depletion method for quantification of blood-brain barrier transport of circulating peptides and plasma proteins. Journal of neurochemistry 1990, 54, (6), 1882-8 to ensure that there was no trapping of the delivered molecules in the BBB microvessel endothelial cells. IRdye800CW-labeled protein was added and mixed into brain homogenates; then, the mixture was divided into two sets. A 500 μL set of homogenates was mixed with 500 μL of PBS while another set of 500 μL homogenates was mixed with 500 μL of 26% dextran solution. Both sets were centrifuged at 5,400 g for 15 min at 4° C. and 200 μL of supernatant was collected for analysis using the Odyssey CLx scanner.

Statistical Analysis. The data from the brain delivery of various sized molecules were analyzed and compared using ANOVA with Student-Newman-Keuls for indication of statistical significance. The criterion for statistical significance was selected for the p-value of less than 0.05.

### Results

Synthesis and Purification of IRdye800CW-labeled Proteins. To make IRdye800CW-labeled lysozyme, albumin, or fibronectin, IRdye800CW-NHS was reacted to free amino groups of the respective protein to form stable conjugates. To purify the protein conjugates, the excess of IRdye800CW-NHS was removed from the reaction mixture using a Pierce Zeba desalting spin column with a cut-off molecular weight of 7 kDa. The purified conjugates were evaluated with SDS-PAGE scanned with an Odyssey CLx NIR imager. Lysozyme and albumin conjugates showed a single band while fibronectin had a faint lower fragment band; all proteins have the appropriate mass without unreacted IRdye. The final protein concentrations for lysozyme, albumin, and fibronectin were determined to be 1.35, 1.68, 2.30 mg/mL, respectively.

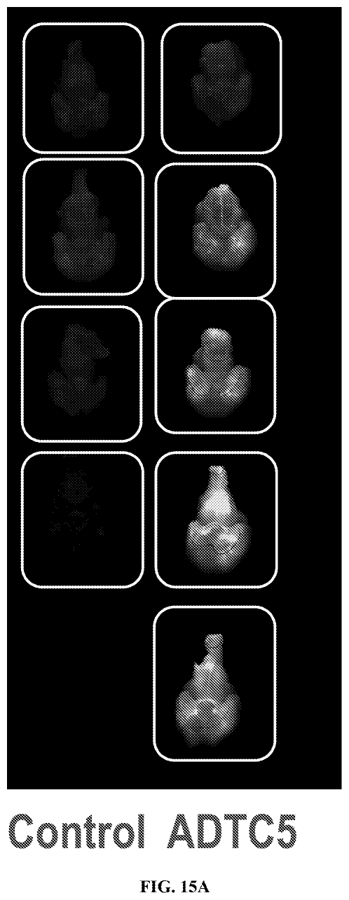

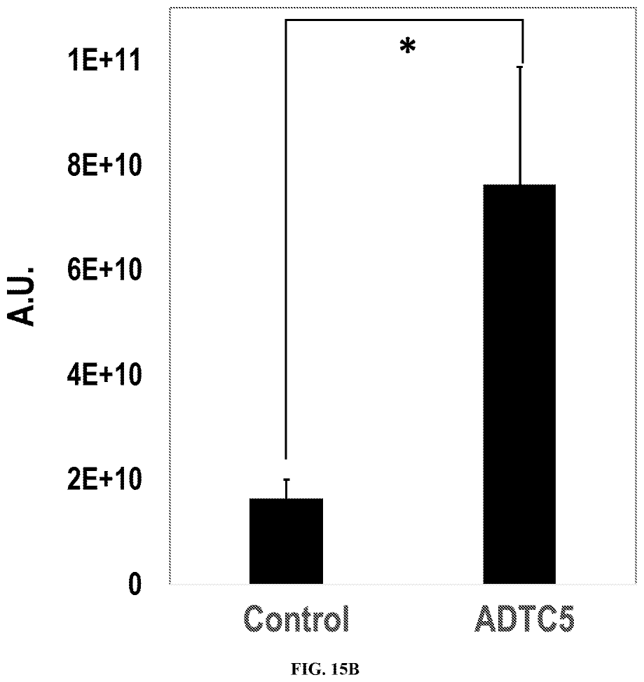

Initial Brain Delivery of IRDye800CW-IgG mAb by ADTC5 in SJL/Elite Mice. In this study, IgG mAb was administered via i.v. in SJL/elite mice in the absence or presence of ADTC5 peptide. Prior to injection, IgG mAb identity was evaluated using SDS-PAGE gel and showed a major band at ˜150 kDa with very light bands for ˜100 kDa heavy and ˜50 kDa light chains (data not shown). There was no observation of the band for IRDye800CW alone. To remove the excess IgG mAb from the brain capillaries, the mice were perfused with PBS+Tween-20 perfusion solution. After brain extraction, the brain scans of mice treated with IgG mAb alone showed very low NIRF image in eight different levels of brain scans (n=4) (FIG. 15A). In contrast, the mouse brains administered with IgG mAb and ADTC5 showed strong NIRF signals on eight different brain scan levels (n=5) (FIG. 15A). Quantitative accumulation of NIRF signals from all scan levels indicated that the brains from mice treated with IgG mAb+ADTC5 had a significantly higher signal intensity than those of mice treated with IgG mAb alone (FIG. 15B). In summary, ADTC5 increases the brain delivery of IgG mAb in C57BL/6 mice.

### Method Development and Validation of NIRF Quantification

Linearity, Accuracy, and Precision. The lowest limit of detection (LLOD) and intra-day as well as inter-day precision and accuracy were determined using a calibration curve generated with concentrations from 0.5 to 50 ng/mL. The calibration curve was generated by plotting concentrations of standard vs. NIRF intensity from the Odyssey CLx imaging system. The resulting standard curve has good linearity with R²≥0.98 and LLOD of 0.3 ng/mL. Three different protein concentrations were used to determine intra-day and inter-day accuracy and precision and for obtaining % RSD and % RE (Table 1). The acceptable analytical method was determined when the % RSD and % RE values were less than 15%.

TABLE 1
Precision and Accuracy

| Concentration | Intra-day | | Inter-day | |
|---|---|---|---|---|
| (ng/mL) | % RSD | % RE | % RSD | % RE |
| 0.5 | 15.1 | 7.1 | 10.5 | 6.4 |
| 5.0 | 4.6 | −2.8 | 3.4 | 5.8 |
| 50.0 | 2.8 | 5.4 | 3.8 | 1.6 |

Stability Assay. The stability of the analyte during evaluation was investigated using IRDye800CW-lysozyme in three concentrations at two temperatures and a freeze-thaw condition (Table 2); in different analyte concentrations, the % RSD was less than 15% at room temperature for 6 h. Thus, this condition was used in this study. In contrasts, the two other conditions were determined to be unacceptable for this study because the % RSD was higher than 15%.

TABLE 2
Stability of Protein

| | % RSD | | |
|---|---|---|---|
| | Room | | |
| Concentration | Temp. | −20° C. | Three freeze- |
| (ng/mL) | (6 h) | (24 h) | thaw cycles |
| 0.5 | 7.7 | 16.2 | 23.1 |
| 5.0 | 5.4 | 18.5 | 8.5 |
| 50.0 | 3.7 | 25.1 | 18.4 |

Comparison of HAV6 and ADTC5 in Enhancing Brain Delivery of Various Proteins. In this study, the activities of HAV6 and ADTC5 peptides to deliver various sized proteins (i.e., lysozyme, albumin, IgG mAb, and fibronectin) in C57BL/6 mice were compared in a quantitative manner. The resulting calibration curve generated from 0.5 to 50 ng/mL of lysozyme produced a linear curve with R²≥0.99. Similar calibration curves were generated for albumin and IgG mAb. The amount of protein in the brain (Table 3) was determined by interpolation of NIRF intensity of the brain homogenate into the standard curve.

TABLE 3
Quantitative Amounts of Proteins in the Brain

| Protein | Group | pmol/g brain |
|---|---|---|
| Lysozyme | Control | 0 ± 0 |
| | HAV6 | 8.3 ± 2.5 |
| | ADTC5 | 37.8 ± 7.1 |
| Albumin | Control | 11.8 ± 1.0 |
| | HAV6 | 15.5 ± 3.1 |
| | ADTC5 | 40.7 ± 7.4 |
| IgG mAb | Control | 4.0 ± 0.4 |
| | HAV6 | 3.4 ± 0.5 |
| | ADTC5 | 13.3 ± 0.7 |

### Brain Delivery of 15 kDa Lysozyme and Peripheral Organ Distributions

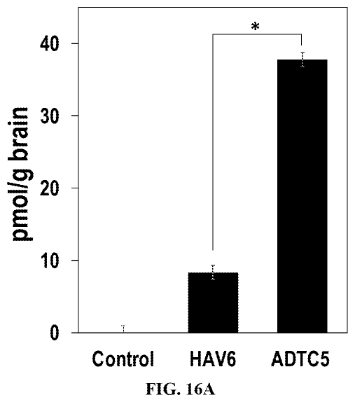

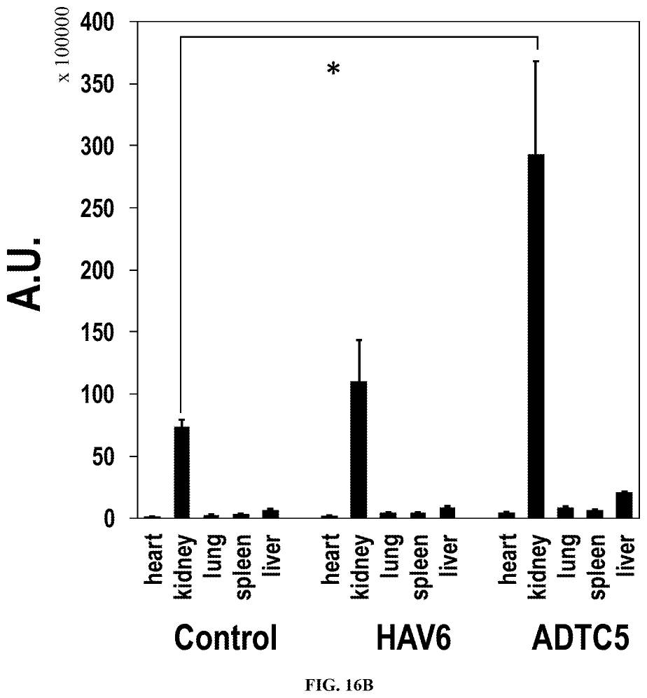

The first delivery of lysozyme was carried out at a dose of 21.6 nmol/kg with 13 μmol/kg of HAV6 or ADTC5 peptide, and no significant improvement was observed in the brain compared to lysozyme alone (data not shown). Next, the dose of lysozyme was increased to 54 nmol/kg with 13 μmol/kg of HAV6 or ADTC5 peptide (FIGS. 16A-16B). Prior to brain extraction for NIRF imaging, the mice were perfused to remove the remaining lysozyme in the brain capillaries. Through visual observation, the NIRF brain images of mice treated with HAV6 +lysozyme and ADTC5+lysozyme appeared to show higher intensity than those treated with lysozyme alone. The NIRF intensity of the ADTC5 group was higher than that of HAV6 group. Quantitatively, the average amount of lysozyme in the ADTC5 group (37.8±7.1 pmol/g brain) was significantly higher than that in the HAV6 group (8.3±2.5 pmol/g brain, p <0.05) (FIG. 16A, Table 3). The lysozyme amounts in the brains of both peptide groups were higher than that of control group, which was below the detection limits. The results suggest that ADTC5 is a better BBB modulator than HAV6. To ensure that the brain perfusion procedure eliminated any residual molecule in the BBB microvessels, the brain capillary depletion was carried out using the brain homogenates. The capillary depleted samples were compared to non-depleted samples. The difference between the capillary depleted and non-depleted samples was less than 1.9%, indicating the that the perfusion method was satisfactory in removing almost all the labeled protein from the brain capillaries.

The effects of HAV6 and ADTC5 in lysozyme distributions in kidney, lung, heart, spleen, and liver were also determined. Visually, the most intense NIRF images were in the kidney in all three groups, with the highest image intensity on ADTC5 group. Quantitative data confirmed that lysozyme deposition in the kidney was the highest in the ADTC5-treated group, followed by the HAV6-treated group and control (FIG. 16B). It is not surprising that the lysozyme undergoes glomerular filtration in the kidney because of its molecular weight being lower than 65 kDa.

### Brain Delivery of 65 kDa Albumin and Peripheral Organ Distributions

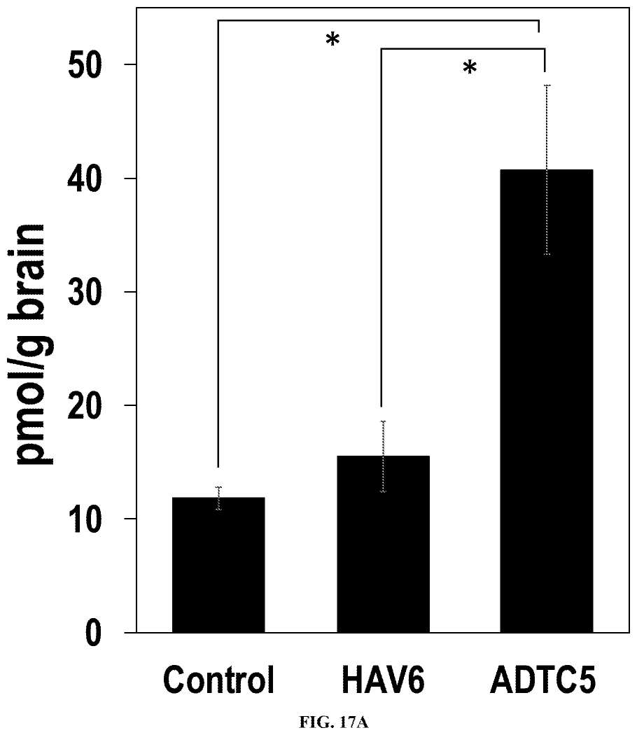

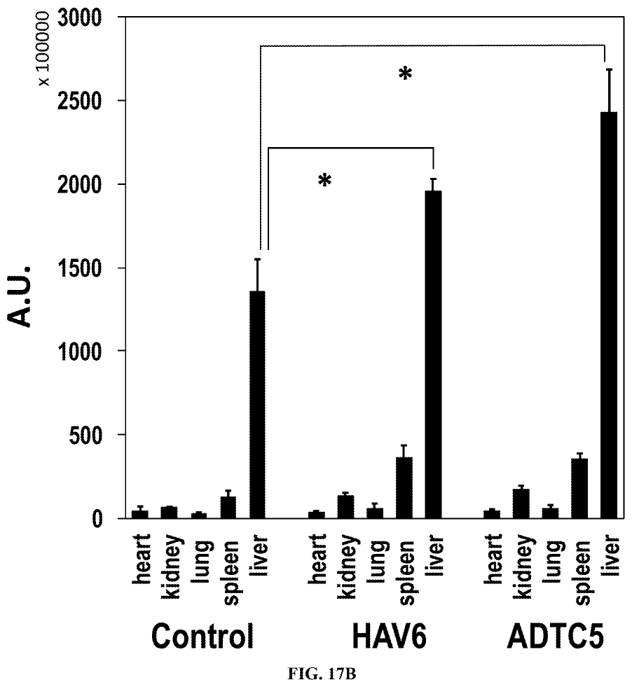

To evaluate molecules larger than lysozyme, 65 kDa albumin was delivered using HAV6 and ADTC5 in C57BL/6 mice compared to control (i.e., albumin alone) (FIG. 17A). The calibration curve was generated with 0.5 to 500 ng/mL labeled albumin in brain homogenates to generate a good linearity with R²>0.98. Prior to quantification of albumin deposition, the brains were subjected to perfusion process to remove the remaining albumin in the brain microvessels. The mice treated with albumin+ADTC5 showed a significantly higher albumin deposition (40.7±7.4 pmol/g brain) compared to albumin alone (11.8±1.0 pmol/g; p <0.05). Although it was not significant, the HAV6 group showed a trend of enhanced brain with brain deposition of 15.5±3.1 pmol/g compared to control (11.8±1.0 μmol/g brain (p=0.20)). These data also showed that ADTC5 was a better BBB modulator than HAV6 in delivering albumin.

The effects of HAV6 and ADTC5 peptides in the distribution of albumin in different organs were evaluated using NIRF quantitative imaging (FIG. 17B). The data indicated that HAV6 (p=0.04) and ADTC5 (p=0.04) significantly enhanced the distributions of albumin into the liver compared to control. There was no significant difference in albumin depositions between the liver ADTC5 group and the HAV6 group (p=0.15). Although the deposition in spleen is lower than in liver, the HAVS and ADTC5 groups both enhanced the deposition of albumin in the spleen compared to control.

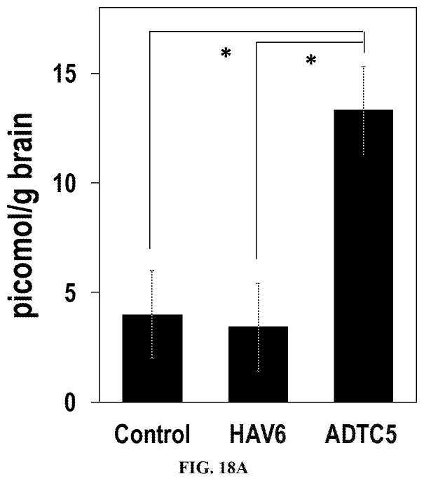

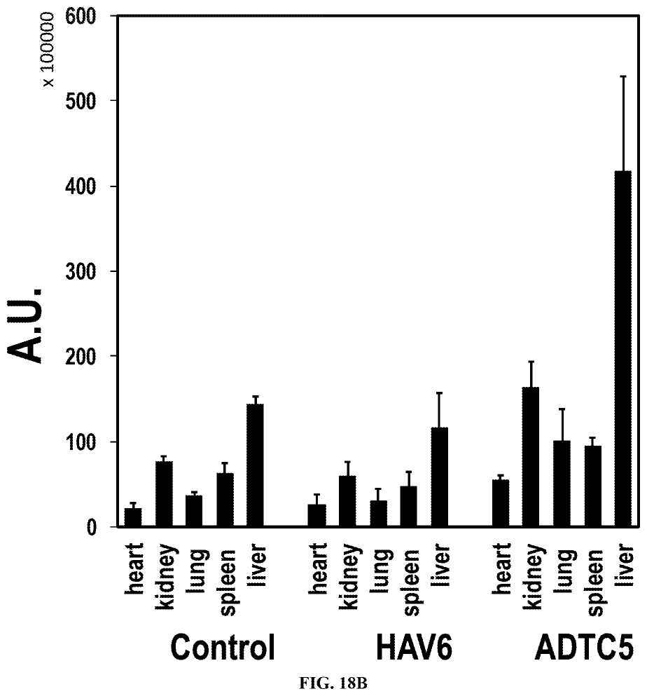

Brain Delivery of 150 kDa IgG mAb and Peripheral Organ Distributions. Because many mAbs have been utilized as therapeutics, there is high interest in improving their brain delivery. For quantitative determinations, a calibration curve for mAb was prepared with concentrations ranging from 10 to 200 ng/mL of IRDye800CW-IgG mAb spiked into blank brain homogenates. The calibration curve showed good linearity with R²≥0.99. The mice were perfused to remove any residual IgG mAb in the brain capillaries to avoid additional NIRF signal from protein in the capillaries. As in the previous study in SJL/elite, NIRF imaging signals from mAb in the brains of ADTC5+mAb-treated mice were higher than those of mAb-treated mice in C57BL/6 mice. The amounts of mAb in the brains of mice treated with ADTC5 +mAb (13.3±0.7 pmol/g) were significantly higher compared to those of HAV6+mAb (3.42±0.5 pmol/g; p<0.05) and mAb alone (4.0±0.4 pmol/g; p <0.05; FIG. 18A). HAV6 peptide was not able to deliver mAb (p >0.05) compared to control mAb (FIG. 18A). The enhancement of mAb brain deposition by ADTC5 is about three times that of control. ADTC5 showed a trend to enhance the distribution of mAb into liver compared to HAV6- (p=0.06) and control-treated animals (p=0.06) (FIG. 18B). The distributions of mAb in HAV6- and control-treated animals were about the same (p=0.54).

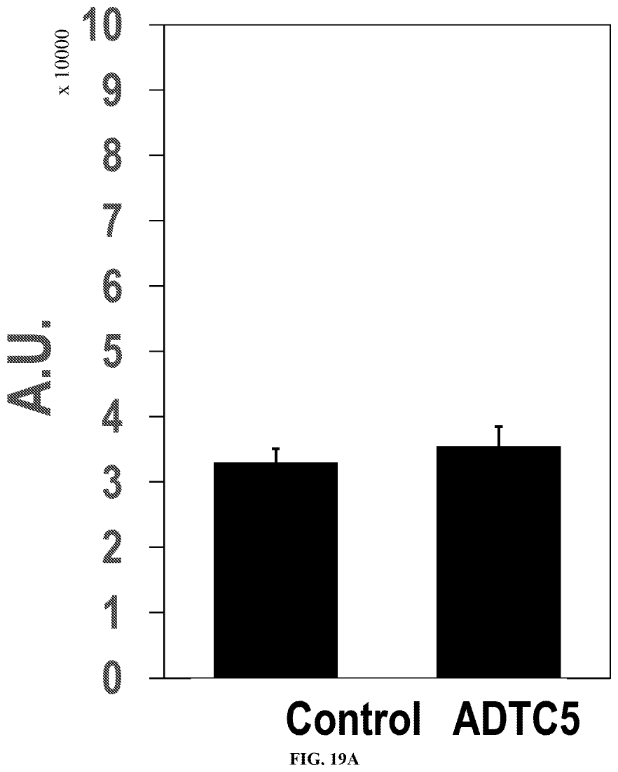

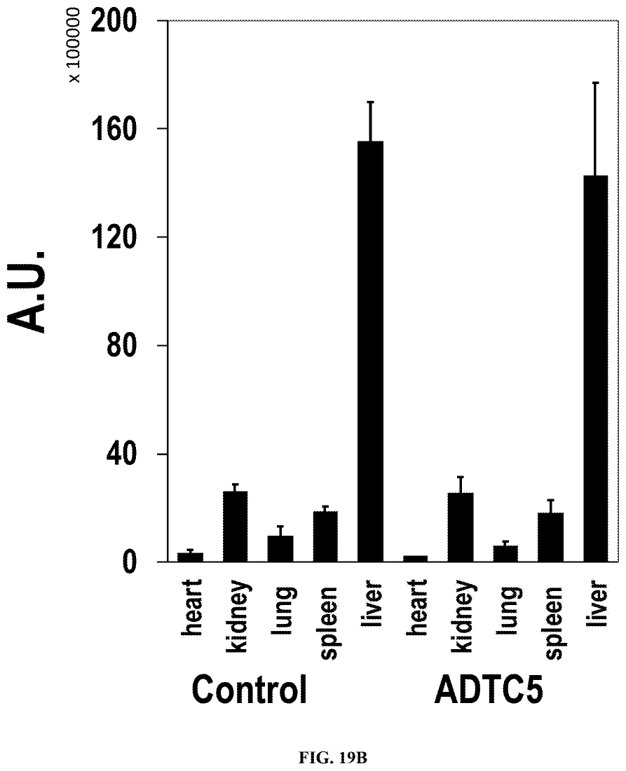

Brain Delivery of 220 kDa Fibronectin and Peripheral Organ Distributions. To find the larger limit of pore sizes made by ADTC5 peptide, the brain delivery of fibronectin (220 kDa) was evaluated in the presence and absence of ADTC5 (FIG. 19A). HAV6 was not investigated for delivering 220 kDa fibronectin because it cannot deliver 150 kDa mAb. ADTC5 did not enhance brain delivery of 220 kDa fibronectin because the NIRF signals for the ADTC5 +fibronectin group (35.498±3.001×10³ A.U.) was not different than that of fibronectin alone group (33.026±2.080×10³ A.U.) (FIG. 19A). The distributions of fibronectin were mostly in the liver, and ADTC5 did not influence the distribution of fibronectin in other organs (FIG. 19B).

It is expected that performing similar studies as described herein with HAVN1, HAVN2, ADTN1, ADTN2, and/or cyclic ADTHAV will provide results that are similar or significantly improved.

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:

A. A compound that is cyclo(1,6)SHAVSS (SEQ ID NO: 1; “HAVN1”) or a pharmaceutically acceptable salt thereof, cyclo(1,5)SHAVS (SEQ ID NO: 2; “HAVN2”) or a pharmaceutically acceptable salt thereof, cyclo(1,8)TPPVSHAV (SEQ ID NO: 3; “cyclic ADTHAV”) or a pharmaceutically acceptable salt thereof, cyclo(1,6)ADTPPV (SEQ ID NO: 4; “ADTN1”) or a pharmaceutically acceptable salt thereof, cyclo(1,5)DTPPV (SEQ ID NO: 5; “ADTN2”) or a pharmaceutically acceptable salt thereof, or acetyl-TPPVSHAV-NH₂ (SEQ ID NO: 6; “linear ADTHAV”) or a pharmaceutically acceptable salt thereof.
B. A composition comprising a compound of Paragraph A and a pharmaceutically acceptable carrier, optionally wherein the composition is formulated for one or more of parenteral administration, intravenous administration, subcutaneous administration, and oral administration, optionally wherein the composition is formulated in unit dosage form.
C. The composition of Paragraph B, wherein the composition further comprises one or more of a diagnostic agent and a therapeutic agent, optionally wherein a molar ratio of the compound to the diagnostic agent is about 5:1 to about 3,000:1, optionally wherein a molar ratio of the compound to the therapeutic agent is about 5:1 to about 3,000:1.
D. The composition of Paragraph B or Paragraph C, wherein the composition further comprises a small-molecule drug (i.e., a therapeutic compound less than 600 Daltons; e.g., adenanthin, daunomycin, doxorubicin, camptothecin, or a combination of any two or more thereof), a neuroregenerative molecule (e.g., brain-derived neurotrophic factor, nerve growth factor, insulin-like growth factor 1, or a combination of any two or more thereof), a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), a large protein (e.g., a lysozyme, a ApoE2 protein, albumin, an antibody (such as an antibody-drug conjugate), or a combination of any two or more thereof), or a combination of any two or more thereof, optionally wherein a molar ratio of the compound to the small-molecule drug is about 5:1 to about 3,000:1, optionally wherein a molar ratio of the compound to the neuroregenerative molecule is about 5:1 to about 3,000:1; optionally wherein a molar ratio of the compound to the medium-length peptide is about 5:1 to about 3,000:1; optionally wherein a molar ratio of the compound to the large protein is about 5:1 to about 3,000:1.
E. The composition of any one of Paragraphs B-D, wherein the composition further comprises one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503.
F. A pharmaceutical composition comprising an effective amount of a compound of Paragraph A and a pharmaceutically acceptable carrier, wherein the effective amount is effective for one or more of treating a brain disease, imaging a brain disease, and diagnosing a brain disease, optionally wherein the pharmaceutical composition is formulated in unit dosage form.
G. The pharmaceutical composition of Paragraph F, wherein the brain disease comprises one or more of a brain tumor (e.g., glioblastoma, medulloblastoma), Alzheimer's disease, multiple sclerosis, and Parkinson's disease.
H. The pharmaceutical composition of Paragraph F or Paragraph G, wherein the pharmaceutical composition further comprises one or more of a diagnostic agent and a therapeutic agent, optionally wherein a molar ratio of the compound to the diagnostic agent is about 5:1 to about 3,000:1, optionally wherein a molar ratio of the compound to the therapeutic agent is about 5:1 to about 3,000:1.
I. The pharmaceutical composition of any one of Paragraphs F-H, wherein the pharmaceutical composition further comprises one or more of an effective amount of a diagnostic agent and an effective amount of a therapeutic agent, wherein the effective amount is effective for one or more of treating a brain disease, imaging a brain disease, and diagnosing a brain disease.
J. The pharmaceutical composition of any one of Paragraphs F-I, wherein the pharmaceutical composition further comprises a small-molecule drug (i.e., a therapeutic compound less than 600 Daltons; e.g., adenanthin, daunomycin, doxorubicin, camptothecin, or a combination of any two or more thereof), a neuroregenerative molecule (e.g., brain-derived neurotrophic factor, nerve growth factor, insulin-like growth factor 1, or a combination of any two or more thereof), a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), a large protein (e.g., a lysozyme, a ApoE2 protein, albumin, an antibody (such as an antibody-drug conjugate), or a combination of any two or more thereof), or a combination of any two or more thereof, optionally wherein a molar ratio of the compound to the small-molecule drug is about 5:1 to about 3,000:1, optionally wherein a molar ratio of the compound to the neuroregenerative molecule is about 5:1 to about 3,000:1; optionally wherein a molar ratio of the compound to the medium-length peptide is about 5:1 to about 3,000:1; optionally wherein a molar ratio of the compound to the large protein is about 5:1 to about 3,000:1.
K. The pharmaceutical composition of any one of Paragraphs F-J, wherein the pharmaceutical composition further comprises an effective amount of a small-molecule drug (i.e., a therapeutic compound less than 600 Daltons; e.g., adenanthin, daunomycin, doxorubicin, camptothecin, or a combination of any two or more thereof), an effective amount of a neuroregenerative molecule (e.g., brain-derived neurotrophic factor, nerve growth factor, insulin-like growth factor 1, or a combination of any two or more thereof), an effective amount of a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), an effective amount of a large protein (e.g., a lysozyme, a ApoE2 protein, albumin, an antibody (such as an antibody-drug conjugate), or a combination of any two or more thereof), or a combination of any two or more thereof, wherein the effective amount is effective for one or more of treating a brain disease, imaging a brain disease, and diagnosing a brain disease.
L. The pharmaceutical composition of any one of Paragraphs F-K, wherein the pharmaceutical composition further comprises one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503.
M. The pharmaceutical composition of any one of Paragraphs F-L, wherein the pharmaceutical composition is formulated for one or more of parenteral administration, intravenous administration, subcutaneous administration, and oral administration.
N. The pharmaceutical composition of any one of Paragraphs F-M, wherein the pharmaceutical composition is formulated for intravenous administration and/or subcutaneous administration.
O. A method comprising administering a compound of Paragraph A to a subject suffering from a brain disease and/or administering a composition of any one of Paragraphs B-E to a subject suffering from a brain disease, optionally wherein about 0.01 mg/kg to about 100 mg/kg (mass of the compound/mass of the subject) of the compound is administered to the subject, optionally wherein about 0.01 mg/kg to about 20 mg/kg of the compound is administered to the subject.
P. The method of Paragraph 0, wherein the brain disease comprises one or more of a brain tumor (e.g., glioblastoma, medulloblastoma), Alzheimer's disease, multiple sclerosis, and Parkinson's disease.
Q. The method of Paragraph 0 or Paragraph P, wherein administering comprises one or more of parenteral administration, intravenous administration, subcutaneous administration, and oral administration.
R. The method of any one of Paragraphs O-Q, wherein the method comprises administering an effective amount of the compound to the subject and/or administering an effective amount of the composition to the subject, wherein the effective amount is effective for one or more of treating a brain disease, imaging a brain disease, and diagnosing a brain disease.
S. The method of any one of Paragraphs O-R, wherein the method further comprises administering one or more of an effective amount of a diagnostic agent and an effective amount of a therapeutic agent, wherein the effective amount is effective for one or more of treating a brain disease, imaging a brain disease, and diagnosing a brain disease, optionally wherein a molar ratio of the compound to the diagnostic agent is about 5:1 to about 3,000:1, optionally wherein a molar ratio of the compound to the therapeutic agent is about 5:1 to about 3,000:1.
T. The method of any one of Paragraphs O-S, wherein the method further comprises administering a small-molecule drug (i.e., a therapeutic compound less than 600 Daltons; e.g., adenanthin, daunomycin, doxorubicin, camptothecin, or a combination of any two or more thereof), a neuroregenerative molecule (e.g., brain-derived neurotrophic factor, nerve growth factor, insulin-like growth factor 1, or a combination of any two or more thereof), a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), a large protein (e.g., a lysozyme, a ApoE2 protein, albumin, an antibody (such as an antibody-drug conjugate), or a combination of any two or more thereof), or a combination of any two or more thereof, optionally wherein a molar ratio of the compound to the small-molecule drug is about 5:1 to about 3,000:1, optionally wherein a molar ratio of the compound to the neuroregenerative molecule is about 5:1 to about 3,000:1; optionally wherein a molar ratio of the compound to the medium-length peptide is about 5:1 to about 3,000:1; optionally wherein a molar ratio of the compound to the large protein is about 5:1 to about 3,000:1.
U. The method of any one of Paragraphs O-T, wherein the method further comprises administering an effective amount of a small-molecule drug (i.e., a therapeutic compound less than 600 Daltons; e.g., adenanthin, daunomycin, doxorubicin, camptothecin, or a combination of any two or more thereof), an effective amount of a neuroregenerative molecule (e.g., brain-derived neurotrophic factor, nerve growth factor, insulin-like growth factor 1, or a combination of any two or more thereof), an effective amount of a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), an effective amount of a large protein (e.g., a lysozyme, a ApoE2 protein, albumin, an antibody (such as an antibody-drug conjugate), or a combination of any two or more thereof), or a combination of any two or more thereof, wherein the effective amount is effective for one or more of treating a brain disease, imaging a brain disease, and diagnosing a brain disease.
V. The method of any one of Paragraphs O-U, wherein the method further comprises administering one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503.
W. The method of any one of Paragraphs O-V, wherein the method further comprises administering an effective amount of one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503, wherein the effective amount is effective for one or more of treating a brain disease, imaging a brain disease, and diagnosing a brain disease.
X. The method of any one of Paragraphs O-W, wherein administering the compound does not comprise intracerebroventricular injection.
Y. The method of any one of Paragraphs O-X, wherein administering the composition does not comprise intracerebroventricular injection.
Z. The method of any one of Paragraphs O-Y, wherein the method does not comprise intracerebroventricular injection.
AA. A method comprising administering a pharmaceutical composition of any one of Pargraphs F-N to a subject suffering from a brain disease, optionally wherein about 0.01 mg/kg to about 100 mg/kg (mass of the compound/mass of the subject) of the compound is administered to the subject, optionally wherein about 0.01 mg/kg to about 20 mg/kg (mass of the compound/mass of the subject) of the compound is administered to the subject.
AB. The method of Paragraph AA, wherein the brain disease comprises one or more of a brain tumor (e.g., glioblastoma, medulloblastoma), Alzheimer's disease, multiple sclerosis, and Parkinson's disease.
AC. The method of Paragraph AA or Paragraph AB, wherein administering comprises parenteral administration, intravenous administration, subcutaneous administration, or oral administration.
AD. The method of any one of Paragraphs AA-AC, wherein the method further comprises administering one or more of an effective amount of a diagnostic agent and an effective amount of a therapeutic agent, wherein the effective amount is effective for one or more of treating a brain disease, imaging a brain disease, and diagnosing a brain disease, optionally wherein a molar ratio of the compound to the diagnostic agent is about 5:1 to about 3,000:1, optionally wherein a molar ratio of the compound to the therapeutic agent is about 5:1 to about 3,000:1.
AE. The method of any one of Paragraphs AA-AD, wherein the method further comprises administering a small-molecule drug (i.e., a therapeutic compound less than 600 Daltons; e.g., adenanthin, daunomycin, doxorubicin, camptothecin, or a combination of any two or more thereof), a neuroregenerative molecule (e.g., brain-derived neurotrophic factor, nerve growth factor, insulin-like growth factor 1, or a combination of any two or more thereof), a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), a large protein (e.g., a lysozyme, a ApoE2 protein, albumin, an antibody (such as an antibody-drug conjugate), or a combination of any two or more thereof), or a combination of any two or more thereof, optionally wherein a molar ratio of the compound to the small-molecule drug is about 5:1 to about 3,000:1, optionally wherein a molar ratio of the compound to the neuroregenerative molecule is about 5:1 to about 3,000:1; optionally wherein a molar ratio of the compound to the medium-length peptide is about 5:1 to about 3,000:1; optionally wherein a molar ratio of the compound to the large protein is about 5:1 to about 3,000:1.
AF. The method of any one of Paragraphs AA-AE, wherein the method further comprises administering an effective amount of a small-molecule drug (i.e., a therapeutic compound less than 600 Daltons; e.g., adenanthin, daunomycin, doxorubicin, camptothecin, or a combination of any two or more thereof), an effective amount of a neuroregenerative molecule (e.g., brain-derived neurotrophic factor, nerve growth factor, insulin-like growth factor 1, or a combination of any two or more thereof), an effective amount of a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), an effective amount of a large protein (e.g., a lysozyme, a ApoE2 protein, albumin, an antibody (such as an antibody-drug conjugate), or a combination of any two or more thereof), or a combination of any two or more thereof, wherein the effective amount is effective for one or more of treating a brain disease, imaging a brain disease, and diagnosing a brain disease.
AG. The method of any one of Paragraphs AA-AF, wherein the method further comprises administering one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503.
AH. The method of any one of Paragraphs AA-AG, wherein the method further comprises administering an effective amount of one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503, wherein the effective amount is effective for one or more of treating a brain disease, imaging a brain disease, and diagnosing a brain disease.
AI. The method of any one of Paragraphs AA-AH, wherein administering the pharmaceutical composition does not comprise intracerebroventricular injection.
AJ. The method of any one of Paragraphs AA-AI, wherein the method does not comprise intracerebroventricular injection.
AK. A pharmaceutical composition comprising a pharmaceutically acceptable carrier and an effective amount of one or more of acetyl-SHAVSS-NH₂ (SEQ ID NO: 7; “HAVE”) or a pharmaceutically acceptable salt thereof, cyclo(1,7)acetyl-CDTPPVC-NH₂ (SEQ ID NO: 8; “ADTC5”) or a pharmaceutically acceptable salt thereof, acetyl-SHAVAS-NH₂ (SEQ ID NO: 9; “HAV4”) or a pharmaceutically acceptable salt thereof, and cyclo(1,6)acetyl-CSHAVC-NH₂ (SEQ ID NO: 10; “cHAVc3”) or a pharmaceutically acceptable salt thereof (referred to collectively hereafter in dependent Paragraphs as “the compound(s)”), wherein the effective amount is effective for one or more of treating a brain disease, imaging a brain disease, and diagnosing a brain disease, optionally wherein the pharmaceutical composition is formulated in unit dosage form.
AL. The pharmaceutical composition of Paragraph AK, wherein the brain disease comprises one or more of a brain tumor (e.g., glioblastoma, medulloblastoma), Alzheimer's disease, multiple sclerosis, and Parkinson's disease.
AM. The pharmaceutical composition of Paragraph AK or Paragraph AL, wherein the composition further comprises one or more of a diagnostic agent and a therapeutic agent, optionally wherein a molar ratio of the compound(s) to the diagnostic agent is about 5:1 to about 3,000:1, optionally wherein a molar ratio of the compound(s) to the therapeutic agent is about 5:1 to about 3,000:1.
AN. The pharmaceutical composition of any one of Paragraphs AK-AM, wherein the pharmaceutical composition further comprises one or more of an effective amount of a diagnostic agent and an effective amount of a therapeutic agent, wherein the effective amount is effective for one or more of treating a brain disease, imaging a brain disease, and diagnosing a brain disease, optionally wherein a molar ratio of the compound(s) to the diagnostic agent is about 5:1 to about 3,000:1, optionally wherein a molar ratio of the compound(s) to the therapeutic agent is about 5:1 to about 3,000:1.
AO. The pharmaceutical composition of any one of Paragraphs AK-AN, wherein the pharmaceutical composition further comprises a small-molecule drug (i.e., a therapeutic compound less than 600 Daltons; e.g., adenanthin, daunomycin, doxorubicin, camptothecin, or a combination of any two or more thereof), a neuroregenerative molecule (e.g., brain-derived neurotrophic factor, nerve growth factor, insulin-like growth factor 1, or a combination of any two or more thereof), a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), a large protein (e.g., a lysozyme, a ApoE2 protein, albumin, an antibody (such as an antibody-drug conjugate), or a combination of any two or more thereof), or a combination of any two or more thereof, optionally wherein a molar ratio of the compound(s) to the small-molecule drug is about 5:1 to about 3,000:1, optionally wherein a molar ratio of the compound(s) to the neuroregenerative molecule is about 5:1 to about 3,000:1; optionally wherein a molar ratio of the compound(s) to the medium-length peptide is about 5:1 to about 3,000:1; optionally wherein a molar ratio of the compound(s) to the large protein is about 5:1 to about 3,000:1.
AP. The pharmaceutical composition of any one of Paragraphs AK-AO, wherein the pharmaceutical composition further comprises an effective amount of a small-molecule drug (i.e., a therapeutic compound less than 600 Daltons; e.g., adenanthin, daunomycin, doxorubicin, camptothecin, or a combination of any two or more thereof), an effective amount of a neuroregenerative molecule (e.g., brain-derived neurotrophic factor, nerve growth factor, insulin-like growth factor 1, or a combination of any two or more thereof), an effective amount of a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), an effective amount of a large protein (e.g., a lysozyme, a ApoE2 protein, albumin, an antibody (such as an antibody-drug conjugate), or a combination of any two or more thereof), or a combination of any two or more thereof, wherein the effective amount is effective for one or more of treating a brain disease, imaging a brain disease, and diagnosing a brain disease.
AQ. The pharmaceutical composition of any one of Paragraphs AK-AP, wherein the pharmaceutical composition further comprises one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503; optionally wherein the pharmaceutical composition further comprises an effective amount one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503, wherein the effective amount is effective for one or more of treating a brain disease, imaging a brain disease, and diagnosing a brain disease.
AR. The pharmaceutical composition of any one of Paragraphs AK-AQ, wherein the pharmaceutical composition is formulated for one or more of parenteral administration, intravenous administration, subcutaneous administration, and oral administration.
AS. The pharmaceutical composition of any one of Paragraphs AK-AR, wherein the pharmaceutical composition is formulated for intravenous administration and/or subcutaneous administration.
AT. A method comprising administering to a subject suffering from a brain disease one or more of acetyl-SHAVSS-NH₂ (SEQ ID NO: 7; “HAV6”) or a pharmaceutically acceptable salt thereof, cyclo(1,7)acetyl-CDTPPVC-NH₂ (SEQ ID NO: 8; “ADTC5”) or a pharmaceutically acceptable salt thereof, acetyl-SHAVAS-NH₂ (SEQ ID NO: 9; “HAV4”) or a pharmaceutically acceptable salt thereof, and cyclo(1,6)acetyl-CSHAVC-NH₂ (SEQ ID NO: 10; “cHAVc3”) or a pharmaceutically acceptable salt thereof (referred to collectively hereafter in dependent Paragraphs as “the compound(s)”), optionally wherein about 0.01 mg/kg to about 100 mg/kg ([mass of the one or more HAV6 or a pharmaceutically acceptable salt thereof, ADTC5 or a pharmaceutically acceptable salt thereof, HAV4 or a pharmaceutically acceptable salt thereof, and cHAVc3 or a pharmaceutically acceptable salt thereof]/[mass of the subject]) is administered to the subject.
AU. The method of Paragraph AT, wherein the brain disease comprises one or more of a brain tumor (e.g., glioblastoma, medulloblastoma), Alzheimer's disease, multiple sclerosis, and Parkinson's disease.
AV. The method of Paragraph AT or Paragraph AU, wherein administering comprises one or more of parenteral administration, intravenous administration, subcutaneous administration, and oral administration.
AW. The method of any one of Paragraphs AT-AV, wherein the method comprises administering an effective amount of the compound(s) to the subject and/or administering an effective amount of the composition to the subject, wherein the effective amount is effective for one or more of treating a brain disease, imaging a brain disease, and diagnosing a brain disease.
AX. The method of any one of Paragraphs AT-AW, wherein the method further comprises administering one or more of an effective amount of a diagnostic agent and an effective amount of a therapeutic agent, wherein the effective amount is effective for one or more of treating a brain disease, imaging a brain disease, and diagnosing a brain disease, optionally wherein a molar ratio of the compound(s) to the diagnostic agent is about 5:1 to about 3,000:1, optionally wherein a molar ratio of the compound(s) to the therapeutic agent is about 5:1 to about 3,000:1.
AY. The method of any one of Paragraphs AT-AX, wherein the method further comprises administering a small-molecule drug (i.e., a therapeutic compound less than 600 Daltons; e.g., adenanthin, daunomycin, doxorubicin, camptothecin, or a combination of any two or more thereof), a neuroregenerative molecule (e.g., brain-derived neurotrophic factor, nerve growth factor, insulin-like growth factor 1, or a combination of any two or more thereof), a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), a large protein (e.g., a lysozyme, a ApoE2 protein, albumin, an antibody (such as an antibody-drug conjugate), or a combination of any two or more thereof), or a combination of any two or more thereof, optionally wherein a molar ratio of the compound(s) to the small-molecule drug is about 5:1 to about 3,000:1, optionally wherein a molar ratio of the compound(s) to the neuroregenerative molecule is about 5:1 to about 3,000:1; optionally wherein a molar ratio of the compound(s) to the medium-length peptide is about 5:1 to about 3,000:1; optionally wherein a molar ratio of the compound(s) to the large protein is about 5:1 to about 3,000:1.
AZ. The method of any one of Paragraphs AT-AY, wherein the method further comprises administering an effective amount of a small-molecule drug (i.e., a therapeutic compound less than 600 Daltons; e.g., adenanthin, daunomycin, doxorubicin, camptothecin, or a combination of any two or more thereof), an effective amount of a neuroregenerative molecule (e.g., brain-derived neurotrophic factor, nerve growth factor, insulin-like growth factor 1, or a combination of any two or more thereof), an effective amount of a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), an effective amount of a large protein (e.g., a lysozyme, a ApoE2 protein, albumin, an antibody (such as an antibody-drug conjugate), or a combination of any two or more thereof), or a combination of any two or more thereof, wherein the effective amount is effective for one or more of treating a brain disease, imaging a brain disease, and diagnosing a brain disease.
BA. The method of any one of Paragraphs AT-AZ, wherein the method further comprises administering one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503.
BB. The method of any one of Paragraphs AT-BA, wherein the method further comprises administering an effective amount of one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503, wherein the effective amount is effective for one or more of treating a brain disease, imaging a brain disease, and diagnosing a brain disease.
BC. The method of any one of Paragraphs AT-BB, wherein administering the compound(s) does not comprise intracerebroventricular injection.
BD. The method of any one of Paragraphs AT-BC, wherein the method does not comprise intracerebroventricular injection.
BE. A method comprising administering a pharmaceutical composition of any one of Paragraphs AK-AS to a subject suffering from a brain disease, optionally wherein about 0.01 mg/kg to about 100 mg/kg ([mass of the one or more HAVE or a pharmaceutically acceptable salt thereof, ADTC5 or a pharmaceutically acceptable salt thereof, HAV4 or a pharmaceutically acceptable salt thereof, and cHAVc3 or a pharmaceutically acceptable salt thereof ]/[mass of the subject]) is administered to the subject
BF. The method of Paragraph BE, wherein the brain disease comprises one or more of a brain tumor (e.g., glioblastoma, medulloblastoma), Alzheimer's disease, multiple sclerosis, and Parkinson's disease.
BG. The method of Paragraph BE or Paragraph BF, wherein administering comprises parenteral administration, intravenous administration, subcutaneous administration, or oral administration.
BH. The method of any one of Paragraphs BE-BG, wherein the method further comprises administering one or more of an effective amount of a diagnostic agent and an effective amount of a therapeutic agent, wherein the effective amount is effective for one or more of treating a brain disease, imaging a brain disease, and diagnosing a brain disease, optionally wherein a molar ratio of the compound(s) to the diagnostic agent is about 5:1 to about 3,000:1, optionally wherein a molar ratio of the compound(s) to the therapeutic agent is about 5:1 to about 3,000:1.
BI. The method of any one of Paragraphs BE-BH, wherein the method further comprises administering a small-molecule drug (i.e., a therapeutic compound less than 600 Daltons; e.g., adenanthin, daunomycin, doxorubicin, camptothecin, or a combination of any two or more thereof), a neuroregenerative molecule (e.g., brain-derived neurotrophic factor, nerve growth factor, insulin-like growth factor 1, or a combination of any two or more thereof), a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), a large protein (e.g., a lysozyme, a ApoE2 protein, albumin, an antibody (such as an antibody-drug conjugate), or a combination of any two or more thereof), or a combination of any two or more thereof, optionally wherein a molar ratio of the compound(s) to the small-molecule drug is about 5:1 to about 3,000:1, optionally wherein a molar ratio of the compound(s) to the neuroregenerative molecule is about 5:1 to about 3,000:1; optionally wherein a molar ratio of the compound(s) to the medium-length peptide is about 5:1 to about 3,000:1; optionally wherein a molar ratio of the compound(s) to the large protein is about 5:1 to about 3,000:1.
BJ. The method of any one of Paragraphs BE-BI, wherein the method further comprises administering an effective amount of a small-molecule drug (i.e., a therapeutic compound less than 600 Daltons; e.g., adenanthin, daunomycin, doxorubicin, camptothecin, or a combination of any two or more thereof), an effective amount of a neuroregenerative molecule (e.g., brain-derived neurotrophic factor, nerve growth factor, insulin-like growth factor 1, or a combination of any two or more thereof), an effective amount of a medium-length peptide (i.e., a peptide of about 7 to about 12 amino acids; e.g., oxytocin, exenatide, liraglutide, octreotide, leprolide, calcitonin, vasopressin, enfuvirtide, integrilin, goserelin, gonadotropin-releasing hormone, enkephalin, bivalirudin, carbetocin, desmopressin, teriparatide, semorelin, nesiritide, pramlintide, gramacidin D, icatibant, cetrorelix, tetracosactide, or a combination of any two or more thereof), an effective amount of a large protein (e.g., a lysozyme, a ApoE2 protein, albumin, an antibody (such as an antibody-drug conjugate), or a combination of any two or more thereof), or a combination of any two or more thereof, wherein the effective amount is effective for one or more of treating a brain disease, imaging a brain disease, and diagnosing a brain disease.
BK. The method of any one of Paragraphs BE-BJ, wherein the method further comprises administering one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503.
BL. The method of any one of Paragraphs BE-BK, wherein the method further comprises administering an effective amount of one or more of belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, and VX15/2503, wherein the effective amount is effective for one or more of treating a brain disease, imaging a brain disease, and diagnosing a brain disease.
BM. The method of any one of Paragraphs BE-BL, wherein administering the pharmaceutical composition does not comprise intracerebroventricular injection.
BN. The method of any one of Paragraphs BE-BM, wherein the method does not comprise intracerebroventricular injection.

Other embodiments are set forth in the following claims, along with the full scope of equivalents to which such claims are entitled.

1. A compound that is cyclo(1,6)SHAVSS (SEQ ID NO: 1; “HAVN1”) or a pharmaceutically acceptable salt thereof, cyclo(1,5)SHAVS (SEQ ID NO: 2; “HAVN2”) or a pharmaceutically acceptable salt thereof, cyclo(1,8)TPPVSHAV (SEQ ID NO: 3;
“cyclic ADTHAV”) or a pharmaceutically acceptable salt thereof, cyclo(1,6)ADTPPV (SEQ ID NO: 4; “ADTN1”) or a pharmaceutically acceptable salt thereof, cyclo(1,5)DTPPV (SEQ ID NO: 5; “ADTN2”) or a pharmaceutically acceptable salt thereof, or acetyl-TPPVSHAV-NH₂ (SEQ ID NO: 6; “linear ADTHAV”) or a pharmaceutically acceptable salt thereof.

2. A composition comprising a compound of claim 1 and a pharmaceutically acceptable carrier.

3. The composition of claim 2, wherein the composition further comprises one or more of a diagnostic agent and a therapeutic agent.

4. The composition of claim 2, wherein the composition further comprises a small-molecule drug, adenanthin, daunomycin, doxorubicin, camptothecin, a neuroregenerative molecule, brain-derived neurotrophic factor, nerve growth factor, insulin-like growth factor 1, an antibody, or a combination of any two or more thereof.

5. The composition of claim 2, wherein the composition further comprises belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, or VX15/2503.

6. A pharmaceutical composition comprising an effective amount of a compound of claim 1 and a pharmaceutically acceptable carrier, wherein the effective amount is effective for one or more of treating a brain disease, imaging a brain disease, and diagnosing a brain disease.

7. The pharmaceutical composition of claim 6, wherein the brain disease comprises one or more of a glioblastoma, a medulloblastoma, Alzheimer's disease, multiple sclerosis, and Parkinson's disease.

8. The pharmaceutical composition of claim 6, wherein the pharmaceutical composition further comprises one or more of an effective amount of a diagnostic agent and an effective amount of a therapeutic agent, wherein the effective amount is effective for one or more of treating a brain disease, imaging a brain disease, and diagnosing a brain disease.

9. The pharmaceutical composition of claim 8, wherein the therapeutic agent comprises adenanthin, daunomycin, doxorubicin, camptothecin, brain-derived neurotrophic factor, nerve growth factor, insulin-like growth factor 1, or a combination of any two or more thereof.

10. The pharmaceutical composition of claim 8, wherein the therapeutic agent comprises belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, VX15/2503, or a combination of any two or more thereof.

11. The pharmaceutical composition of claim 6, wherein the pharmaceutical composition is formulated for one or more of parenteral administration, intravenous administration, subcutaneous administration, and oral administration.

12. The pharmaceutical composition of claim 8, wherein the pharmaceutical composition is formulated for intravenous administration.

13.-14. (canceled)

15. A method comprising administering an effective amount of compound of claim 1 to a subject suffering from a brain disease, wherein the effective amount is effective for one or more of treating a brain disease, imaging a brain disease, and diagnosing a brain disease.

16. The method of claim 15, wherein the brain disease comprises one or more of a brain tumor, Alzheimer's disease, multiple sclerosis, and Parkinson's disease.

17. The method of claim 15, wherein the method further comprises administering one or more of an effective amount of a diagnostic agent and an effective amount of a therapeutic agent, wherein the effective amount is effective for one or more of treating a brain disease, imaging a brain disease, and diagnosing a brain disease.

18. The method of claim 17, wherein the diagnostic agent and/or therapeutic agent comprises a small-molecule drug, a neuroregenerative molecule, an antibody, or a combination of any two or more thereof.

19. The method of claim 17, wherein the method further comprises administering the therapeutic agent, wherein the therapeutic agent comprises belimumab, mogamulizumab, blinatumomab, ibritumomab tiuxetan, obinutuzumab, ofatumumab, rituximab, inotuzumab ozogamicin, moxetumomab pasudotox, brentuximab vedotin, daratumumab, ipilimumab, cetuximab, necitumumab, panitumumab, dinutuximab, pertuzumab, trastuzumab, trastuzumab emtansine, siltuximab, cemiplimab, nivolumab, pembrolizumab, olaratumab, atezolizumab, avelumab, durvalumab, capromab pendetide, elotuzumab, denosumab, ziv-aflibercept, bevacizumab, ramucirumab, tositumomab, gemtuzumab ozogamicin, alemtuzumab, cixutumumab, girentuximab, nimotuzumab, catumaxomab, etaracizumab, crenezumab, bapineuzumab, solanezumab, gantenerumab, ponezumab, BAN2401, aducanumab, ranibizumab, anti-Nogo-A, anti-LINGO-1, sHIgM22, VX15/2503, or a combination of any two or more thereof.

20. The method of claim 15, wherein administering the compound does not comprise intracerebroventricular injection.

21. The method of claim 15, wherein the method does not comprise intracerebroventricular injection.

22.-34. (canceled)

35. A pharmaceutical composition comprising a pharmaceutically acceptable carrier and an effective amount of one or more of acetyl-SHAVSS-NH₂ (SEQ ID NO: 7;
“HAVE”) or a pharmaceutically acceptable salt thereof, cyclo(1,7)acetyl-CDTPPVC-NH₂ (SEQ ID NO: 8; “ADTC5”) or a pharmaceutically acceptable salt thereof, acetyl-SHAVAS-NH₂ (SEQ ID NO: 9; “HAV4”) or a pharmaceutically acceptable salt thereof, and cyclo(1,6)acetyl-CSHAVC-NH₂ (SEQ ID NO: 10; “cHAVc3”) or a pharmaceutically acceptable salt thereof, wherein the effective amount is effective for treating Alzheimer's disease, multiple sclerosis, and/or Parkinson's disease.

36.-42. (canceled)


SEQUENCE LISTING (10 sequences)

SEQ ID NO: 1 - protein, 6 aa, Artificial Sequence
  Description of Artificial Sequence Synthetic peptide
SHAVSS

SEQ ID NO: 2 - protein, 5 aa, Artificial Sequence
  Description of Artificial Sequence Synthetic peptide
SHAVS

SEQ ID NO: 3 - protein, 8 aa, Artificial Sequence
  Description of Artificial Sequence Synthetic peptide
TPPVSHAV

SEQ ID NO: 4 - protein, 6 aa, Artificial Sequence
  Description of Artificial Sequence Synthetic peptide
ADTPPV

SEQ ID NO: 5 - protein, 5 aa, Artificial Sequence
  Description of Artificial Sequence Synthetic peptide
DTPPV

SEQ ID NO: 6 - protein, 8 aa, Artificial Sequence
  Description of Artificial Sequence Synthetic peptide
TPPVSHAV

SEQ ID NO: 7 - protein, 6 aa, Artificial Sequence
  Description of Artificial Sequence Synthetic peptide
SHAVSS

SEQ ID NO: 8 - protein, 7 aa, Artificial Sequence
  Description of Artificial Sequence Synthetic peptide
CDTPPVC

SEQ ID NO: 9 - protein, 6 aa, Artificial Sequence
  Description of Artificial Sequence Synthetic peptide
SHAVAS

SEQ ID NO: 10 - protein, 6 aa, Artificial Sequence
  Description of Artificial Sequence Synthetic peptide
CSHAVC